(19) United States
(12) Patent Application Publication
(10) Pub. No.: US 2026/0286288 A1
(43) Pub. Date: Sep. 24, 2026

(54) REGULATED CELLS EXPRESSING CYTOKINES

(71) Applicant: William Marsh Rice University, Houston, TX (US)

(72) Inventors: Omid VEISEH, Houston, TX (US); Amanda NASH, Houston, TX (US); Fangheng HU, Houston, TX (US)

(21) Appl. No.: 19/473,975

(22) PCT Filed: Apr. 12, 2024
(86) PCT No.: PCT/US2024/024216

§ 371 Date: Oct. 9, 2025

Related U.S. Application Data
(60) Provisional application No. 63/495,924, filed on Apr. 13, 2023.

(51) Int. Cl. C12N 5/00; A61K 35/30; A61P 35/00; C07K 14/54; C07K 14/55; C07K 16/28; C12N 9/64

(52) U.S. Cl. CPC C12N 5/0012; A61K 35/30; A61P 35/00; C07K 14/5434; C07K 14/55; C07K 16/2818; C12N 9/6472; C12Y 304/22062

(57) ABSTRACT

The present disclosure relates to compositions of encapsulated cells designed to deliver proteins of interest under the control of an inducible kill switch.

### PRIORITY CLAIM

This application claims benefit of priority to U.S. Provisional Application Ser. No. 63/495,924, filed Apr. 13, 2023, the entire contents of which are hereby incorporated by reference.

### GOVERNMENT SUPPORT CLAUSE

This invention was made with government support under Grant No. AY1AX000003 awarded by the National Institutes of Health-Advanced Research Projects Agency for Health. The government has certain rights in the invention.

### SEQUENCE LISTING

This application contains a Sequence Listing XML, which has been submitted electronically and is hereby incorporated by reference in its entirety. Said XML Sequence Listing, created on Apr. 11, 2024, is named RICEP0131WO.xml and is 89,106 bytes in size.

### BACKGROUND

### I. Field

The present disclosure relates to the fields of biology, medicine, bioengineering and medicals devices. More particular, it relates to the development and use of compositions designed to deliver antigenic therapeutic reagents to a subject and protect them from immune responses generated by the host. In particular, the constructs are designed to degrade over time or upon a particular signal, thereby providing control of the length of time the therapeutic agent is delivered to the subject.

### II. Related Art

Advances in biomedical research have led to methods for localized and targeted therapies for the treatment of diseases, such as cancer. However, in many instances, the percentage of patients that are responsive to these approaches remains modest (Park et al., Sci. Transl. Med. 10 (433) 2018).

One approach involves the use of implantable devices to deliver therapeutic agents, however, a fundamental barrier to successful device-based therapies is the inability to deliver a sustained amount of therapeutics that do not have a systemic toxic impact on the subject. Thus, there is a need for identifying new compositions and methods to enhance the delivery, distribution, and/or efficacy of therapeutic agents.

### SUMMARY OF THE INVENTION

In some embodiments, the disclosure provides for a composition comprising a population of encapsulated cells comprising a heterologous nucleic acid molecule encoding a protein of interest and an inducible kill switch.

In some embodiments, the disclosure provides for a composition comprising a population of encapsulated cells comprising a plurality of heterologous nucleic acid molecules encoding an IL-2 and an inducible kill switch. In some embodiments, the disclosure provides for a composition comprising a population of encapsulated cells comprising a plurality of heterologous nucleic acid molecules encoding an IL-12 and an inducible kill switch.

In some embodiments, the disclosure provides for a composition comprising two or more populations of encapsulated cells wherein a first population of encapsulated cells comprises an inducible kill switch and a first heterologous nucleic acid molecule encoding a first cytokine; and a second population of encapsulated cells comprises an inducible kill switch and a second heterologous nucleic acid molecule encoding a second cytokine, or an antibody that binds to a checkpoint inhibitor.

In some embodiments, the disclosure provides for a method of treating a subject with a disease, comprising administering to the subject any of the populations of encapsulated cells, or compositions disclosed herein.

In some embodiments, the disclosure provides for a method of treating a subject with a disease by generating memory immunity, the method comprising implanting any of the populations of encapsulated cells, or compositions disclosed herein.

In some embodiments, the disclosure provides a method of providing systemic treatment to a subject with cancer, the method comprising implanting in the intraperitoneal space of the subject any of the populations of encapsulated cells, or compositions disclosed herein, whereby the any of the populations of encapsulated cells, or compositions disclosed herein stimulate the activation of immune cells in the intraperitoneal space and the immune cells migrate to a region of the subject that is distal to the intraperitoneal space to treat the cancer systematically in the subject.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a series of two annotated photographs of two populations of cytokine-producing, kill switch-inducible cells, the first population (left) not having been exposed to small molecule kill switch inducer AP1903, and the second population (right) less than 24 hours after exposure to 100 nM AP1903.

FIG. 2 is a graph of mIL-2 concentration (pg/mL) of the two cell populations of FIG. 1, the first population (right) not having been exposed to AP1903 and the second population (left) less than 24 hours after exposure to 100 nM AP1903. The second population, exposed to AP1903, exhibits decreased mIL-2 production as compared to the first population.

FIG. 3A and FIG. 3B are graphs of mIL-2 concentration (pg/mL) over time of four alginate-encapsulated, cytokine-producing, kill switch-inducible cell populations exposed to 0 nM, 10 nM, 100 nM, and 1000 nM of small molecule kill switch inducer AP1903. All three cell populations exposed to AP1903 exhibit decreased mIL-2 production as compared to the control population.

FIG. 4 is a graph of weight (mg) of intraperitoneal metastatic tumors in mice after one week of: no treatment; treatment with a population of alginate-encapsulated cells producing mIL-12 (RPE-mIL12); treatment with a population of alginate-encapsulated cells producing mIL-2 (RPE-mIL2); and treatment with a population of alginate-encapsulated cells producing mIFNg (RPE-mIFNg). Tumors were extracted on Day 7 post-treatment and weighed using an analytical balance. Treatment groups demonstrated reduced tumor weight as compared to untreated group.

FIG. 5 is a graph of weight (mg) of intraperitoneal metastatic tumors in mice after one week of: sham treatment; treatment with alginate-encapsulated cells producing mIL-15 (RPE-mIL15); treatment with base cell lines (RPE); and treatment with alginate-encapsulated cells producing mTNF-α (RPE-mTNFa). Tumors were extracted on Day 7 post-treatment and weighed using an analytical balance. Treatment groups demonstrated no significant reduction in tumor weight as compared to sham treatment group.

FIG. 6 shows viability of encapsulated cells following administration of 0.4 mg/ml AP1903, or lack thereof.

FIG. 7A and FIG. 7B are graphs of IL-2 concentration (pg/mL) over time of four alginate-encapsulated, cytokine-producing, kill switch-inducible cell populations exposed to 0 nM, 10 nM, 100 nM, and 1000 nM of small molecule kill switch inducer AP1903.

FIG. 8A and FIG. 8B show validation of mouse IL-12 production of RPE-mIL12-ks cells. ELISAs were performed to measure the production rate of ARPE-19 cells transfected by IL-12 expression plasmids with caspase-9 suicide gene. Data is shown in pg/cell/day. FIG. 1A is a schematic of IL-12 kill switch plasmids. CAG: CMV enhancer, T2A: self-cleaving 2A peptide, iCasp9: inducible caspase-9 suicide gene. FIG. 1B shows ARPE-19 cells transfected with IL-12 kill switch vector. Mouse IL-12 value in media were assayed with ELISA 24 hours later (n=3).

FIGS. 9A-C show that AP1903 small molecule is highly effective at inducing cell death in RPE-mIL 12-ks cells. FIG. 9A is an in vitro assay. Images of RPE-mIL 12-ks cells via the microscope 24 hours after 0 nM (left) or 10 nM AP1903 (right) administration. FIG. 9B is an in vitro assay. Cell viability measured after lysing encapsulated RPE-IL12-ks via cell counter 24 hours post administration (n=3). FIG. 9C is an in vivo assay. Encapsulated IL12-ks cells respond to 0.4 mg/mL AP1903 in less than 24 hours in healthy mice (n=3).

FIG. 10A and FIG. 10B show development of encapsulated RPE-mIL 12-ks cells with tunable local production. FIG. 10A shows capsules containing RPE cells. Bright and Dark field images of RPE-mIL12-ks cells encapsulated at 42e6 cells/mL of SLG20 alginate. Images acquired at 2× magnification; scale bar is 2000 μm. FIG. 10B shows in vivo mIL-12 concentrations as a function of capsule dose (n=5) 24 hours after implantation.

FIGS. 11A-C show validation of cellular anti-PD1 antibody production. ELISAs were performed to measure the production rate of ARPE-19 cells transfected by mouse anti-PD1 and human anti-PD1 expression plasmids. Data is shown in pg/cell/day. FIG. 11A shows a schematic of anti-PD1 antibody expression plasmids. CAG: CMV enhancer, anti-PD1 LC: light chain, IRES: internal ribosome entry site, anti-PD1 HC: heavy chain. FIG. 11B shows ARPE-19 cells transfected with mouse anti-PD1 vectors (mDX400). Antibody concentration in media were assayed via ELISA 24 hours later (n=3). FIG. 11C shows ARPE-19 cells transfected with human anti-PD1 vectors (Pembrolizumab). Antibody concentration in media were assayed with ELISA 24 hours later (n=3).

FIGS. 12A-D show local delivery of both IL-12 and anti-PD1 antibody in mouse. FIG. 12A shows RPE-mIL 12-ks capsules and Pembrolizumab producing capsules implanted in the IP space of Balb/C mice. FIG. 12B shows human IgG concentration in serum in Group A (Male, n=5) and Group B (Female, n=5) were measured every 5 days after treatment via ELISAs. FIG. 12C shows in vivo mouse IL-12 concentration in both IP fluid and blood after 15 days were measured via ELISAs. FIG. 12D shows in vivo Pembrolizumab concentration in both IP fluid and blood after 15 days were measured via ELISAs.

### DETAILED DESCRIPTION

The present disclosure features compositions for delivery of native human cytokines to a subject in a controlled release manner, and related methods of use thereof. These embodiments will be described below in more detail.

### A. Definitions

“Cell,” as used herein, refers to an individual cell. In some embodiments, a cell is a primary cell or is derived from a cell culture. In some embodiments, a cell is a stem cell or is derived from a stem cell. A cell may be xenogeneic, autologous, or allogeneic. In some embodiments, a cell is engineered (e.g., genetically engineered) or is not engineered (e.g., not genetically engineered). In some embodiments, the cell is an APRE-19 cell. In some embodiments, the cell expresses native human IL-12 protein.

“Prevention,” “prevent,” and “preventing” as used herein refers to a treatment that comprises administering or applying a therapy, e.g., administering a composition (e.g., as described herein) comprising a therapeutic agent (e.g., a therapeutic agent described herein) prior to the onset of a disease or condition in order to preclude the physical manifestation of said disease or condition. In some embodiments, “prevention,” “prevent,” and “preventing” require that signs or symptoms of the disease or condition have not yet developed or have not yet been observed. In some embodiments, treatment comprises prevention and in other embodiments it does not. In some embodiments, the prevention is the prevention of the recurrence of a disease, such as a tumor (cancer) after the tumor or cancer has been eradicated by an initial treatment.

“Subject,” as used herein, refers to the recipient of the composition described herein. The subject may include a human and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development (e.g., a male or female of any age group, e.g., a pediatric subject (e.g., infant, child, adolescent) or adult subject (e.g., young adult, middle-aged adult, or senior adult). A non-human animal may be a transgenic animal.

“Treatment,” “treat,” and “treating,” as used herein, refer to reversing, alleviating, delaying the onset of, or inhibiting the progress of one or more of a symptom, manifestation, or underlying cause of a disease or condition. (e.g., as described herein), e.g., by administering or applying a therapy, e.g., administering a composition comprising a therapeutic agent (e.g., a therapeutic agent described herein). In some embodiments, treating comprises reducing, reversing, alleviating, delaying the onset of, or inhibiting the progress of a symptom of a disease, disorder, or condition. In some embodiments, treating comprises reducing, reversing, alleviating, delaying the onset of, or inhibiting the progress of a manifestation of a disease or condition. In some embodiments, treating comprises reducing, reversing, alleviating, reducing, or delaying the onset of, an underlying cause of a disease or condition. In some embodiments, “treatment,” “treat,” and “treating” require that signs or symptoms of the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

“Composition,” as used herein, refers to a composition of any cell, population of cells, or plurality of cells described herein. The term “composition,” as used herein, also refers to an implantable construction comprising any cell, population of cells, or plurality of cells described herein.

### B. Cells

The composition described herein may comprise a cell, for example, an encapsulated cell. The cell may be derived from any mammalian organ or tissue, including the brain, nerves, ganglia, spine, eye, heart, liver, kidney, lung, spleen, bone, thymus, lymphatic system, skin, muscle, pancreas, stomach, intestine, blood, ovary, uterus, or testes. The cells may be derived from a donor (e.g., an allogeneic cell), derived from a subject (e.g., an autologous cell), or from another species (e.g., a xenogeneic cell). In some embodiments, the cell can be grown in cell culture, or prepared from an established cell culture line, or derived from a donor (e.g., a living donor or a cadaver).

In some embodiments, the cell may be genetically engineered. A cell may include a stem cell, such as a reprogrammed stem cell, or an induced pluripotent cell. Exemplary cells include mesenchymal stem cells (MSCs), fibroblasts (e.g., primary fibroblasts and BJ fibroblasts), human embryonic kidney (HEK) cells (e.g., HEK293T), Jurkat cells, HeLa cells, retinal pigment epithelial (RPE) cells (e.g., ARPE-19), human umbilical vein endothelial cells (HUVEC), NIH3T3 cells, Chinese hamster ovary (CHO) cells (e.g., CHO-K1), COS-1 cells, COS-7 cells, PC-3 cells, HCT 116 cells, A549MCF-7 cells, HuH-7 cells, U-2 OS cells, HepG2 cells, Neuro-2a cells, SF9 cells, human mammary epithelial cells (e.g., MCF-10a and MCF-7), NIH/3T3 cells, and human renal mix epithelial cells (HREC). In some embodiments, the cell is an RPE cell. In some embodiments, the cell is an ARPE-19 cell.

In some embodiments, the cell may produce or secrete one or more proteins of interest. In some embodiments, the cell may produce or secrete a single type of protein of interest or more than one type of protein of interest. In some embodiments may be transduced or transfected with
a nucleic acid (e.g., a vector) comprising an expression sequence of a therapeutic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the protein of interest and/or to direct targeting or secretion, for example, a promoter sequence, an activator sequence, or a cell-signaling peptide, or a cell export peptide. Exemplary promoters include EF-1a, CMV, Ube, hPGK, VMD2, and CAG.

In some embodiments described herein, the composition of encapsulated cells may comprise a cell or a plurality of cells. In the case of a plurality of cells, the concentration and total cell number may be varied depending on a number of factors, such as cell type, implantation location, and expected lifetime of the encapsulated cell composition. In an embodiment, the total number of cells included in an encapsulated cell composition is greater than about 2, 4, 6, 8, 10, 20, 30, 40, 50, 75, 100, 200, 250, 500, 750, 1000, 1500, 2000, 5000, 10000, 20000, 40000, 80000, or more. In some embodiment, the total number of cells included in a composition is greater than about 1.0×10², 1.0×10³, 1.0×10⁴, 1.0×10⁵, 1.0×10⁶, 1.0×10⁷, 1.0×10⁸, 1.0×10⁹, 1.0×10¹⁰, or more. In some embodiment, the total number of cells included in a composition is less than about than about 80000, 40000, 20000, 10000, 5000, 2500, 2000, 1500, 1000, 750, 500, 250, 200, 100, 75, 50, 40, 30, 20, 10, 8, 6, 4, 2, or less. In some embodiment, the total number of cells included in a composition is less than about 1.0×10¹⁰, 1.0×10⁹, 1.0×10⁸, 1.0×10⁷, 1.0×10⁶, 1.0×10⁵, 1.0×10⁴, 1.0×10³, 1.0×10², or less. In some embodiments, the plurality of cells is present as an aggregate or as a cell dispersion.

Specific features of a cell contained within an encapsulated cell composition may be determined, e.g., prior to and/or after incorporation into the composition. For example, cell viability, cell density, or cell expression level may be assessed. In some embodiments, cell viability, cell density, and cell expression level may be determined using standard techniques, such as cell microscopy, fluorescence microscopy, histology, or biochemical assay.

### C. Proteins of Interest

In some embodiments, the composition comprises encapsulated cells producing or secreting a protein. The protein may be of any size, e.g., greater than about 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein produced or secreted by a cell may be modified, for example, by glycosylation, methylation, or other known natural or synthetic protein modification. A protein may be produced or secreted as a pre-protein or in an inactive form and may require further modification to convert it into an active form.

In some embodiments, the cell included in the composition may produce or secrete a single type of protein or a plurality of proteins. In some embodiments, the composition may comprise a cell that is transduced or transfected with a nucleic acid (e.g., a vector) comprising an expression sequence of a therapeutic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In some embodiments, a nucleic acid introduced into a cell (e.g., as part of a plasmid) may include a region to enhance expression of the therapeutic agent and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG. Exemplary activators include the TET1 catalytic domain, P300 core, VPR, rTETR, Cas9 (e.g., from S. pyogenes or S. aureus), and Cpf1 (e.g., from L. bacterium).

In some embodiments, the protein of interest is a cytokine. In some embodiments, the cytokine is selected from the group consisting of: IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-β, IFN-γ, TNF-α, TNF-b, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, TRAIL, SCF, M-CSF, MSP (membrane scaffold protein), 4-1BBL, LIF (Leukemia Inhibitory Factor protein), OSM (Oncostatin M), or any combination thereof. In some embodiments, the protein of interest is IFN-α, IL-2, IL-12, IL-15, or any combination thereof. In some embodiments, the cytokine is IL-1. In some embodiments, the cytokine is IL-1a. In some embodiments, the cytokine is IL-1b. In some embodiments, the cytokine is IL-1RA. In some embodiments, the cytokine is IL-2. In some embodiments, the cytokine is IL-4. In some embodiments, the cytokine is IL-5. In some embodiments, the cytokine is IL-6. In some embodiments, the cytokine is IL-7. In some embodiments, the cytokine is IL-8. In some embodiments, the cytokine is IL-9. In some embodiments, the cytokine is IL-10. In some embodiments, the cytokine is IL-11. In some embodiments, the cytokine is IL-12. In some embodiments, the cytokine is IL-12a. In some embodiments, the cytokine is IL-12b. In some embodiments, the cytokine is IL-13. In some embodiments, the cytokine is IL-14. In some embodiments, the cytokine is IL-15. In some embodiments, the cytokine is IL-16. In some embodiments, the cytokine is IL-17. In some embodiments, the cytokine is IL-20. In some embodiments, the cytokine is IFN-α. In some embodiments, the cytokine is IFN-β. In some embodiments, the cytokine is IFN-γ. In some embodiments, the cytokine is TNF-α. In some embodiments, the cytokine is TNF-b. In some embodiments, the cytokine is TGF-b. In some embodiments, the cytokine is CCL-1. In some embodiments, the cytokine is CCL-2. In some embodiments, the cytokine is CCL-3. In some embodiments, the cytokine is CCL-4. In some embodiments, the cytokine is CCL-5. In some embodiments, the cytokine is CCL-6. In some embodiments, the cytokine is CCL-7. In some embodiments, the cytokine is CCL-8. In some embodiments, the cytokine is CCL-9. In some embodiments, the cytokine is CCL-10. In some embodiments, the cytokine is CCL-11. In some embodiments, the cytokine is CCL-12. In some embodiments, the cytokine is CCL-13. In some embodiments, the cytokine is CCL-14. In some embodiments, the cytokine is CCL-15. In some embodiments, the cytokine is CCL-16. In some embodiments, the cytokine is CCL-17. In some embodiments, the cytokine is CCL-18. In some embodiments, the cytokine is CCL-19. In some embodiments, the cytokine is CCL-20. In some embodiments, the cytokine is CCL-21. In some embodiments, the cytokine is CCL-22. In some embodiments, the cytokine is CCL-23. In some embodiments, the cytokine is CCL-24. In some embodiments, the cytokine is CCL-25. In some embodiments, the cytokine is CCL-26. In some embodiments, the cytokine is CCL-27. In some embodiments, the cytokine is CCL-28. In some embodiments, the cytokine is CXCL1. In some embodiments, the cytokine is CXCL2. In some embodiments, the cytokine is CXCL3. In some embodiments, the cytokine is CXCL4. In some embodiments, the cytokine is CXCL5. In some embodiments, the cytokine is CXCL6. In some embodiments, the cytokine is CXCL7. In some embodiments, the cytokine is CXCL8. In some embodiments, the cytokine is CXCL9. In some embodiments, the cytokine is CXCL10. In some embodiments, the cytokine is CXCL11. In some embodiments, the cytokine is CXCL12. In some embodiments, the cytokine is CXCL13. In some embodiments, the cytokine is CXCL14. In some embodiments, the cytokine is CXCL15. In some embodiments, the cytokine is CXCL16. In some embodiments, the cytokine is CXCL17. In some embodiments, the cytokine is TRAIL. In some embodiments, the cytokine is SCF. In some embodiments, the cytokine is M-CSF. In some embodiments, the cytokine is MSP (membrane scaffold protein). In some embodiments, the cytokine is 4-1BBL. In some embodiments, the cytokine is LIF (Leukemia Inhibitory Factor protein). In some embodiments, the cytokine is OSM (Oncostatin M).

In some embodiments, the therapeutic agent may be a pro-inflammatory cytokine, such as IL-2, IL-12, IL-15, IFN-α, IFN-γ, or TNF-α. Nucleic acid sequences encoding exemplary cytokines are as shown below in Table 1.

TABLE 1

| Cytokine | Sequence |
|---|---|
| mIL-2 | ATGTACAGCATGCAGCTCGCATCCTGTGTCACATTGACACTTGTGCTCCTTGTCAACAGCGCAC |
| | CCACTTCAAGCTCCACTTCAAGCTCTACAGCGGAAGCACAGCAGCAGCAGCAGCAGCAGCAGCA |
| | GCAGCAGCAGCACCTGGAGCAGCTGTTGATGGACCTACAGGAGCTCCTGAGCAGGATGGAGAAT |
| | TACAGGAACCTGAAACTCCCCAGGATGCTCACCTTCAAATTTTACTTGCCCAAGCAGGCCACAG |
| | AATTGAAAGATCTTCAGTGCCTAGAAGATGAACTTGGACCTCTGCGGCATGTTCTGGATTTGAC |
| | TCAAAGCAAAAGCTTTCAATTGGAAGATGCTGAGAATTTCATCAGCAATATCAGAGTAACTGTT |
| | GTAAAACTAAAGGGCTCTGACAACACATTTGAGTGCCAATTCGATGATGAGTCAGCAACTGTGG |
| | TGGACTTTCTGAGGAGATGGATAGCCTTCTGTCAAAGCATCATCTCAACAAGCCCTCAATAA |
| | (SEQ ID NO: 24) |
| mIL-12a | ATGGTCAGCGTTCCAACAGCCTCACCCTCGGCATCCAGCAGCTCCTCTCAGTGCCGGTCCAGCA |
| | TGTGTCAATCACGCTACCTCCTCTTTTTGGCCACCCTTGCCCTCCTAAACCACCTCAGTTTGGC |
| | CAGGGTCATTCCAGTCTCTGGACCTGCCAGGTGTCTTAGCCAGTCCCGAAACCTGCTGAAGACC |
| | ACAGATGACATGGTGAAGACGGCCAGAGAAAAACTGAAACATTATTCCTGCACTGCTGAAGACA |
| | TCGATCATGAAGACATCACACGGGACCAAACCAGCACATTGAAGACCTGTTTACCACTGGAACT |
| | ACACAAGAACGAGAGTTGCCTGGCTACTAGAGAGACTTCTTCCACAACAAGAGGGAGCTGCCTG |
| | CCCCCACAGAAGACGTCTTTGATGATGACCCTGTGCCTTGGTAGCATCTATGAGGACTTGAAGA |
| | TGTACCAGACAGAGTTCCAGGCCATCAACGCAGCACTTCAGAATCACAACCATCAGCAGATCAT |
| | TCTAGACAAGGGCATGCTGGTGGCCATCGATGAGCTGATGCAGTCTCTGAATCATAATGGCGAG |
| | ACTCTGCGCCAGAAACCTCCTGTGGGAGAAGCAGACCCTTACAGAGTGAAAATGAAGCTCTGCA |
| | TCCTGCTTCACGCCTTCAGCACCCGCGTCGTGACCATCAACAGGGTGATGGGCTATCTGAGCTC |
| | CGCCTGA (SEQ ID NO: 25) |
| mIL-12b | ATGTGTCCTCAGAAGCTAACCATCTCCTGGTTTGCCATCGTTTTGCTGGTGTCTCCACTCATGG |
| | CCATGTGGGAGCTGGAGAAAGACGTTTATGTTGTAGAGGTGGACTGGACTCCCGATGCCCCTGG |
| | AGAAACAGTGAACCTCACCTGTGACACGCCTGAAGAAGATGACATCACCTGGACCTCAGACCAG |
| | AGACATGGAGTCATAGGCTCTGGAAAGACCCTGACCATCACTGTCAAAGAGTTTCTAGATGCTG |
| | GCCAGTACACCTGCCACAAAGGAGGCGAGACTCTGAGCCACTCACATCTGCTGCTCCACAAGAA |
| | GGAAAATGGAATTTGGTCCACTGAAATTTTAAAAAATTTCAAAAACAAGACTTTCCTGAAGTGT |
| | GAAGCACCAAATTACTCCGGACGGTTCACGTGCTCATGGCTGGTGCAAAGAAACATGGACTTGA |
| | AGTTCAACATCAAGAGCAGTAGCAGTTCCCCTGACTCTCGGGCAGTGACATGTGGAATGGCGTC |
| | TCTGTCTGCAGAGAAGGTCACACTGGACCAAAGGGACTATGAGAAGTATTCAGTGTCCTGCCAG |
| | GAGGATGTCACCTGCCCAACTGCCGAGGAGACCCTGCCCATTGAACTGGCGTTGGAAGCACGGC |
| | AGCAGAATAAATATGAGAACTACAGCACCAGCTTCTTCATCAGGGACATCATCAAACCAGACCC |
| | GCCCAAGAACTTGCAGATGAAGCCTTTGAAGAACTCACAGGTGGAGGTCAGCTGGGAGTACCCT |
| | GACTCCTGGAGCACTCCCCATTCCTACTTCTCCCTCAAGTTCTTTGTTCGAATCCAGCGCAAGA |
| | AAGAAAAGATGAAGGAGACAGAGGAGGGGTGTAACCAGAAAGGTGCGTTCCTCGTAGAGAAGAC |
| | ATCTACCGAAGTCCAATGCAAAGGCGGGAATGTCTGCGTGCAAGCTCAGGATCGCTATTACAAT |
| | TCCTCATGCAGCAAGTGGGCATGTGTTCCCTGCAGGGTCCGATCCTAG (SEQ ID NO: 26) |
| mIL-15 | ATGAAAATTTTGAAACCATATATGAGGAATACATCCATCTCGTGCTACTTGTGTTTCCTTCTAA |
| | ACAGTCACTTTTTAACTGAGGCTGGCATTCATGTCTTCATTTTGGGCTGTGTCAGTGTAGGTCT |
| | CCCTAAAACAGAGGCCAACTGGATAGATGTAAGATATGACCTGGAGAAAATTGAAAGCCTTATT |
| | CAATCTATTCATATTGACACCACTTTATACACTGACAGTGACTTTCATCCCAGTTGCAAAGTTA |
| | CTGCAATGAACTGCTTTCTCCTGGAATTGCAGGTTATTTTACATGAGTACAGTAACATGACTCT |
| | TAATGAAACAGTAAGAAACGTGCTCTACCTTGCAAACAGCACTCTGTCTTCTAACAAGAATGTA |
| | GCAGAATCTGGCTGCAAGGAATGTGAGGAGCTGGAGGAGAAAACCTTCACAGAGTTTTTGCAAA |
| | GCTTTATACGCATTGTCCAAATGTTCATCAACACGTCCTGA (SEQ ID NO: 14) |
| mIFN-α | ATGGCTAGGCTCTGTGCTTTCCTCATGATCCTGGTAATGATGAGCTACTACTGGTCAGCCTGTT |
| | CTCTAGGATGTGACCTGCCTCACACTTATAACCTCGGGAACAAGAGGGCCTTGACAGTCCTGGA |
| | AGAAATGAGAAGACTCCCCCCTCTTTCCTGCCTGAAGGACAGGAAGGATTTTGGATTCCCCTTG |
| | GAGAAGGTGGATAACCAACAGATCCAGAAGGCTCAAGCCATCCTTGTGCTAAGAGATCTTACCC |
| | AGCAGATTTTGAACCTCTTCACATCAAAAGACTTGTCTGCTACTTGGAATGCAACTCTACTAGA |
| | CTCATTCTGCAATGACCTCCATCAGCAGCTCAATGACCTCAAAGCCTGTGTGATGCAGGAACCT |
| | CCTCTGACCCAGGAAGACTCCCTGCTGGCTGTGAGGACATACTTCCACAGGATCACTGTGTACC |
| | TGAGAAAGAAGAAACACAGCCTCTGTGCCTGGGAGGTGATCAGAGCAGAAGTCTGGAGAGCCCT |
| | CTCTTCCTCAACCAACTTGCTGGCAAGACTGAGTGAGGAGAAGGAGTGA (SEQ ID NO: |
| | 15) |
| mIFN-γ | ATGAACGCTACACACTGCATCTTGGCTTTGCAGCTCTTCCTCATGGCTGTTTCTGGCTGTTACT |
| | GCCACGGCACAGTCATTGAAAGCCTAGAAAGTCTGAATAACTATTTTAACTCAAGTGGCATAGA |
| | TGTGGAAGAAAAGAGTCTCTTCTTGGATATCTGGAGGAACTGGCAAAAGGATGGTGACATGAAA |
| | ATCCTGCAGAGCCAGATTATCTCTTTCTACCTCAGACTCTTTGAAGTCTTGAAAGACAATCAGG |
| | CCATCAGCAACAACATAAGCGTCATTGAATCACACCTGATTACTACCTTCTTCAGCAACAGCAA |
| | GGCGAAAAAGGATGCATTCATGAGTATTGCCAAGTTTGAGGTCAACAACCCACAGGTCCAGCGC |
| | CAAGCATTCAATGAGCTCATCCGAGTGGTCCACCAGCTGTTGCCGGAATCCAGCCTCAGGAAGC |
| | GGAAAAGGAGTCGCTGCTGA (SEQ ID NO: 16) |
| mTNF-α | ATGAGCACAGAAAGCATGATCCGCGACGTGGAACTGGCAGAAGAGGCACTCCCCCAAAAGATGG |
| | GGGGCTTCCAGAACTCCAGGCGGTGCCTATGTCTCAGCCTCTTCTCATTCCTGCTTGTGGCAGG |
| | GGCCACCACGCTCTTCTGTCTACTGAACTTCGGGGTGATCGGTCCCCAAAGGGATGAGAAGTTC |
| | CCAAATGGCCTCCCTCTCATCAGTTCTATGGCCCAGACCCTCACACTCAGATCATCTTCTCAAA |
| | ATTCGAGTGACAAGCCTGTAGCCCACGTCGTAGCAAACCACCAAGTGGAGGAGCAGCTGGAGTG |
| | GCTGAGCCAGCGCGCCAACGCCCTCCTGGCCAACGGCATGGATCTCAAAGACAACCAACTAGTG |
| | GTGCCAGCCGATGGGTTGTACCTTGTCTACTCCCAGGTTCTCTTCAAGGGACAAGGCTGCCCCG |
| | ACTACGTGCTCCTCACCCACACCGTCAGCCGATTTGCTATCTCATACCAGGAGAAAGTCAACCT |
| | CCTCTCTGCCGTCAAGAGCCCCTGCCCCAAGGACACCCCTGAGGGGGCTGAGCTCAAACCCTGG |
| | TATGAGCCCATATACCTGGGAGGAGTCTTCCAGCTGGAGAAGGGGGACCAACTCAGCGCTGAGG |
| | TCAATCTGCCCAAGTACTTAGACTTTGCGGAGTCCGGGCAGGTCTACTTTGGAGTCATTGCTCT |
| | GTGA (SEQ ID NO: 17) |

In some embodiments, the therapeutic agent is an IL-2. In some embodiments, the IL-2 is encoded by the nucleic acid sequence of SEQ ID NO: 24. In some embodiments, the therapeutic agent is a native human IL-2. In some embodiments, the cytokine is a recombinant IL-2. In some embodiments, native human IL-2 refers to a protein encoded by a nucleic acid sequence comprising:

(SEQ ID NO: 1)
ATGTACAGGATGCAACTCCTGTCTTGCATTGCACTAAGTCTTGCACTTGT
CACAAACAGTGCACCTACTTCAAGTTCTACAAAGAAAACACAGCTACAAC
TGGAGCATTTACTGCTGGATTTACAGATGATTTTGAATGGAATTAATAAT
TACAAGAATCCCAAACTCACCAGGATGCTCACATTTAAGTTTTACATGCC
CAAGAAGGCCACAGAACTGAAACATCTTCAGTGTCTAGAAGAAGAACTCA
AACCTCTGGAGGAAGTGCTAAATTTAGCTCAAAGCAAAAACTTTCACTTA
AGACCCAGGGACTTAATCAGCAATATCAACGTAATAGTTCTGGAACTAAA
GGGATCTGAAACAACATTCATGTGTGAATATGCTGATGAGACAGCAACCA
TTGTAGAATTTCTGAACAGATGGATTACCTTTTGTCAAAGCATCATCTCA
ACACTGACTTGA.

In some embodiments, the nucleic acid coding sequence encoding native human IL-2 is codon-optimized. In some embodiments, the nucleic acid coding sequence encoding native human IL-2 is codon-optimized for expression in a mammalian cell. The codon optimized sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, http://www.jcat.de, Grote, A. et al., Nucleic Acids Research, Vol 33, Issue suppl_2, pp. W526-W531 (2005), IDT Codon Optimization Tool (Integrated DNA Technologies), VectorBuilder Codon Optimization tool (VectorBuilder Inc.), Codon Optimization OnLine (COOL, http://bioinfo.bti.a-star.edu.sg/COOL/; Chin J. X., et al., Bioinformatics, Vol 30, Issue 15, p. 2210-2212 (2014)), or ExpOptimizer (NovoPro, Shanghai, China). Examples of codon-optimized nucleic acid coding sequences encoding native human IL-2 comprise, but are not limited to:

(SEQ ID NO: 2)
ATGTACCGGATGCAGCTGCTGTCCTGCATCGCACTGTCCCTCGCCCTGGT
GACAAATTCTGCCCCCACCTCCTCCAGCACAAAAAAGACCCAGTTGCAGC
TGGAGCACCTGCTGCTGGATCTGCAGATGATCCTGAATGGCATCAATAAC
TACAAAAACCCTAAACTGACCAGAATGCTGACCTTTAAATTTTACATGCC
TAAAAAGGCAACCGAGCTGAAGCACCTGCAGTGCCTGGAAGAGGAACTGA
AGCCCCTGGAGGAGGTGCTGAACCTGGCCCAGAGCAAGAACTTTCACCTG
CGGCCCCGCGACCTGATCAGCAACATCAACGTGATCGTGCTGGAGCTGAA
GGGCAGTGAAACCACATTCATGTGCGAGTACGCCGACGAGACCGCCACAA
TCGTGGAGTTCCTGAACAGATGGATCACATTCTGTCAGTCCATCATTAGC
ACACTGACCTAA;
(SEQ ID NO: 3)
ATGTACCGCATGCAGCTGCTGAGCTGCATCGCCCTGAGCCTGGCCCTGGT
GACCAACAGCGCCCCCACCAGCAGCAGCACCAAGAAGACCCAGCTGCAGC
TGGAGCACCTGCTGCTGGACCTGCAGATGATCCTGAACGGCATCAACAAC
TACAAGAACCCCAAGCTGACCCGCATGCTGACCTTCAAGTTCTACATGCC
CAAGAAGGCCACCGAGCTGAAGCACCTGCAGTGCCTGGAGGAGGAGCTGA
AGCCCCTGGAGGAGGTGCTGAACCTGGCCCAGAGCAAGAACTTCCACCTG
CGCCCCCGCGACCTGATCAGCAACATCAACGTGATCGTGCTGGAGCTGAA
GGGCAGCGAGACCACCTTCATGTGCGAGTACGCCGACGAGACCGCCACCA
TCGTGGAGTTCCTGAACCGCTGGATCACCTTCTGCCAGAGCATCATCAGC
ACCCTGACCTAA;
(SEQ ID NO: 4)
ATGTATAGGATGCAGCTGCTCTCTTGTATCGCGTTGTCTCTGGCTTTGGT
GACTAACTCAGCTCCCACGTCCAGCAGTACCAAAAAGACCCAGCTGCAGC
TGGAACATCTTCTGTTGGATCTGCAAATGATACTGAATGGGATCAACAAC
TATAAAAACCCAAAACTGACTAGAATGCTGACTTTCAAGTTCTACATGCC
TAAAAAGGCAACAGAATTGAAGCACCTTCAGTGCCTGGAGGAGGAGCTTA
AGCCCCTGGAGGAGGTGCTGAATCTGGCCCAAAGTAAGAATTTTCATCTG
CGACCCAGGGATCTGATCAGTAATATCAATGTGATCGTCCTGGAGCTGAA
GGGCAGTGAGACCACGTTTATGTGTGAATACGCAGACGAAACCGCCACTA
TCGTTGAATTCTTGAACAGGTGGATCACCTTTTGTCAGAGTATCATCAGC
ACCCTCACT;
or
(SEQ ID NO: 5)
ATGTACAGAATGCAGCTGCTGAGCTGCATCGCCCTGAGCCTGGCCCTGGT
GACCAACAGCGCCCCCACAAGCAGCAGCACCAAGAAGACACAGCTGCAGC
TGGAGCACCTGCTGCTGGACCTGCAGATGATCCTGAACGGCATCAACAAC
TACAAGAACCCCAAGCTGACAAGAATGCTGACCTTCAAGTTCTACATGCC
CAAGAAGGCCACCGAGCTGAAGCACCTGCAGTGCCTGGAGGAGGAGCTGA
AGCCCCTGGAAGAGGTGCTGAACCTGGCTCAGAGCAAGAACTTCCACCTG
AGACCTAGAGACCTGATCAGCAACATCAACGTGATCGTGCTGGAGCTGAA
GGGCAGCGAGACCACCTTCATGTGCGAGTACGCCGACGAGACCGCCACCA
TCGTGGAGTTCCTGAACAGATGGATCACCTTCTGTCAGAGCATCATCAGC
ACCCTGACCTGA.

In some embodiments, the codon-optimized nucleic acid coding sequence encoding native human IL-2 comprise the nucleic acid sequence as set forth in SEQ ID NO: 2-5. In some embodiments, the codon-optimized nucleic acid coding sequence encoding native human IL-2 comprise the nucleic acid sequence as set forth in SEQ ID NO: 2. In some embodiments, the codon-optimized nucleic acid coding sequence encoding native human IL-2 comprise the nucleic acid sequence having at least 90%, 91%, 92%, 93%, 94%, 95%, 96%, 97%, 98%, 99%, or 100% identity to the sequence of SEQ ID NO: 2-5. In some embodiments, the codon-optimized nucleic acid coding sequence encoding native human IL-2 comprise the nucleic acid sequence having at least 90%, 91%, 92%, 93%, 94%, 95%, 96%, 97%, 98%, 99%, or 100% identity to the sequence of SEQ ID NO: 2.

In some embodiments, the native human IL-2 protein produced by the cell is formed from the formed from an amino acid sequence of:

(SEQ ID NO: 6)
MYRMQLLSCIALSLALVINSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTEMCEYADETATIVEFLNRWITFCQSIIS
TLT

Without being bound by any particular theory, the native IL-2 produced by the cells comprising the nucleic acid sequence of SEQ ID NO: 1 differs from recombinant IL-2 produced from bacteria or other non-eukaryotic cells due to differences in post-translational modification. Thus, the cells expressing the native human IL-2 is superior because it has superior potency. This is described herein in the Examples section. In some embodiments, the IL-2 is a IL-2 mutein or a modified IL-2 molecule, fusion proteins or antibodies that act on the IL-2 pathway. In some embodiments, the IL-2 is a pegylated IL-2 molecule. In some embodiments, the pegylated IL-2 molecule has a wild-type sequence. In some embodiments, the pegylated IL-2 has a mutant IL-2 sequence. In some embodiments, the capsules or cells are used to produce NKTR-214 (pegylated IL-2; Clin Cancer Res Feb. 1, 2016 (22) (3) 680-690; DOI: 10.1158/1078-0432.CCR-15-1631, which is hereby incorporated by reference in its entirety), THOR-707 (SAR444245; Annals of Oncology, Volume 30, Supplement 5, October 2019, Page v501,, which is hereby incorporated by reference in its entirety), ALKS 4230 (J Immunother Cancer. 2020 April; 8 (1): e000673. doi: 10.1136/jitc-2020-000673, Nemvaleukin Alfa), TransCon IL-2 β/γ, BNT151, BNT153, CLN-617, CUE-101, CUE-102, CUE-103, Anktiva® (N-803), KY1043, MDNA11, NL-201, SO-C101, RO6874281, Simlukafusp Alfa, RG7461, WTX-124, WTX-330, XTX202, or XTX401 or any combination thereof. In some embodiments, the IL-2 mutein comprises an amino acid sequence having at least 75%, 80%, 85%, 90%, 91%, 92%, 93%, 94%, 95%, 96%, 97%, 98%, 99%, or 100% identity to the amino acid sequence of SEQ ID NO: 6.

In some embodiments, the cytokine is a native human IL-12. In some embodiments, the cytokine is a recombinant IL-12. In some embodiments, the cytokine is an IL-12 mutein. In some embodiments, the cytokine is a native human IL-12a. In some embodiments, the cytokine is a native human IL-12b. In some embodiments, the cytokine is an IL-12a mutein. In some embodiments, the cytokine is an IL-12b mutein. In some embodiments, the cytokine is a recombinant IL-12a. In some embodiments, the cytokine is a recombinant IL-12b. In some embodiments, the cytokine is a recombinant IL-12. In some embodiments, the cytokine is an IL-12 mutein. In some embodiments, a composition described herein may contain a plurality of cytokines, for example, produced or secreted by a population of cells, such as native human IL-12a and native human IL-12b. In some embodiments, the composition comprises a cell or a plurality of cells that are genetically engineered to produce or secrete a cytokine. In some embodiments, the composition comprises a cell or a plurality of cells that are genetically engineered to produce or secrete a plurality of cytokines.

In some embodiments, IL-12 refers to a IL-12 (p70), or a heterodimeric complex comprising IL-12a and IL-12b. In some embodiments, IL-12 refers to a IL-12 (p70), or a heterodimeric complex comprising mouse IL-12a and mouse IL-12b. In some embodiments, native human IL-12 refers to a IL-12 (p70), or a heterodimeric complex comprising native human IL-12a and native human IL-12b. Without wishing to be bound to a particular theory, when the IL-12a subunit and the IL-12b subunit are expressed, they interact with each other to form a heterodimeric complex comprising the IL-12 (p70), or as referred to herein, IL-12. In some embodiments, IL-12a refers to a protein encoded by a nucleic acid sequence of SEQ ID NO: 25. In some embodiments, mouse IL-12a refers to a protein encoded by a nucleic acid sequence of SEQ ID NO: 25. In some embodiments, native human IL-12a refers to a protein encoded by a nucleic acid sequence comprising:

(SEQ ID NO: 7)
ATTTCGCTTTCATTTTGGGCCGAGCTGGAGGCGGCGGGGCCGTCCCGGAA
CGGCTGCGGCCGGGCACCCCGGGAGTTAATCCGAAAGCGCCGCAAGCCCC
GCGGGCCGGCCGCACCGCACGTGTCACCGAGAAGCTGATGTAGAGAGAGA
CACAGAAGGAGACAGAAAGCAAGAGACCAGAGTCCCGGGAAAGTCCTGCC
GCGCCTCGGGACAATTATAAAAATGTGGCCCCCTGGGTCAGCCTCCCAGC
CACCGCCCTCACCTGCCGCGGCCACAGGTCTGCATCCAGCGGCTCGCCCT
GTGTCCCTGCAGTGCCGGCTCAGCATGTGTCCAGCGCGCAGCCTCCTCCT
TGTGGCTACCCTGGTCCTCCTGGACCACCTCAGTTTGGCCAGAAACCTCC
CCGTGGCCACTCCAGACCCAGGAATGTTCCCATGCCTTCACCACTCCCAA
AACCTGCTGAGGGCCGTCAGCAACATGCTCCAGAAGGCCAGACAAACTCT
AGAATTTTACCCTTGCACTTCTGAAGAGATTGATCATGAAGATATCACAA
AAGATAAAACCAGCACAGTGGAGGCCTGTTTACCATTGGAATTAACCAAG
AATGAGAGTTGCCTAAATTCCAGAGAGACCTCTTTCATAACTAATGGGAG
TTGCCTGGCCTCCAGAAAGACCTCTTTTATGATGGCCCTGTGCCTTAGTA
GTATTTATGAAGACTTGAAGATGTACCAGGTGGAGTTCAAGACCATGAAT
GCAAAGCTTCTGATGGATCCTAAGAGGCAGATCTTTCTAGATCAAAACAT
GCTGGCAGTTATTGATGAGCTGATGCAGGCCCTGAATTTCAACAGTGAGA
CTGTGCCACAAAAATCCTCCCTTGAAGAACCGGATTTTTATAAAACTAAA
ATCAAGCTCTGCATACTTCTTCATGCTTTCAGAATTCGGGCAGTGACTAT
TGATAGAGTGATGAGCTATCTGAATGCTTCCTAAAAAGCGAGGTCCCTCC
AAACCGTTGTCATTTTTATAAAACTTTGAAATGAGGAAACTTTGATAGGA
TGTGGATTAAGAACTAGGGAGGGGGAAAGAAGGATGGGACTATTACATCC
ACATGATACCTCTGATCAAGTATTTTTGACATTTACTGTGGATAAATTGT
TTTTAAGTTTTCATGAATGAATTGCTAAGAAGGGAAAATATCCATCCTGA
AGGTGTTTTTCATTCACTTTAATAGAAGGGCAAATATTTATAAGCTATTT
CTGTACCAAAGTGTTTGTGGAAACAAACATGTAAGCATAACTTATTTTAA
AATATTTATTTATATAACTTGGTAATCATGAAAGCATCTGAGCTAACTTA
TATTTATTTATGTTATATTTATTAAATTATTTATCAAGTGTATTTGAAAA
ATATTTTTAAGTGTTCTAAAAATAAAAGTATTGAATTAAAGTGA.

In some embodiments, IL-12b refers to a protein encoded by a nucleic acid sequence of SEQ ID NO: 26. In some embodiments, mouse IL-12b refers to a protein encoded by a nucleic acid sequence of SEQ ID NO: 26. In some embodiments, native human IL-12b refers to a protein encoded by a nucleic acid sequence comprising:

(SEQ ID NO: 8)
AGAAGAAACAACATCTGTTTCAGGGCCATTGGACTCTCCGTCCTGCCCAG
AGCAAGATGTGTCACCAGCAGTTGGTCATCTCTTGGTTTTCCCTGGTTTT
TCTGGCATCTCCCCTCGTGGCCATATGGGAACTGAAGAAAGATGTTTATG
TCGTAGAATTGGATTGGTATCCGGATGCCCCTGGAGAAATGGTGGTCCTC
ACCTGTGACACCCCTGAAGAAGATGGTATCACCTGGACCTTGGACCAGAG
CAGTGAGGTCTTAGGCTCTGGCAAAACCCTGACCATCCAAGTCAAAGAGT
TTGGAGATGCTGGCCAGTACACCTGTCACAAAGGAGGCGAGGTTCTAAGC
CATTCGCTCCTGCTGCTTCACAAAAAGGAAGATGGAATTTGGTCCACTGA
TATTTTAAAGGACCAGAAAGAACCCAAAAATAAGACCTTTCTAAGATGCG
AGGCCAAGAATTATTCTGGACGTTTCACCTGCTGGTGGCTGACGACAATC
AGTACTGATTTGACATTCAGTGTCAAAAGCAGCAGAGGCTCTTCTGACCC
CCAAGGGGTGACGTGCGGAGCTGCTACACTCTCTGCAGAGAGAGTCAGAG
GGGACAACAAGGAGTATGAGTACTCAGTGGAGTGCCAGGAGGACAGTGCC
TGCCCAGCTGCTGAGGAGAGTCTGCCCATTGAGGTCATGGTGGATGCCGT
TCACAAGCTCAAGTATGAAAACTACACCAGCAGCTTCTTCATCAGGGACA
TCATCAAACCTGACCCACCCAAGAACTTGCAGCTGAAGCCATTAAAGAAT
TCTCGGCAGGTGGAGGTCAGCTGGGAGTACCCTGACACCTGGAGTACTCC
ACATTCCTACTTCTCCCTGACATTCTGCGTTCAGGTCCAGGGCAAGAGCA
AGAGAGAAAAGAAAGATAGAGTCTTCACGGACAAGACCTCAGCCACGGTC
ATCTGCCGCAAAAATGCCAGCATTAGCGTGCGGGCCCAGGACCGCTACTA
TAGCTCATCTTGGAGCGAATGGGCATCTGTGCCCTGCAGTTAGGTTCTGA
TCCAGGATGAAAATTTGGAGGAAAAGTGGAAGATATTAAGCAAAATGTTT
AAAGACACAACGGAATAGACCCAAAAAGATAATTTCTATCTGATTTGCTT
TAAAACGTTTTTTTAGGATCACAATGATATCTTTGCTGTATTTGTATAGT
TAGATGCTAAATGCTCATTGAAACAATCAGCTAATTTATGTATAGATTTT
CCAGCTCTCAAGTTGCCATGGGCCTTCATGCTATTTAAATATTTAAGTAA
TTTATGTATTTATTAGTATATTACTGTTATTTAACGTTTGTCTGCCAGGA
TGTATGGAATGTTTCATACTCTTATGACCTGATCCATCAGGATCAGTCCC
TATTATGCAAAATGTGAATTTAATTTTATTTGTACTGACAACTTTTCAAG
CAAGGCTGCAAGTACATCAGTTTTATGACAATCAGGAAGAATGCAGTGTT
CTGATACCAGTGCCATCATACACTTGTGATGGATGGGAACGCAAGAGATA
CTTACATGGAAACCTGACAATGCAAACCTGTTGAGAAGATCCAGGAGAAC
AAGATGCTAGTTCCCATGTCTGTGAAGACTTCCTGGAGATGGTGTTGATA
AAGCAATTTAGGGCCACTTACACTTCTAAGCAAGTTTAATCTTTGGATGC
CTGAATTTTAAAAGGGCTAGAAAAAAATGATTGACCAGCCTGGGAAACAT
AACAAGACCCCGTCTCTACAAAAAAAATTTAAAATTAGCCAGGCGTGGTG
GCTCATGCTTGTGGTCCCAGCTGTTCAGGAGGATGAGGCAGGAGGATCTC
TTGAGCCCAGGAGGTCAAGGCTATGGTGAGCCGTGATTGTGCCACTGCAT
ACCAGCCTAGGTGACAGAATGAGACCCTGTCTCAAAAAAAAAAATGATTG
AAATTAAAATTCAGCTTTAGCTTCCATGGCAGTCCTCACCCCCACCTCTC
TAAAAGACACAGGAGGATGACACAGAAACACCGTAAGTGTCTGGAAGGCA
AAAAGATCTTAAGATTCAAGAGAGAGGACAAGTAGTTATGGCTAAGGACA
TGAAATTGTCAGAATGGCAGGTGGCTTCTTAACAGCCCTGTGAGAAGCAG
ACAGATGCAAAGAAAATCTGGAATCCCTTTCTCATTAGCATGAATGAACC
TGATACACAATTATGACCAGAAAATATGGCTCCATGAAGGTGCTACTTTT
AAGTAATGTATGTGCGCTCTGTAAAGTGATTACATTTGTTTCCTGTTTGT
TTATTTATTTATTTATTTTTGCATTCTGAGGCTGAACTAATAAAAACTCT
TCTTTGTAATCATA.

In some embodiments, the native human IL-12 protein produced by the cell comprises a polypeptide comprising a first polypeptide comprising the sequence of:

(SEQ ID NO: 9)
MCPARSLLLVATLVLLDHLSLARNLPVATPDPGMFPCLHHSQNLLRAVSN
MLQKARQTLEFYPCTSEEIDHEDITKDKTSTVEACLPLELTKNESCLNSR
ETSFITNGSCLASRKTSEMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS;

and a second polypeptide comprising the sequence of:

(SEQ ID NO: 10)
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCS;

wherein the first and second polypeptide forms a heterodimeric complex.

In some embodiments, the IL-12a polypeptide and the IL-12b polypeptide is expressed at a ratio of 1:1, 1:2, 1:3, 1:4, 1:5, 1:6, 1:7, 1:8, 1:9, or 1:10. In some embodiments, the IL-12a polypeptide and the IL-12b polypeptide is expressed at a ratio of 1:1. In some embodiments, the IL-12a polypeptide and the IL-12b polypeptide is expressed at a ratio of 1:2. In some embodiments, the IL-12a polypeptide and the IL-12b polypeptide is expressed at a ratio of 1:3. In some embodiments, the IL-12a polypeptide and the IL-12b polypeptide is expressed at a ratio of 1:4. In some embodiments, the IL-12a polypeptide and the IL-12b polypeptide is expressed at a ratio of 1:5. In some embodiments, the IL-12a polypeptide and the IL-12b polypeptide is expressed at a ratio of 1:6. In some embodiments, the IL-12a polypeptide and the IL-12b polypeptide is expressed at a ratio of 1:7. In some embodiments, the IL-12a polypeptide and the IL-12b polypeptide is expressed at a ratio of 1:8. In some embodiments, the IL-12a polypeptide and the IL-12b polypeptide is expressed at a ratio of 1:9. In some embodiments, the IL-12a polypeptide and the IL-12b polypeptide is expressed at a ratio of 1:10. In some embodiments, the IL-12a polypeptide and the IL-12b polypeptide expressed at a ratio of 1:1, 1:2, 1:3, 1:4, 1:5, 1:6, 1:7, 1:8, 1:9, or 1:10, interact with each other to form a heterodimeric complex. In some embodiments, the IL-12a polypeptide and the IL-12b polypeptide, expressed at a ratio of 1:1, interact with each other to form a heterodimeric complex. In some embodiments, the IL-12a polypeptide and the IL-12b polypeptide, expressed at a ratio of 1:2, interact with each other to form a heterodimeric complex. In some embodiments, the IL-12a polypeptide and the IL-12b polypeptide, expressed at a ratio of 1:3, interact with each other to form a heterodimeric complex. In some embodiments, the IL-12a polypeptide and the IL-12b polypeptide, expressed at a ratio of 1:4, interact with each other to form a heterodimeric complex. In some embodiments, the IL-12a polypeptide and the IL-12b polypeptide, expressed at a ratio of 1:5, interact with each other to form a heterodimeric complex. In some embodiments, the IL-12a polypeptide and the IL-12b polypeptide, expressed at a ratio of 1:6, interact with each other to form a heterodimeric complex. In some embodiments, the IL-12a polypeptide and the IL-12b polypeptide, expressed at a ratio of 1:7, interact with each other to form a heterodimeric complex. In some embodiments, the IL-12a polypeptide and the IL-12b polypeptide, expressed at a ratio of 1:8, interact with each other to form a heterodimeric complex. In some embodiments, the IL-12a polypeptide and the IL-12b polypeptide, expressed at a ratio of 1:9, interact with each other to form a heterodimeric complex. In some embodiments, the IL-12a polypeptide and the IL-12b polypeptide, expressed at a ratio of 1:10, interact with each other to form a heterodimeric complex.

In some embodiments, the IL-12 is a IL-12 mutein, a recombinant IL-12, or a modified IL-12 molecule, fusion proteins or antibodies that act on the IL-12 pathway. In some embodiments, the IL-12a is a IL-12a mutein, a recombinant IL-12a, or a modified IL-12a molecule, a fusion proteins or an antibody. In some embodiments, the IL-12b is a IL-12b mutein, a recombinant IL-12b, or a modified IL-12b molecule, a fusion proteins or an antibody. In some embodiments, the IL-12a and the IL-12b interact with one another to form a heterodimeric complex.

In some embodiments, an IL-12 mutein can be prepared by mutating one or more of the residues of IL-12. In some embodiments, the mutation is a deletion, a substitution, or an insertion. In some embodiments, the mutation is a substitution. In some embodiments, the substitution is a naturally occurring amino acid. In some embodiments, an IL-12a mutein can be prepared by mutating one or more residues of IL-12a. In some embodiments, an IL-12b mutein can be prepared by mutating one or more residues of IL-12b. In some embodiments, the IL-12 mutein molecule comprises a mutation in the polypeptide sequence at a position of amino acid 220. In some embodiments, the IL-12 mutein molecule comprises a mutation in the polypeptide sequence at a position of amino acid 222. In some embodiments, the IL-12 mutein molecule comprises a mutation in the polypeptide sequence at a position of amino acid 220 and/or 222. In some embodiments, the IL-12 comprises a N222L mutation. In some embodiments, the IL-12 comprises a N222L mutation in the IL-12b amino acid sequence. In some embodiments, the IL-12 comprises a N222L mutation in the IL-12b amino acid sequence as compared to SEQ ID NO: 10. In some embodiments, the IL-12b mutein comprises a N222L mutation that corresponds to SEQ ID NO: 10. In some embodiments, the IL-12 comprises a N222Q mutation. In some embodiments, the IL-12 comprises a N222Q mutation in the IL-12b amino acid sequence. In some embodiments, the IL-12 comprises a N222Q mutation in the IL-12b amino acid sequence as compared to SEQ ID NO: 10. In some embodiments, the IL-12b mutein comprises a N222Q mutation that corresponds to SEQ ID NO: 10. In some embodiments, the IL-12 comprises a N220L mutation. In some embodiments, the IL-12 comprises a N220L mutation in the IL-12b amino acid sequence. In some embodiments, the IL-12 comprises a N220L mutation in the IL-12b amino acid sequence as compared to SEQ ID NO: 11. In some embodiments, the IL-12b mutein comprises a N220L mutation that corresponds to SEQ ID NO: 11. In some embodiments, an IL-12b mutein can be prepared by mutating one or more of the residues of IL-12b. In some embodiments, the IL-12a comprises a mutation at any position as compared to SEQ ID NO: 9. In some embodiments, the IL-12b comprises a mutation at any position as compared to SEQ ID NO: 10. In some embodiments, the IL-12b comprises a mutation at any position as compared to SEQ ID NO: 11. In some embodiments, the IL-12b mutein comprises a mutation of asparagine at position 222 as compared to SEQ ID NO: 10. In some embodiments, the IL-12b mutein comprises a substitution at position N222 as compared to SEQ ID NO: 10. In some embodiments, the IL-12b mutein comprises a mutation of N222L as compared to SEQ ID NO: 10. In some embodiments, the IL-12b mutein comprises a mutation of N222Q as compared to SEQ ID NO: 10. In some embodiments, the IL-12b mutein comprises a mouse IL-12b as set forth in SEQ ID NO: 11. In some embodiments, the mouse IL-12b mutein comprises a mutation of N220L as compared to SEQ ID NO: 11.

(SEQ ID NO: 11)
MCPQKLTISWFAIVLLVSPLMAMWELEKDVYVVEVDWTPDAPGETVNLTC
DTPEEDDITWTSDQRHGVIGSGKTLTITVKEFLDAGQYTCHKGGETLSHS
HLLLHKKENGIWSTEILKNFKNKTFLKCEAPNYSGRFTCSWLVQRNMDLK
FNIKSSSSSPDSRAVTCGMASLSAEKVTLDQRDYEKYSVSCQEDVTCPTA
EETLPIELALEARQQNKYENYSTSFFIRDIIKPDPPKNLQMKPLKNSQVE
VSWEYPDSWSTPHSYFSLKFFVRIQRKKEKMKETEEGCNQKGAFLVEKTS
TEVQCKGGNVCVQAQDRYYNSSCSKWACVPCRVRS
mouse IL-12b.

In some embodiments, the IL-12 mutein, the IL-12a mutein, and the IL-12b mutein are as provided in PCT Publication Nos. WO2001068802, and WO2017201350, each of which is incorporated hereby by reference in its entirety.

In some embodiments, the protein of interest may be an antibody. An antibody comprises four polypeptides, two heavy chains and two light chains, which join to form a “Y” shaped molecule. Antibodies are classified according to the type of heavy chains they comprise. In some embodiments, engineering a cell to produce or secrete an antibody consists of a base epithelial cell line and a vector system. In some embodiments, the vector system may be a highly modular PiggyBAC transposon system. In some embodiments, the vector system comprises a strong promoter sequence that is constitutively expressed, a Kozak translation initiation sequence, a signaling peptide, the sequence for the heavy chain (HC) variable region for the antibody of interest, the sequence for the HC constant region for the antibody of interest, a poly-A tail, a second strong promoter that is constitutively expressed, a second Kozak translation initiation sequence, a second signaling peptide, the sequence for the light chain (LC) variable region for the antibody of interest, the sequence for the LC constant region for the antibody of interest, a poly-A tail, and a puromycin resistance gene between two inverted terminal repeat sequences.

In some embodiments, the antibody or antibody fragment may be an immune checkpoint inhibitor. Immune checkpoint proteins either amplify a signal (e.g., co-stimulatory molecules) or quiet a signal. Modulating the activity of immune checkpoint proteins, such as via immune checkpoint inhibitors, may allow for nuanced control over an immune response. Immune checkpoint proteins that may be targeted by immune checkpoint blockade include: adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCDILG2, PSMB 10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). Immune checkpoint inhibitors may target the PD-1 axis and/or CTLA-4.

In some embodiments, the immune checkpoint inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody. In some embodiments, the immune checkpoint inhibitor is an anti-PD-1 antibody. In some embodiments, the immune checkpoint inhibitor is an anti-PD-L1 antibody. In some embodiments, the immune checkpoint inhibitor is an anti-PD-L2 antibody. In some embodiments, the immune checkpoint inhibitor is an anti-CTLA4 antibody. In some embodiments, the immune checkpoint inhibitor is an anti-TIM3 antibody. In some embodiments, the immune checkpoint inhibitor is an anti-LAG3 antibody. In some embodiments, the immune checkpoint inhibitor is an anti-VISTA antibody. In some embodiments, the immune checkpoint inhibitor is an anti-BTLA antibody. In some embodiments, the immune checkpoint inhibitor is an anti-TIGIT antibody. In some embodiments, the immune checkpoint inhibitor is an anti-LAIR1 antibody. In some embodiments, the immune checkpoint inhibitor is an anti-CD73 antibody. In some embodiments, the immune checkpoint inhibitor is an anti-CD160 antibody. In some embodiments, the immune checkpoint inhibitor is an anti-2B4 antibody. In some embodiments, the immune checkpoint inhibitor is an anti-TGFRβ antibody.

In some embodiments, the immune checkpoint inhibitor is an anti-CTLA4 antibody. CTLA4 (see, e.g., GenBank Accession No. L15006) is found on the surface of T-cells and binds to CD80 or CD86 molecules present on the surface of antigen-presenting cells. When bound, CTLA4 transmits an inhibitory signal to the T-cell. In some embodiments, the anti-CTLA4 antibody may be selected from the group consisting of tremelimumab (formerly ticilimumab) or ipilimumab. In some embodiments, the anti-CTLA4 antibody is encoded by the aCTLA4_CAG_EF1a_CMV_hIgK-CL (human) plasmid comprising the nucleic acid sequence of:

(SEQ ID NO: 18)
TTAACCCTAGAAAGATAGTCTGCGTAAAATTGACGCATGCATTCTTGAAA
TATTGCTCTCTCTTTCTAAATAGCGCGAATCCGTCGCTGTGCATTTAGGA
CATCTCAGTCGCCGCTTGGAGCTCCCGTGAGGCGTGCTTGTCAATGCGGT
AAGTGTCACTGATTTTGAACTATAACGACCGCGTGAGTCAAAATGACGCA
TGATTATCTTTTACGTGACTTTTAAGATTTAACTCATACGATAATTATAT
TGTTATTTCATGTTCTACTTACGTGATAACTTATTATATATATATTTTCT
TGTTATAGATATCATCAACTTTGTATAGAAAAGTTGCTCGACATTGATTA
TTGACTAGTTATTAATAGTAATCAATTACGGGGTCATTAGTTCATAGCCC
ATATATGGAGTTCCGCGTTACATAACTTACGGTAAATGGCCCGCCTGGCT
GACCGCCCAACGACCCCCGCCCATTGACGTCAATAATGACGTATGTTCCC
ATAGTAACGCCAATAGGGACTTTCCATTGACGTCAATGGGTGGAGTATTT
ACGGTAAACTGCCCACTTGGCAGTACATCAAGTGTATCATATGCCAAGTA
CGCCCCCTATTGACGTCAATGACGGTAAATGGCCCGCCTGGCATTATGCC
CAGTACATGACCTTATGGGACTTTCCTACTTGGCAGTACATCTACGTATT
AGTCATCGCTATTACCATGGTCGAGGTGAGCCCCACGTTCTGCTTCACTC
TCCCCATCTCCCCCCCCTCCCCACCCCCAATTTTGTATTTATTTATTTTT
TAATTATTTTGTGCAGCGATGGGGGGGGGGGGGGGGGGGGGGGGCGCGCC
AGGCGGGGCGGGGCGGGGCGAGGGGCGGGGCGGGGCGAGGCGGAGAGGTG
CGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGG
AGTCGCTGCGCGCTGCCTTCGCCCCGTGCCCCGCTCCGCCGCCGCCTCGC
GCCGCCCGCCCCGGCTCTGACTGACCGCGTTACTCCCACAGGTGAGCGGG
CGGGACGGCCCTTCTCCTCCGGGCTGTAATTAGCGCTTGGTTTAATGACG
GCTTGTTTCTTTTCTGTGGCTGCGTGAAAGCCTTGAGGGGCTCCGGGAGG
GCCCTTTGTGCGGGGGGAGCGGCTCGGGGGGTGCGTGCGTGTGTGTGTGC
GTGGGGAGCGCCGCGTGCGGCTCCGCGCTGCCCGGCGGCTGTGAGCGCTG
CGGGCGCGGCGCGGGGCTTTGTGCGCTCCGCAGTGTGCGCGAGGGGAGCG
CGGCCGGGGGCGGTGCCCCGCGGTGCGGGGGGGGCTGCGAGGGGAACAAA
GGCTGCGTGCGGGGTGTGTGCGTGGGGGGGTGAGCAGGGGGTGTGGGCGC
GTCGGTCGGGCTGCAACCCCCCCTGCACCCCCCTCCCCGAGTTGCTGAGC
ACGGCCCGGCTTCGGGTGCGGGGCTCCGTACGGGGCGTGGCGCGGGGCTC
GCCGTGCCGGGCGGGGGGTGGCGGCAGGTGGGGGTGCCGGGCGGGGCGGG
GCCGCCTCGGGCCGGGGAGGGCTCGGGGGAGGGGCGCGGCGGCCCCCGGA
GCGCCGGCGGCTGTCGAGGCGCGGCGAGCCGCAGCCATTGCCTTTTATGG
TAATCGTGCGAGAGGGCGCAGGGACTTCCTTTGTCCCAAATCTGTGCGGA
GCCGAAATCTGGGAGGCGCCGCCGCACCCCCTCTAGCGGGCGCGGGGCGA
AGCGGTGCGGCGCCGGCAGGAAGGAAATGGGCGGGGAGGGCCTTCGTGCG
TCGCCGCGCCGCCGTCCCCTTCTCCCTCTCCAGCCTCGGGGCTGTCCGCG
GGGGGACGGCTGCCTTCGGGGGGGACGGGGCAGGGCGGGGTTCGGCTTCT
GGCGTGTGACCGGCGGCTCTAGAGCCTCTGCTAACCATGTTCATGCCTTC
TTCTTTTTCCTACAGCTCCTGGGCAACGTGCTGGTTATTGTGCTGTCTCA
TCATTTTGGCAAAGAATTGCAAGTTTGTACAAAAAAGCAGGCTGCCACCA
TGTACAGGATGCAACTCCTGTCTTGCATTGCACTAAGTCTTGCACTTGTC
ACGAATTCGCAGGTGCAGCTGGTGGAAAGCGGCGGCGGCGTGGTGCAGCC
GGGCCGCAGCCTGCGCCTGAGCTGCGCGGCGAGCGGCTTTACCTTTAGCA
GCTATACCATGCATTGGGTGCGCCAGGCGCCGGGCAAAGGCCTGGAATGG
GTGACCTTTATTAGCTATGATGGCAACAACAAATATTATGCGGATAGCGT
GAAAGGCCGCTTTACCATTAGCCGCGATAACAGCAAAAACACCCTGTATC
TGCAGATGAACAGCCTGCGCGCGGAAGATACCGCGATTTATTATTGCGCG
CGCACCGGCTGGCTGGGCCCGTTTGATTATTGGGGCCAGGGCACCCTGGT
GACCGTGAGCAGCGCGAGCACCAAAGGCCCGAGCGTGTTTCCGCTGGCGC
CGAGCAGCAAAAGCACCAGCGGCGGCACCGCGGCGCTGGGCTGCCTGGTG
AAAGATTATTTTCCGGAACCGGTGACCGTGAGCTGGAACAGCGGCGCGCT
GACCAGCGGCGTGCATACCTTTCCGGCGGTGCTGCAGAGCAGCGGCCTGT
ATAGCCTGAGCAGCGTGGTGACCGTGCCGAGCAGCAGCCTGGGCACCCAG
ACCTATATTTGCAACGTGAACCATAAACCGAGCAACACCAAAGTGGATAA
ACGCGTGGAACCGAAAAGCTGCGATAAAACCCATACCTGCCCGCCGTGCC
CGGCGCCGGAACTGCTGGGCGGCCCGAGCGTGTTTCTGTTTCCGCCGAAA
CCGAAAGATACCCTGATGATTAGCCGCACCCCGGAAGTGACCTGCGTGGT
GGTGGATGTGAGCCATGAAGATCCGGAAGTGAAATTTAACTGGTATGTGG
ATGGCGTGGAAGTGCATAACGCGAAAACCAAACCGCGCGAAGAACAGTAT
AACAGCACCTATCGCGTGGTGAGCGTGCTGACCGTGCTGCATCAGGATTG
GCTGAACGGCAAAGAATATAAATGCAAAGTGAGCAACAAAGCGCTGCCGG
CGCCGATTGAAAAAACCATTAGCAAAGCGAAAGGCCAGCCGCGCGAACCG
CAGGTGTATACCCTGCCGCCGAGCCGCGATGAACTGACCAAAAACCAGGT
GAGCCTGACCTGCCTGGTGAAAGGCTTTTATCCGAGCGATATTGCGGTGG
AATGGGAAAGCAACGGCCAGCCGGAAAACAACTATAAAACCACCCCGCCG
GTGCTGGATAGCGATGGCAGCTTTTTTCTGTATAGCAAACTGACCGTGGA
TAAAAGCCGCTGGCAGCAGGGCAACGTGTTTAGCTGCAGCGTGATGCATG
AAGCGCTGCATAACCATTATACCCAGAAAAGCCTGAGCCTGAGCCCGGGC
AAAGCTAGCACCAAGGGCCCATCGGTCTTCCCCCTGGCACCCTCCTCCAA
GAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAG
CAGCGTGGTGACCGTGCCCTCCAGCAGCTTGGGCACCCAGACCTACATCT
GCAACGTGAATCACAAGCCCAGCAACACCAAGGTGGACAAGAAAGTTGAG
CCCAAATCTTGTGACAAAACTCACACATGCCCACCGTGCCCAGCACCTGA
ACTCCTGGGGGGACCGTCAGTCTTCCTCTTCCCCCCAAAACCCAAGGACA
CCCTCATGATCTCCCGGACCCCTGAGGTCACATGCGTGGTGGTGGACGTG
AGCCACGAAGACCCTGAGGTCAAGTTCAACTGGTACGTGGACGGCGTGGA
GGTGCATAATGCCAAGACAAAGCCGCGGGAGGAGCAGTACAACAGCACGT
ACCGTGTGGTCAGCGTCCTCACCGTCCTGCACCAGGACTGGCTGAATGGC
AAGGAGTACAAGTGCAAGGTCTCCAACAAAGCCCTCCCAGCCCCCATCGA
GAAAACCATCTCCAAAGCCAAAGGGCAGCCCCGAGAACCACAGGTGTACA
CCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAG
CAATGGGCAGCCGGAGAACAACTACAAGACCACGCCTCCCGTGCTGGACT
CCGACGGCTCCTTCTTCCTCTACAGCAAGCTCACCGTGGACAAGAGCAGG
TGGCAGCAGGGGAACGTCTTCTCATGCTCCGTGATGCATGAGGCTCTGCA
CAACCACTACACGCAGAAGAGCCTCTCCCTGTCTCCGGGTAAATAGACCC
AGCTTTCTTGTACAAAGTGGCTGTGCCTTCTAGTTGCCAGCCATCTGTTG
TTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATT
GGGAAGACAATAGCAGGCATGCTGGGGATGCGGTGGGCTCTATGGGGCTC
CGGTGCCCGTCAGTGGGCAGAGCGCACATCGCCCACAGTCCCCGAGAAGT
TGGGGGGAGGGGTCGGCAATTGAACCGGTGCCTAGAGAAGGTGGCGCGGG
GTAAACTGGGAAAGTGATGTCGTGTACTGGCTCCGCCTTTTTCCCGAGGG
TGGGGGAGAACCGTATATAAGTGCAGTAGTCGCCGTGAACGTTCTTTTTC
GCAACGGGTTTGCCGCCAGAACACAGGTAAGTGCCGTGTGTGGTTCCCGC
GGGCCTGGCCTCTTTACGGGTTATGGCCCTTGCGTGCCTTGAATTACTTC
CACCTGGCTGCAGTACGTGATTCTTGATCCCGAGCTTCGGGTTGGAAGTG
GGTGGGAGAGTTCGAGGCCTTGCGCTTAAGGAGCCCCTTCGCCTCGTGCT
TGAGTTGAGGCCTGGCCTGGGCGCTGGGGCCGCCGCGTGCGAATCTGGTG
GCACCTTCGCGCCTGTCTCGCTGCTTTCGATAAGTCTCTAGCCATTTAAA
ATTTTTGATGACCTGCTGCGACGCTTTTTTTCTGGCAAGATAGTCTTGTA
AATGCGGGCCAAGATCTGCACACTGGTATTTCGGTTTTTGGGGCCGCGGG
CGGCGACGGGGCCCGTGCGTCCCAGCGCACATGTTCGGCGAGGCGGGGCC
TGCGAGCGCGGCCACCGAGAATCGGACGGGGGTAGTCTCAAGCTGGCCGG
CCTGCTCTGGTGCCTGGTCTCGCGCCGCCGTGTATCGCCCCGCCCTGGGC
GGCAAGGCTGGCCCGGTCGGCACCAGTTGCGTGAGCGGAAAGATGGCCGC
TTCCCGGCCCTGCTGCAGGGAGCTCAAAATGGAGGACGCGGCGCTCGGGA
GAGCGGGCGGGTGAGTCACCCACACAAAGGAAAAGGGCCTTTCCGTCCTC
AGCCGTCGCTTCATGTGACTCCACGGAGTACCGGGCGCCGTCCAGGCACC
TCGATTAGTTCTCGAGCTTTTGGAGTACGTCGTCTTTAGGTTGGGGGGAG
GGGTTTTATGCGATGGAGTTTCCCCACACTGAGTGGGTGGAGACTGAAGT
TAGGCCAGCTTGGCACTTGATGTAATTCTCCTTGGAATTTGCCCTTTTTG
AGTTTGGATCTTGGTTCATTCTCAAGCCTCAGACAGTGGTTCAAAGTTTT
TTTCTTCCATTTCAGGTGTCGTGAGCCACCATGTACAGGATGCAACTCCT
GTCTTGCATTGCACTAAGTCTTGCACTTGTCACGAATTCGGAAATTGTGC
TGACCCAGAGCCCGGGCACCCTGAGCCTGAGCCCGGGCGAACGCGCGACC
CTGAGCTGCCGCGCGAGCCAGAGCGTGGGCAGCAGCTATCTGGCGTGGTA
TCAGCAGAAACCGGGCCAGGCGCCGCGCCTGCTGATTTATGGCGCGTTTA
GCCGCGCGACCGGCATTCCGGATCGCTTTAGCGGCAGCGGCAGCGGCACC
GATTTTACCCTGACCATTAGCCGCCTGGAACCGGAAGATTTTGCGGTGTA
TTATTGCCAGCAGTATGGCAGCAGCCCGTGGACCTTTGGCCAGGGCACCA
AAGTGGAAATTAAACGCACCGTGGCGGCGCCGAGCGTGTTTATTTTTCCG
CCGAGCGATGAACAGCTGAAAAGCGGCACCGCGAGCGTGGTGTGCCTGCT
GAACAACTTTTATCCGCGCGAAGCGAAAGTGCAGTGGAAAGTGGATAACG
CGCTGCAGAGCGGCAACAGCCAGGAAAGCGTGACCGAACAGGATAGCAAA
GATAGCACCTATAGCCTGAGCAGCACCCTGACCCTGAGCAAAGCGGATTA
TGAAAAACATAAAGTGTATGCGTGCGAAGTGACCCATCAGGGCCTGAGCA
GCCCGGTGACCAAAAGCTTTAACCGCGGCGAATGCACGGTGGCTGCACCA
TCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTAC
AGTGGAAGGTGGATAACGCCCTCCAATCGGGTAACTCCCAGGAGAGTGTC
ACAGAGCAGGACAGCAAGGACAGCACCTACAGCCTCAGCAGCACCCTGAC
GCTGAGCAAAGCAGACTACGAGAAACACAAAGTCTACGCCTGCGAAGTCA
CCCATCAGGGCCTGAGCTCGCCCGTCACAAAGAGCTTCAACAGGGGAGAG
TGTTAGCAACTTTATTATACATAGTTGATCCTCAGGTGCAGGCTGCCTAT
CAGAAGGTGGTGGCTGGTGTGGCCAATGCCCTGGCTCACAAATACCACTG
AGATCTTTTTCCCTCTGCCAAAAATTATGGGGACATCATGAAGCCCCTTG
AGCATCTGACTTCTGGCTAATAAAGGAAATTTATTTTCATTGCAATAGTG
TGTTGGAATTTTTTGTGTCTCTCACTCGGAAGGACATATGGGAGGGCAAA
TCATTTAAAACATCAGAATGAGTATTTGGTTTAGAGTTTGGCAACATATG
CCCATATGCTGGCTGCCATGAACAAAGGTTGGCTATAAAGAGGTCATCAG
TATATGAAACAGCCCCCTGCTGTCCATTCCTTATTCCATAGAAAAGCCTT
GACTTGAGGTTAGATTTTTTTTATATTTTGTTTTGTGTTATTTTTTTCTT
TAACATCCCTAAAATTTTCCTTACATGTTTTACTAGCCAGATTTTTCCTC
CTCTCCTGACTACTCCCAGTCATAGCTGTCCCTCTTCTCTTATGGAGATC
CCTCGACCTGCAGCCCAAGCTTCGCGTTGACATTGATTATTGACTAGTTA
TTAATAGTAATCAATTACGGGGTCATTAGTTCATAGCCCATATATGGAGT
TCCGCGTTACATAACTTACGGTAAATGGCCCGCCTGGCTGACCGCCCAAC
GACCCCCGCCCATTGACGTCAATAATGACGTATGTTCCCATAGTAACGCC
AATAGGGACTTTCCATTGACGTCAATGGGTGGAGTATTTACGGTAAACTG
CCCACTTGGCAGTACATCAAGTGTATCATATGCCAAGTACGCCCCCTATT
GACGTCAATGACGGTAAATGGCCCGCCTGGCATTATGCCCAGTACATGAC
CTTATGGGACTTTCCTACTTGGCAGTACATCTACGTATTAGTCATCGCTA
TTACCATGGTGATGCGGTTTTGGCAGTACATCAATGGGCGTGGATAGCGG
TTTGACTCACGGGGATTTCCAAGTCTCCACCCCATTGACGTCAATGGGAG
TTTGTTTTGGCACCAAAATCAACGGGACTTTCCAAAATGTCGTAACAACT
CCGCCCCATTGACGCAAATGGGCGGTAGGCGTGTACGGTGGGAGGTCTAT
ATAAGCAGAGCTCTCTGGCTAACTAGAGAACCCACTGCGCCACCATGACC
GAGTACAAGCCCACGGTGCGCCTCGCCACCCGCGACGACGTCCCCAGGGC
CGTACGCACCCTCGCCGCCGCGTTCGCCGACTACCCCGCCACGCGCCACA
CCGTCGATCCGGACCGCCACATCGAGCGGGTCACCGAGCTGCAAGAACTC
TTCCTCACGCGCGTCGGGCTCGACATCGGCAAGGTGTGGGTCGCGGACGA
CGGCGCCGCGGTGGCGGTCTGGACCACGCCGGAGAGCGTCGAAGCGGGGG
CGGTGTTCGCCGAGATCGGCCCGCGCATGGCCGAGTTGAGCGGTTCCCGG
CTGGCCGCGCAGCAACAGATGGAAGGCCTCCTGGCGCCGCACCGGCCCAA
GGAGCCCGCGTGGTTCCTGGCCACCGTCGGCGTCTCGCCCGACCACCAGG
GCAAGGGTCTGGGCAGCGCCGTCGTGCTCCCCGGAGTGGAGGCGGCCGAG
CGCGCCGGGGTGCCCGCCTTCCTGGAGACCTCCGCGCCCCGCAACCTCCC
CTTCTACGAGCGGCTCGGCTTCACCGTCACCGCCGACGTCGAGGTGCCCG
AAGGACCGCGCACCTGGTGCATGACCCGCAAGCCCGGTGCCTGACTCGAG
TCTAGAGGGCCCGTTTAAACCCGCTGATCAGCCTCGACTGTGCCTTCTAG
TTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGG
TGGGCTCTATGGCTCGAGTTAATTAACGAGAGCATAATATTGATATGTGC
CAAAGTIGTTTCTGACTGACTAATAAGTATAATTTGTTTCTATTATGTAT
AGGTTAAGCTAATTACTTATTTTATAATACAACATGACTGTTTTTAAAGT
ACAAAATAAGTTTATTTTTGTAAAAGAGAGAATGTTTAAAAGTTTTGTTA
CTTTATAGAAGAAATTTTGAGTTTTTGTTTTTTTTTAATAAATAAATAAA
CATAAATAAATTGTTTGTTGAATTTATTATTAGTATGTAAGTGTAAATAT
AATAAAACTTAATATCTATTCAAATTAATAAATAAACCTCGATATACAGA
CCGATAAAACACATGCGTCAATTTTACGCATGATTATCTTTAACGTACGT
CACAATATGATTATCTTTCTAGGGTTAAATAATAGTTTCTAATTTTTTTA
TTATTCAGCCTGCTGTCGTGAATACCGAGCTCCAATTCGCCCTATAGTGA
GTCGTATTACAATTCACTGGCCGTCGTTTTACAACGTCGTGACTGGGAAA
ACCCTGGCGTTACCCAACTTAATCGCCTTGCAGCACATCCCCCTTTCGCC
AGCTGGCGTAATAGCGAAGAGGCCCGCACCGATCGCCCTTCCCAACAGTT
GCGCAGCCTGAATGGCGAATGGGACGCGCCCTGTAGCGGCGCATTAAGCG
CGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGAT
TTAGTGCTTTACGGCACCTCGACCCCAAAAAACTTGATTAGGGTGATGGT
TCACGTAGTGGGCCATCGCCCTGATAGACGGTTTTTCGCCCTTTGACGTT
GGAGTCCACGTTCTTTAATAGTGGACTCTTGTTCCAAACTGGAACAACAC
TCAACCCTATCTCGGTCTATTCTTTTGATTTATAAGGGATTTTGCCGATT
TCGGCCTATTGGTTAAAAAATGAGCTGATTTAACAAAAATTTAACGCGAA
TTTTAACAAAATATTAACGCTTACAATTTAGGTGGCACTTTTCGGGGAAA
TGTGCGCGGAACCCCTATTTGTTTATTTTTCTAAATACATTCAAATATGT
ATCCGCTCATGAGACAATAACCCTGATAAATGCTTCAATAATATTGAAAA
AGGAAGAGTATGAGTATTCAACATTTCCGTGTCGCCCTTATTCCCTTTTT
TGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTG
GATCTCAACAGCGGTAAGATCCTTGAGAGTTTTCGCCCCGAAGAACGTTT
TCCAATGATGAGCACTTTTAAAGTTCTGCTATGTGGCGCGGTATTATCCC
GTATTGACGCCGGGCAAGAGCAACTCGGTCGCCGCATACACTATTCTCAG
AATGACTTGGTTGAGTACTCACCAGTCACAGAAAAGCATCTTACGGATGG
CATGACAGTAAGAGAATTATGCAGTGCTGCCATAACCATGAGTGATAACA
CTGCGGCCAACTTACTTCTGACAACGATCGGAGGACCGAAGGAGCTAACC
GCTTTTTTGCACAACATGGGGGATCATGTAACTCGCCTTGATCGTTGGGA
ACCGGAGCTGAATGAAGCCATACCAAACGACGAGCGTGACACCACGATGC
CTGTAGCAATGGCAACAACGTTGCGCAAACTATTAACTGGCGAACTACTT
ACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTG
ATAAATCTGGAGCCGGTGAGCGTGGGTCTCGCGGTATCATTGCAGCACTG
GGGCCAGATGGTAAGCCCTCCCGTATCGTAGTTATCTACACGACGGGGAG
TCAGGCAACTATGGATGAACGAAATAGACAGATCGCTGAGATAGGTGCCT
CACTGATTAAGCATTGGTAACTGTCAGACCAAGTTTACTCATATATACTT
TAGATTGATTTAAAACTTCATTTTTAATTTAAAAGGATCTAGGTGAAGAT
CCTTTTTGATAATCTCATGACCAAAATCCCTTAACGTGAGTTTTCGTTCC
ACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACC
AGCGGTGGTTTGTTTGCCGGATCAAGAGCTACCAACTCTTTTTCCGAAGG
TAACTGGCTTCAGCAGAGCGCAGATACCAAATACTGTTCTTCTAGTGTAG
CCGTAGTTAGGCCACCACTTCAAGAACTCTGTAGCACCGCCTACATACCT
CGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGG
TCGGGCTGAACGGGGGGTTCGTGCACACAGCCCAGCTTGGAGCGAACGAC
CTACACCGAACTGAGATACCTACAGCGTGAGCTATGAGAAAGCGCCACGC
TTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTA
TAGTCCTGTCGGGTTTCGCCACCTCTGACTTGAGCGTCGATTTTTGTGAT
GCTCGTCAGGGGGGCGGAGCCTATGGAAAAACGCCAGCAACGCGGCCTTT
TTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTG
ATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCC
GATTCATTAATGCAGCTGGCACGACAGGTTTCCCGACTGGAAAGCGGGCA
GTGAGCGCAACGCAATTAATGTGAGTTAGCTCACTCATTAGGCACCCCAG
GCTTTACACTTTATGCTTCCGGCTCGTATGTTGTGTGGAATTGTGAGCGG
ATAACAATTTCACACAGGAAACAGCTATGACCATGATTACGCCAAGCTCG
AAATTAACCCTCACTAAAGGGAACAAAAGCTGGTACCTCGCGCGACTTGG
TTTGCCATTCTTTAGCGCGCGTCGCGTCACACAGCTTGGCCACAATGTGG
TTTTTGTCAAACGAAGATTCTATGACGTGTTTAAAGTTTAGGTCGAGTAA
AGCGCAAATCTTTT

In some embodiments, the anti-CTLA4 antibody is encoded by the aCTLA4 CAG_EF1a_CMV_hIgy2-CL (human) plasmid comprising the nucleic acid sequence of:

(SEQ ID NO: 19)
TTAACCCTAGAAAGATAGTCTGCGTAAAATTGACGCATGCATTCTTGAAAT
ATTGCTCTCTCTTTCTAAATAGCGCGAATCCGTCGCTGTGCATTTAGGAC
ATCTCAGTCGCCGCTTGGAGCTCCCGTGAGGCGTGCTTGTCAATGCGGTA
AGTGTCACTGATTTTGAACTATAACGACCGCGTGAGTCAAAATGACGCAT
GATTATCTTTTACGTGACTTTTAAGATTTAACTCATACGATAATTATATT
GTTATTTCATGTTCTACTTACGTGATAACTTATTATATATATATTTTCTT
GTTATAGATATCATCAACTTTGTATAGAAAAGTTGCTCGACATTGATTAT
TGACTAGTTATTAATAGTAATCAATTACGGGGTCATTAGTTCATAGCCCA
TATATGGAGTTCCGCGTTACATAACTTACGGTAAATGGCCCGCCTGGCTG
ACCGCCCAACGACCCCCGCCCATTGACGTCAATAATGACGTATGTTCCCA
TAGTAACGCCAATAGGGACTTTCCATTGACGTCAATGGGTGGAGTATTTA
CGGTAAACTGCCCACTTGGCAGTACATCAAGTGTATCATATGCCAAGTAC
GCCCCCTATTGACGTCAATGACGGTAAATGGCCCGCCTGGCATTATGCCC
AGTACATGACCTTATGGGACTTTCCTACTTGGCAGTACATCTACGTATTA
GTCATCGCTATTACCATGGTCGAGGTGAGCCCCACGTTCTGCTTCACTCT
CCCCATCTCCCCCCCCTCCCCACCCCCAATTTTGTATTTATTTATTTTTT
AATTATTTTGTGCAGCGATGGGGGGGGGGGGGGGGGGGGGGGGCGCGCCA
GGCGGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGGCGA
GGCGGCGGCGGCGGCGGCCCTATAAAAAGCGAAGCGCGCGGCGGGCGGGA
GTCGCTGCGCGCTGCCTTCGCCCCGTGCCCCGCTCCGCCGCCGCCTCGCG
CCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGG
CTTGTTTCTTTTCTGTGGCTGCGTGAAAGCCTTGAGGGGCTCCGGGAGGG
CCCTTTGTGCGGGGGGAGCGGCTCGGGGGGTGCGTGCGTGTGTGTGTGCG
TGGGGAGCGCCGCGTGCGGCTCCGCGCTGCCCGGCGGCTGTGAGCGCTGC
GGGCGCGGCGCGGGGCTTTGTGCGCTCCGCAGTGTGCGCGAGGGGAGCGC
GGCCGGGGGCGGTGCCCCGCGGTGCGGGGGGGGCTGCGAGGGGAACAAAG
GCTGCGTGCGGGGTGTGTGCGTGGGGGGGTGAGCAGGGGGTGTGGGCGCG
TCGGTCGGGCTGCAACCCCCCCTGCACCCCCCTCCCCGAGTTGCTGAGCA
CGGCCCGGCTTCGGGTGCGGGGCTCCGTACGGGGCGTGGCGCGGGGCTCG
CCGTGCCGGGCGGGGGGTGGCGGCAGGTGGGGGTGCCGGGCGGGGCGGGG
CCGCCTCGGGCCGGGGAGGGCTCGGGGGAGGGGCGCGGCGGCCCCCGGAG
CGCCGGCGGCTGTCGAGGCGCGGCGAGCCGCAGCCATTGCCTTTTATGGT
AATCGTGCGAGAGGGCGCAGGGACTTCCTTTGTCCCAAATCTGTGCGGAG
CCGAAATCTGGGAGGCGCCGCCGCACCCCCTCTAGCGGGCGCGGGGCGAA
GCGGTGCGGCGCCGGCAGGAAGGAAATGGGCGGGGAGGGCCTTCGTGCGT
CGCCGCGCCGCCGTCCCCTTCTCCCTCTCCAGCCTCGGGGCTGTCCGCGG
GGGGACGGCTGCCTTCGGGGGGGACGGGGCAGGGCGGGGTTCGGCTTCTG
GCGTGTGACCGGCGGCTCTAGAGCCTCTGCTAACCATGTTCATGCCTTCT
TCTTTTTCCTACAGCTCCTGGGCAACGTGCTGGTTATTGTGCTGTCTCAT
CATTTTGGCAAAGAATTGCAAGTTTGTACAAAAAAGCAGGCTGCCACCAT
GTACAGGATGCAACTCCTGTCTTGCATTGCACTAAGTCTTGCACTTGTCA
CGAATTCGCAGGTGCAGCTGGTGGAAAGCGGCGGCGGCGTGGTGCAGCCG
GGCCGCAGCCTGCGCCTGAGCTGCGCGGCGAGCGGCTTTACCTTTAGCAG
CTATACCATGCATTGGGTGCGCCAGGCGCCGGGCAAAGGCCTGGAATGGG
TGACCTTTATTAGCTATGATGGCAACAACAAATATTATGCGGATAGCGTG
AAAGGCCGCTTTACCATTAGCCGCGATAACAGCAAAAACACCCTGTATCT
GCAGATGAACAGCCTGCGCGCGGAAGATACCGCGATTTATTATTGCGCGC
GCACCGGCTGGCTGGGCCCGTTTGATTATTGGGGCCAGGGCACCCTGGTG
ACCGTGAGCAGCGCGAGCACCAAAGGCCCGAGCGTGTTTCCGCTGGCGCC
GAGCAGCAAAAGCACCAGCGGCGGCACCGCGGCGCTGGGCTGCCTGGTGA
AAGATTATTTTCCGGAACCGGTGACCGTGAGCTGGAACAGCGGCGCGCTG
ACCAGCGGCGTGCATACCTTTCCGGCGGTGCTGCAGAGCAGCGGCCTGTA
TAGCCTGAGCAGCGTGGTGACCGTGCCGAGCAGCAGCCTGGGCACCCAGA
CCTATATTTGCAACGTGAACCATAAACCGAGCAACACCAAAGTGGATAAA
CGCGTGGAACCGAAAAGCTGCGATAAAACCCATACCTGCCCGCCGTGCCC
GGCGCCGGAACTGCTGGGCGGCCCGAGCGTGTTTCTGTTTCCGCCGAAAC
CGAAAGATACCCTGATGATTAGCCGCACCCCGGAAGTGACCTGCGTGGTG
GTGGATGTGAGCCATGAAGATCCGGAAGTGAAATTTAACTGGTATGTGGA
TGGCGTGGAAGTGCATAACGCGAAAACCAAACCGCGCGAAGAACAGTATA
ACAGCACCTATCGCGTGGTGAGCGTGCTGACCGTGCTGCATCAGGATTGG
CTGAACGGCAAAGAATATAAATGCAAAGTGAGCAACAAAGCGCTGCCGGC
GCCGATTGAAAAAACCATTAGCAAAGCGAAAGGCCAGCCGCGCGAACCGC
AGGTGTATACCCTGCCGCCGAGCCGCGATGAACTGACCAAAAACCAGGTG
AGCCTGACCTGCCTGGTGAAAGGCTTTTATCCGAGCGATATTGCGGTGGA
ATGGGAAAGCAACGGCCAGCCGGAAAACAACTATAAAACCACCCCGCCGG
TGCTGGATAGCGATGGCAGCTTTTTTCTGTATAGCAAACTGACCGTGGAT
AAAAGCCGCTGGCAGCAGGGCAACGTGTTTAGCTGCAGCGTGATGCATGA
AGCGCTGCATAACCATTATACCCAGAAAAGCCTGAGCCTGAGCCCGGGCA
AAGCTAGCACCAAGGGCCCATCGGTCTTCCCCCTGGCACCCTCCTCCAAG
AGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCG
TGCACACCTTCCCGGCTGTCCTACAGTCCTCAGGACTCTACTCCCTCAGC
AGCGTGGTGACCGTGCCCTCCAGCAGCTTGGGCACCCAGACCTACATCTG
CAACGTGAATCACAAGCCCAGCAACACCAAGGTGGACAAGAAAGTTGAGC
CCAAATCTTGTGACAAAACTCACACATGCCCACCGTGCCCAGCACCTGAA
CTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGA
GCCACGAAGACCCTGAGGTCAAGTTCAACTGGTACGTGGACGGCGTGGAG
GTGCATAATGCCAAGACAAAGCCGCGGGAGGAGCAGTACAACAGCACGTA
CCGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACAC
CCTGCCCCCATCCCGGGAGGAGATGACCAAGAACCAGGTCAGCCTGACCT
GCCTGGTCAAAGGCTTCTATCCCAGCGACATCGCCGTGGAGTGGGAGAGC
AATGGGCAGCCGGAGAACAACTACAAGACCACGCCTCCCGTGCTGGACTC
CGACGGCTCCTTCTTCCTCTACAGCAAGCTCACCGTGGACAAGAGCAGGT
GGCAGCAGGGGAACGTCTTCTCATGCTCCGTGATGCATGAGGCTCTGCAC
AACCACTACACGCAGAAGAGCCTCTCCCTGTCTCCGGGTAAATAGACCCA
GCTTTCTTGTACAAAGTGGCTGTGCCTTCTAGTTGCCAGCCATCTGTTGT
TTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTG
GGAAGACAATAGCAGGCATGCTGGGGATGCGGTGGGCTCTATGGGGCTCC
GGTGCCCGTCAGTGGGCAGAGCGCACATCGCCCACAGTCCCCGAGAAGTT
GGGGGGAGGGGTCGGCAATTGAACCGGTGCCTAGAGAAGGTGGCGCGGGG
TAAACTGGGAAAGTGATGTCGTGTACTGGCTCCGCCTTTTTCCCGAGGGT
GGGGGAGAACCGTATATAAGTGCAGTAGTCGCCGTGAACGTTCTTTTTCG
CAACGGGTTTGCCGCCAGAACACAGGTAAGTGCCGTGTGTGGTTCCCGCG
GGCCTGGCCTCTTTACGGGTTATGGCCCTTGCGTGCCTTGAATTACTTCC
ACCTGGCTGCAGTACGTGATTCTTGATCCCGAGCTTCGGGTTGGAAGTGG
GTGGGAGAGTTCGAGGCCTTGCGCTTAAGGAGCCCCTTCGCCTCGTGCTT
GAGTTGAGGCCTGGCCTGGGCGCTGGGGCCGCCGCGTGCGAATCTGGTGG
CACCTTCGCGCCTGTCTCGCTGCTTTCGATAAGTCTCTAGCCATTTAAAA
TTTTTGATGACCTGCTGCGACGCTTTTTTTCTGGCAAGATAGTCTTGTAA
ATGCGGGCCAAGATCTGCACACTGGTATTTCGGTTTTTGGGGCCGCGGGC
GGCGACGGGGCCCGTGCGTCCCAGCGCACATGTTCGGCGAGGCGGGGCCT
GCGAGCGCGGCCACCGAGAATCGGACGGGGGTAGTCTCAAGCTGGCCGGC
CTGCTCTGGTGCCTGGTCTCGCGCCGCCGTGTATCGCCCCGCCCTGGGCG
GCAAGGCTGGCCCGGTCGGCACCAGTTGCGTGAGCGGAAAGATGGCCGCT
TCCCGGCCCTGCTGCAGGGAGCTCAAAATGGAGGACGCGGCGCTCGGGAG
AGCGGGCGGGTGAGTCACCCACACAAAGGAAAAGGGCCTTTCCGTCCTCA
GCCGTCGCTTCATGTGACTCCACGGAGTACCGGGCGCCGTCCAGGCACCT
CGATTAGTTCTCGAGCTTTTGGAGTACGTCGTCTTTAGGTTGGGGGGAGG
GGTTTTATGCGATGGAGTTTCCCCACACTGAGTGGGTGGAGACTGAAGTT
AGGCCAGCTTGGCACTTGATGTAATTCTCCTTGGAATTTGCCCTTTTTGA
GTTTGGATCTTGGTTCATTCTCAAGCCTCAGACAGTGGTTCAAAGTTTTT
TTCTTCCATTTCAGGTGTCGTGAGCCACCATGTACAGGATGCAACTCCTG
TCTTGCATTGCACTAAGTCTTGCACTTGTCACGAATTCGGAAATTGTGCT
GACCCAGAGCCCGGGCACCCTGAGCCTGAGCCCGGGCGAACGCGCGACCC
TGAGCTGCCGCGCGAGCCAGAGCGTGGGCAGCAGCTATCTGGCGTGGTAT
CAGCAGAAACCGGGCCAGGCGCCGCGCCTGCTGATTTATGGCGCGTTTAG
CCGCGCGACCGGCATTCCGGATCGCTTTAGCGGCAGCGGCAGCGGCACCG
ATTTTACCCTGACCATTAGCCGCCTGGAACCGGAAGATTTTGCGGTGTAT
TATTGCCAGCAGTATGGCAGCAGCCCGTGGACCTTTGGCCAGGGCACCAA
AGTGGAAATTAAACGCACCGTGGCGGCGCCGAGCGTGTTTATTTTTCCGC
CGAGCGATGAACAGCTGAAAAGCGGCACCGCGAGCGTGGTGTGCCTGCTG
AACAACTTTTATCCGCGCGAAGCGAAAGTGCAGTGGAAAGTGGATAACGC
GCTGCAGAGCGGCAACAGCCAGGAAAGCGTGACCGAACAGGATAGCAAAG
ATAGCACCTATAGCCTGAGCAGCACCCTGACCCTGAGCAAAGCGGATTAT
GAAAAACATAAAGTGTATGCGTGCGAAGTGACCCATCAGGGCCTGAGCAG
CCCGGTGACCAAAAGCTTTAACCGCGGCGAATGCGGTCAGCCCAAGGCTG
CCCCCTCGGTCACTCTGTTCCCGCCCTCCTCTGAGGAGCTTCAAGCCAAC
AAGGCCACACTGGTGTGTCTCATAAGTGACTTCTACCCGGGAGCCGTGAC
AGTGGCCTGGAAGGCAGATAGCAGCCCCGTCAAGGCGGGAGTGGAGACCA
CCACACCCTCCAAACAAAGCAACAACAAGTACGCGGCCAGCAGCTATCTG
AGCCTGACGCCTGAGCAGTGGAAGTCCCACAGAAGCTACAGCTGCCAGGT
CACGCATGAAGGGAGCACCGTGGAGAAGACAGTGGCCCCTACAGAATGTT
CATAGCAACTTTATTATACATAGTTGATCCTCAGGTGCAGGCTGCCTATC
AGAAGGTGGTGGCTGGTGTGGCCAATGCCCTGGCTCACAAATACCACTGA
GATCTTTTTCCCTCTGCCAAAAATTATGGGGACATCATGAAGCCCCTTGA
GCATCTGACTTCTGGCTAATAAAGGAAATTTATTTTCATTGCAATAGTGT
GTTGGAATTTTTTGTGTCTCTCACTCGGAAGGACATATGGGAGGGCAAAT
CATTTAAAACATCAGAATGAGTATTTGGTTTAGAGTTTGGCAACATATGC
CCATATGCTGGCTGCCATGAACAAAGGTTGGCTATAAAGAGGTCATCAGT
ATATGAAACAGCCCCCTGCTGTCCATTCCTTATTCCATAGAAAAGCCTTG
ACTTGAGGTTAGATTTTTTTTATATTTTGTTTTGTGTTATTTTTTTCTTT
AACATCCCTAAAATTTTCCTTACATGTTTTACTAGCCAGATTTTTCCTCC
TCTCCTGACTACTCCCAGTCATAGCTGTCCCTCTTCTCTTATGGAGATCC
CTCGACCTGCAGCCCAAGCTTCGCGTTGACATTGATTATTGACTAGTTAT
TAATAGTAATCAATTACGGGGTCATTAGTTCATAGCCCATATATGGAGTT
CCGCGTTACATAACTTACGGTAAATGGCCCGCCTGGCTGACCGCCCAACG
ACCCCCGCCCATTGACGTCAATAATGACGTATGTTCCCATAGTAACGCCA
ATAGGGACTTTCCATTGACGTCAATGGGTGGAGTATTTACGGTAAACTGC
CCACTTGGCAGTACATCAAGTGTATCATATGCCAAGTACGCCCCCTATTG
ACGTCAATGACGGTAAATGGCCCGCCTGGCATTATGCCCAGTACATGACC
TTATGGGACTTTCCTACTTGGCAGTACATCTACGTATTAGTCATCGCTAT
TACCATGGTGATGCGGTTTTGGCAGTACATCAATGGGCGTGGATAGCGGT
TTGACTCACGGGGATTTCCAAGTCTCCACCCCATTGACGTCAATGGGAGT
TTGTTTTGGCACCAAAATCAACGGGACTTTCCAAAATGTCGTAACAACTC
CGCCCCATTGACGCAAATGGGCGGTAGGCGTGTACGGTGGGAGGTCTATA
TAAGCAGAGCTCTCTGGCTAACTAGAGAACCCACTGCGCCACCATGACCG
AGTACAAGCCCACGGTGCGCCTCGCCACCCGCGACGACGTCCCCAGGGCC
GTACGCACCCTCGCCGCCGCGTTCGCCGACTACCCCGCCACGCGCCACAC
CGTCGATCCGGACCGCCACATCGAGCGGGTCACCGAGCTGCAAGAACTCT
TCCTCACGCGCGTCGGGCTCGACATCGGCAAGGTGTGGGTCGCGGACGAC
GGCGCCGCGGTGGCGGTCTGGACCACGCCGGAGAGCGTCGAAGCGGGGGC
GGTGTTCGCCGAGATCGGCCCGCGCATGGCCGAGTTGAGCGGTTCCCGGC
TGGCCGCGCAGCAACAGATGGAAGGCCTCCTGGCGCCGCACCGGCCCAAG
GAGCCCGCGTGGTTCCTGGCCACCGTCGGCGTCTCGCCCGACCACCAGGG
CAAGGGTCTGGGCAGCGCCGTCGTGCTCCCCGGAGTGGAGGCGGCCGAGC
GCGCCGGGGTGCCCGCCTTCCTGGAGACCTCCGCGCCCCGCAACCTCCCC
TTCTACGAGCGGCTCGGCTTCACCGTCACCGCCGACGTCGAGGTGCCCGA
AGGACCGCGCACCTGGTGCATGACCCGCAAGCCCGGTGCCTGACTCGAGT
CTAGAGGGCCCGTTTAAACCCGCTGATCAGCCTCGACTGTGCCTTCTAGT
TGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGT
GGGCTCTATGGCTCGAGTTAATTAACGAGAGCATAATATTGATATGTGCC
AAAGTTGTTTCTGACTGACTAATAAGTATAATTTGTTTCTATTATGTATA
GGTTAAGCTAATTACTTATTTTATAATACAACATGACTGTTTTTAAAGTA
CAAAATAAGTTTATTTTTGTAAAAGAGAGAATGTTTAAAAGTTTTGTTAC
TTTATAGAAGAAATTTTGAGTTTTTGTTTTTTTTTAATAAATAAATAAAC
ATAAATAAATTGTTTGTTGAATTTATTATTAGTATGTAAGTGTAAATATA
ATAAAACTTAATATCTATTCAAATTAATAAATAAACCTCGATATACAGAC
CGATAAAACACATGCGTCAATTTTACGCATGATTATCTTTAACGTACGTC
ACAATATGATTATCTTTCTAGGGTTAAATAATAGTTTCTAATTTTTTTAT
TATTCAGCCTGCTGTCGTGAATACCGAGCTCCAATTCGCCCTATAGTGAG
TCGTATTACAATTCACTGGCCGTCGTTTTACAACGTCGTGACTGGGAAAA
CCCTGGCGTTACCCAACTTAATCGCCTTGCAGCACATCCCCCTTTCGCCA
GCTGGCGTAATAGCGAAGAGGCCCGCACCGATCGCCCTTCCCAACAGTTG
CGCAGCCTGAATGGCGAATGGGACGCGCCCTGTAGCGGCGCATTAAGCGC
GGCGGGTGTGGTGGTTACGCGCAGCGTGACCGCTACACTTGCCAGCGCCC
TAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATT
TAGTGCTTTACGGCACCTCGACCCCAAAAAACTTGATTAGGGTGATGGTT
CACGTAGTGGGCCATCGCCCTGATAGACGGTTTTTCGCCCTTTGACGTTG
GAGTCCACGTTCTTTAATAGTGGACTCTTGTTCCAAACTGGAACAACACT
CAACCCTATCTCGGTCTATTCTTTTGATTTATAAGGGATTTTGCCGATTT
CGGCCTATTGGTTAAAAAATGAGCTGATTTAACAAAAATTTAACGCGAAT
TTTAACAAAATATTAACGCTTACAATTTAGGTGGCACTTTTCGGGGAAAT
GTGCGCGGAACCCCTATTTGTTTATTTTTCTAAATACATTCAAATATGTA
TCCGCTCATGAGACAATAACCCTGATAAATGCTTCAATAATATTGAAAAA
GGAAGAGTATGAGTATTCAACATTTCCGTGTCGCCCTTATTCCCTTTTTT
GCGGCATTTTGCCTTCCTGTTTTTGCTCACCCAGAAACGCTGGTGAAAGT
AAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCG
TATTGACGCCGGGCAAGAGCAACTCGGTCGCCGCATACACTATTCTCAGA
ATGACTTGGTTGAGTACTCACCAGTCACAGAAAAGCATCTTACGGATGGC
ATGACAGTAAGAGAATTATGCAGTGCTGCCATAACCATGAGTGATAACAC
TGCGGCCAACTTACTTCTGACAACGATCGGAGGACCGAAGGAGCTAACCG
CTTTTTTGCACAACATGGGGGATCATGTAACTCGCCTTGATCGTTGGGAA
CCGGAGCTGAATGAAGCCATACCAAACGACGAGCGTGACACCACGATGCC
TGTAGCAATGGCAACAACGTTGCGCAAACTATTAACTGGCGAACTACTTA
CTCTAGCTTCCCGGCAACAATTAATAGACTGGATGGAGGCGGATAAAGTT
GCAGGACCACTTCTGCGCTCGGCCCTTCCGGCTGGCTGGTTTATTGCTGA
TAAATCTGGAGCCGGTGAGCGTGGGTCTCGCGGTATCATTGCAGCACTGG
GGCCAGATGGTAAGCCCTCCCGTATCGTAGTTATCTACACGACGGGGAGT
CAGGCAACTATGGATGAACGAAATAGACAGATCGCTGAGATAGGTGCCTC
ACTGATTAAGCATTGGTAACTGTCAGACCAAGTTTACTCATATATACTTT
AGATTGATTTAAAACTTCATTTTTAATTTAAAAGGATCTAGGTGAAGATC
CTTTTTGATAATCTCATGACCAAAATCCCTTAACGTGAGTTTTCGTTCCA
CTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCA
GCGGTGGTTTGTTTGCCGGATCAAGAGCTACCAACTCTTTTTCCGAAGGT
AACTGGCTTCAGCAGAGCGCAGATACCAAATACTGTTCTTCTAGTGTAGC
CGTAGTTAGGCCACCACTTCAAGAACTCTGTAGCACCGCCTACATACCTC
GCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGT
CGGGCTGAACGGGGGGTTCGTGCACACAGCCCAGCTTGGAGCGAACGACC
TACACCGAACTGAGATACCTACAGCGTGAGCTATGAGAAAGCGCCACGCT
TCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATG
CTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCG
TTATCCCCTGATTCTGTGGATAACCGTATTACCGCCTTTGAGTGAGCTGA
TACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCG
ATTCATTAATGCAGCTGGCACGACAGGTTTCCCGACTGGAAAGCGGGCAG
TGAGCGCAACGCAATTAATGTGAGTTAGCTCACTCATTAGGCACCCCAGG
CTTTACACTTTATGCTTCCGGCTCGTATGTTGTGTGGAATTGTGAGCGGA
TAACAATTTCACACAGGAAACAGCTATGACCATGATTACGCCAAGCTCGA
AATTAACCCTCACTAAAGGGAACAAAAGCTGGTACCTCGCGCGACTTGGT
TTGCCATTCTTTAGCGCGCGTCGCGTCACACAGCTTGGCCACAATGTGGT
TTTTGTCAAACGAAGATTCTATGACGTGTTTAAAGTTTAGGTCGAGTAAA
GCGCAAATCTTTT

In some embodiments, the immune checkpoint inhibitor is an anti-PD-1 antibody. PD-1 is a protein on the surface of T-cells and B-cells which bind PD-L1 and/or PD-L2. Upon binding, PD-1 transmits an inhibitory signal. In some embodiments, the anti-PD-1 antibody is selected from the group consisting of nivolumab (see, e.g., International Patent Application Pub. No. WO 2006/121168), pembrolizumab (see, e.g., International Patent Application Pub. No. WO 2009/114335), and CT-011 (see, e.g., International Patent Application Pub. No. WO 2009/101611). In some embodiments, the anti-PD-1 antibody is selected from pembrolizumab, nivolumab, cemiplimab, atezolizumab, dostralimab, durvalumab, avelumab, or any combination thereof. In some embodiments, the anti-PD-1 antibody is encoded by the aPD1_CAG_EF1a_CMV_hIgK-CL (human) plasmid comprising the nucleic acid sequence of:

(SEQ ID NO: 20)
TTAACCCTAGAAAGATAGTCTGCGTAAAATTGACGCATGCATTCTTGAAAT
ATTGCTCTCTCTTTCTAAATAGCGCGAATCCGTCGCTGTGCATTTAGGAC
ATCTCAGTCGCCGCTTGGAGCTCCCGTGAGGCGTGCTTGTCAATGCGGTA
AGTGTCACTGATTTTGAACTATAACGACCGCGTGAGTCAAAATGACGCAT
GATTATCTTTTACGTGACTTTTAAGATTTAACTCATACGATAATTATATT
GTTATTTCATGTTCTACTTACGTGATAACTTATTATATATATATTTTCTT
GTTATAGATATCATCAACTTTGTATAGAAAAGTTGCTCGACATTGATTAT
TGACTAGTTATTAATAGTAATCAATTACGGGGTCATTAGTTCATAGCCCA
TATATGGAGTTCCGCGTTACATAACTTACGGTAAATGGCCCGCCTGGCTG
ACCGCCCAACGACCCCCGCCCATTGACGTCAATAATGACGTATGTTCCCA
TAGTAACGCCAATAGGGACTTTCCATTGACGTCAATGGGTGGAGTATTTA
CGGTAAACTGCCCACTTGGCAGTACATCAAGTGTATCATATGCCAAGTAC
GCCCCCTATTGACGTCAATGACGGTAAATGGCCCGCCTGGCATTATGCCC
AGTACATGACCTTATGGGACTTTCCTACTTGGCAGTACATCTACGTATTA
GTCATCGCTATTACCATGGTCGAGGTGAGCCCCACGTTCTGCTTCACTCT
CCCCATCTCCCCCCCCTCCCCACCCCCAATTTTGTATTTATTTATTTTTT
AATTATTTTGTGCAGCGATGGGGGGGGGGGGGGGGGGGGGGGGCGCGCCA
GGCGGGGCGGGGCGGGGCGAGGGGCGGGGCGGGGCGAGGCGGAGAGGTGC
GGCGGCAGCCAATCAGAGCGGCGCGCTCCGAAAGTTTCCTTTTATGGCGA
GGCGGCGGCGGCGGCGGCCCTATAAAAAGCGAAGCGCGCGGCGGGCGGGA
GTCGCTGCGCGCTGCCTTCGCCCCGTGCCCCGCTCCGCCGCCGCCTCGCG
CCGCCCGCCCCGGCTCTGACTGACCGCGTTACTCCCACAGGTGAGCGGGC
GGGACGGCCCTTCTCCTCCGGGCTGTAATTAGCGCTTGGTTTAATGACGG
CTTGTTTCTTTTCTGTGGCTGCGTGAAAGCCTTGAGGGGCTCCGGGAGGG
CCCTTTGTGCGGGGGGAGCGGCTCGGGGGGTGCGTGCGTGTGTGTGTGCG
TGGGGAGCGCCGCGTGCGGCTCCGCGCTGCCCGGCGGCTGTGAGCGCTGC
GGGCGCGGCGCGGGGCTTTGTGCGCTCCGCAGTGTGCGCGAGGGGAGCGC
GGCCGGGGGCGGTGCCCCGCGGTGCGGGGGGGGCTGCGAGGGGAACAAAG
GCTGCGTGCGGGGTGTGTGCGTGGGGGGGTGAGCAGGGGGTGTGGGCGCG
TCGGTCGGGCTGCAACCCCCCCTGCACCCCCCTCCCCGAGTTGCTGAGCA
CGGCCCGGCTTCGGGTGCGGGGCTCCGTACGGGGCGTGGCGCGGGGCTCG
CCGTGCCGGGCGGGGGGTGGCGGCAGGTGGGGGTGCCGGGCGGGGCGGGG
CCGCCTCGGGCCGGGGAGGGCTCGGGGGAGGGGCGCGGCGGCCCCCGGAG
CGCCGGCGGCTGTCGAGGCGCGGCGAGCCGCAGCCATTGCCTTTTATGGT
AATCGTGCGAGAGGGCGCAGGGACTTCCTTTGTCCCAAATCTGTGCGGAG
CCGAAATCTGGGAGGCGCCGCCGCACCCCCTCTAGCGGGCGCGGGGCGAA
GCGGTGCGGCGCCGGCAGGAAGGAAATGGGCGGGGAGGGCCTTCGTGCGT
CGCCGCGCCGCCGTCCCCTTCTCCCTCTCCAGCCTCGGGGCTGTCCGCGG
GGGGACGGCTGCCTTCGGGGGGGACGGGGCAGGGCGGGGTTCGGCTTCTG
GCGTGTGACCGGCGGCTCTAGAGCCTCTGCTAACCATGTTCATGCCTTCT
TCTTTTTCCTACAGCTCCTGGGCAACGTGCTGGTTATTGTGCTGTCTCAT
CATTTTGGCAAAGAATTGCAAGTTTGTACAAAAAAGCAGGCTGCCACCAT
GTACAGGATGCAACTCCTGTCTTGCATTGCACTAAGTCTTGCACTTGTCA
CGAATTCGCAGGTGCAGCTGGTGCAGAGCGGCGTGGAAGTGAAAAAACCG
GGCGCGAGCGTGAAAGTGAGCTGCAAAGCGAGCGGCTATACCTTTACCAA
CTATTATATGTATTGGGTGCGCCAGGCGCCGGGCCAGGGCCTGGAATGGA
TGGGCGGCATTAACCCGAGCAACGGCGGCACCAACTTTAACGAAAAATTT
AAAAACCGCGTGACCCTGACCACCGATAGCAGCACCACCACCGCGTATAT
GGAACTGAAAAGCCTGCAGTTTGATGATACCGCGGTGTATTATTGCGCGC
GCCGCGATTATCGCTTTGATATGGGCTTTGATTATTGGGGCCAGGGCACC
ACCGTGACCGTGAGCAGCGCGAGCACCAAAGGCCCGAGCGTGTTTCCGCT
GGCGCCGTGCAGCCGCAGCACCAGCGAAAGCACCGCGGCGCTGGGCTGCC
TGGTGAAAGATTATTTTCCGGAACCGGTGACCGTGAGCTGGAACAGCGGC
GCGCTGACCAGCGGCGTGCATACCTTTCCGGCGGTGCTGCAGAGCAGCGG
CCTGTATAGCCTGAGCAGCGTGGTGACCGTGCCGAGCAGCAGCCTGGGCA
CCAAAACCTATACCTGCAACGTGGATCATAAACCGAGCAACACCAAAGTG
GATAAACGCGTGGAAAGCAAATATGGCCCGCCGTGCCCGCCGTGCCCGGC
GCCGGAATTTCTGGGCGGCCCGAGCGTGTTTCTGTTTCCGCCGAAACCGA
AAGATACCCTGATGATTAGCCGCACCCCGGAAGTGACCTGCGTGGTGGTG
GATGTGAGCCAGGAAGATCCGGAAGTGCAGTTTAACTGGTATGTGGATGG
CGTGGAAGTGCATAACGCGAAAACCAAACCGCGCGAAGAACAGTTTAACA
GCACCTATCGCGTGGTGAGCGTGCTGACCGTGCTGCATCAGGATTGGCTG
AACGGCAAAGAATATAAATGCAAAGTGAGCAACAAAGGCCTGCCGAGCAG
CATTGAAAAAACCATTAGCAAAGCGAAAGGCCAGCCGCGCGAACCGCAGG
TGTATACCCTGCCGCCGAGCCAGGAAGAAATGACCAAAAACCAGGTGAGC
CTGACCTGCCTGGTGAAAGGCTTTTATCCGAGCGATATTGCGGTGGAATG
GGAAAGCAACGGCCAGCCGGAAAACAACTATAAAACCACCCCGCCGGTGC
TGGATAGCGATGGCAGCTTTTTTCTGTATAGCCGCCTGACCGTGGATAAA
AGCCGCTGGCAGGAAGGCAACGTGTTTAGCTGCAGCGTGATGCATGAAGC
GCTGCATAACCATTATACCCAGAAAAGCCTGAGCCTGAGCCTGGGCAAAG
CTAGCACCAAGGGCCCATCGGTCTTCCCCCTGGCACCCTCCTCCAAGAGC
ACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGC
GTGGTGACCGTGCCCTCCAGCAGCTTGGGCACCCAGACCTACATCTGCAA
CGTGAATCACAAGCCCAGCAACACCAAGGTGGACAAGAAAGTTGAGCCCA
AATCTTGTGACAAAACTCACACATGCCCACCGTGCCCAGCACCTGAACTC
CTGGGGGGACCGTCAGTCTTCCTCTTCCCCCCAAAACCCAAGGACACCCT
CATGATCTCCCGGACCCCTGAGGTCACATGCGTGGTGGTGGACGTGAGCC
ACGAAGACCCTGAGGTCAAGTTCAACTGGTACGTGGACGGCGTGGAGGTG
CATAATGCCAAGACAAAGCCGCGGGAGGAGCAGTACAACAGCACGTACCG
TGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCT
GCCCCCATCCCGGGAGGAGATGACCAAGAACCAGGTCAGCCTGACCTGCC
TGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGC
AGCAGGGGAACGTCTTCTCATGCTCCGTGATGCATGAGGCTCTGCACAAC
CACTACACGCAGAAGAGCCTCTCCCTGTCTCCGGGTAAATAGACCCAGCT
TTCTTGTACAAAGTGGCTGTGCCTTCTAGTTGCCAGCCATCTGTTGTTTG
CCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCAT
TCTATTCTGGGGGGGGGGTGGGGCAGGACAGCAAGGGGGAGGATTGGGAA
GACAATAGCAGGCATGCTGGGGATGCGGTGGGCTCTATGGGGCTCCGGTG
CCCGTCAGTGGGCAGAGCGCACATCGCCCACAGTCCCCGAGAAGTTGGGG
GGAGGGGTCGGCAATTGAACCGGTGCCTAGAGAAGGTGGCGCGGGGTAAA
CTGGGAAAGTGATGTCGTGTACTGGCTCCGCCTTTTTCCCGAGGGTGGGG
GAGAACCGTATATAAGTGCAGTAGTCGCCGTGAACGTTCTTTTTCGCAAC
GGGTTTGCCGCCAGAACACAGGTAAGTGCCGTGTGTGGTTCCCGCGGGCC
TGGCCTCTTTACGGGTTATGGCCCTTGCGTGCCTTGAATTACTTCCACCT
GGCTGCAGTACGTGATTCTTGATCCCGAGCTTCGGGTTGGAAGTGGGTGG
GAGAGTTCGAGGCCTTGCGCTTAAGGAGCCCCTTCGCCTCGTGCTTGAGT
TGAGGCCTGGCCTGGGCGCTGGGGCCGCCGCGTGCGAATCTGGTGGCACC
TTCGCGCCTGTCTCGCTGCTTTCGATAAGTCTCTAGCCATTTAAAATTTT
TGATGACCTGCTGCGACGCTTTTTTTCTGGCAAGATAGTCTTGTAAATGC
GGGCCAAGATCTGCACACTGGTATTTCGGTTTTTGGGGCCGCGGGCGGCG
ACGGGGCCCGTGCGTCCCAGCGCACATGTTCGGCGAGGCGGGGCCTGCGA
GCGCGGCCACCGAGAATCGGACGGGGGTAGTCTCAAGCTGGCCGGCCTGC
TCTGGTGCCTGGTCTCGCGCCGCCGTGTATCGCCCCGCCCTGGGCGGCAA
GGCTGGCCCGGTCGGCACCAGTTGCGTGAGCGGAAAGATGGCCGCTTCCC
GGCCCTGCTGCAGGGAGCTCAAAATGGAGGACGCGGCGCTCGGGAGAGCG
GGCGGGTGAGTCACCCACACAAAGGAAAAGGGCCTTTCCGTCCTCAGCCG
TCGCTTCATGTGACTCCACGGAGTACCGGGCGCCGTCCAGGCACCTCGAT
TAGTTCTCGAGCTTTTGGAGTACGTCGTCTTTAGGTTGGGGGGAGGGGTT
TTATGCGATGGAGTTTCCCCACACTGAGTGGGTGGAGACTGAAGTTAGGC
CAGCTTGGCACTTGATGTAATTCTCCTTGGAATTTGCCCTTTTTGAGTTT
GGATCTTGGTTCATTCTCAAGCCTCAGACAGTGGTTCAAAGTTTTTTTCT
TCCATTTCAGGTGTCGTGAGCCACCATGTACAGGATGCAACTCCTGTCTT
GCATTGCACTAAGTCTTGCACTTGTCACGAATTCGGAAATTGTGCTGACC
CAGAGCCCGGCGACCCTGAGCCTGAGCCCGGGCGAACGCGCGACCCTGAG
CTGCCGCGCGAGCAAAGGCGTGAGCACCAGCGGCTATAGCTATCTGCATT
GGTATCAGCAGAAACCGGGCCAGGCGCCGCGCCTGCTGATTTATCTGGCG
AGCTATCTGGAAAGCGGCGTGCCGGCGCGCTTTAGCGGCAGCGGCAGCGG
CACCGATTTTACCCTGACCATTAGCAGCCTGGAACCGGAAGATTTTGCGG
TGTATTATTGCCAGCATAGCCGCGATCTGCCGCTGACCTTTGGCGGCGGC
ACCAAAGTGGAAATTAAACGCACCGTGGCGGCGCCGAGCGTGTTTATTTT
TCCGCCGAGCGATGAACAGCTGAAAAGCGGCACCGCGAGCGTGGTGTGCC
TGCTGAACAACTTTTATCCGCGCGAAGCGAAAGTGCAGTGGAAAGTGGAT
AACGCGCTGCAGAGCGGCAACAGCCAGGAAAGCGTGACCGAACAGGATAG
CAAAGATAGCACCTATAGCCTGAGCAGCACCCTGACCCTGAGCAAAGCGG
ATTATGAAAAACATAAAGTGTATGCGTGCGAAGTGACCCATCAGGGCCTG
AGCAGCCCGGTGACCAAAAGCTTTAACCGCGGCGAATGCACGGTGGCTGC
ACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAG
TGTCACAGAGCAGGACAGCAAGGACAGCACCTACAGCCTCAGCAGCACCC
TGACGCTGAGCAAAGCAGACTACGAGAAACACAAAGTCTACGCCTGCGAA
GTCACCCATCAGGGCCTGAGCTCGCCCGTCACAAAGAGCTTCAACAGGGG
AGAGTGTTAGCAACTTTATTATACATAGTTGATCCTCAGGTGCAGGCTGC
CTATCAGAAGGTGGTGGCTGGTGTGGCCAATGCCCTGGCTCACAAATACC
ACTGAGATCTTTTTCCCTCTGCCAAAAATTATGGGGACATCATGAAGCCC
CTTGAGCATCTGACTTCTGGCTAATAAAGGAAATTTATTTTCATTGCAAT
AGTGTGTTGGAATTTTTTGTGTCTCTCACTCGGAAGGACATATGGGAGGG
CAAATCATTTAAAACATCAGAATGAGTATTTGGTTTAGAGTTTGGCAACA
TATGCCCATATGCTGGCTGCCATGAACAAAGGTTGGCTATAAAGAGGTCA
TCAGTATATGAAACAGCCCCCTGCTGTCCATTCCTTATTCCATAGAAAAG
CCTTGACTTGAGGTTAGATTTTTTTTATATTTTGTTTTGTGTTATTTTTT
TCTTTAACATCCCTAAAATTTTCCTTACATGTTTTACTAGCCAGATTTTT
CCTCCTCTCCTGACTACTCCCAGTCATAGCTGTCCCTCTTCTCTTATGGA
GATCCCTCGACCTGCAGCCCAAGCTTCGCGTTGACATTGATTATTGACTA
GTTATTAATAGTAATCAATTACGGGGTCATTAGTTCATAGCCCATATATG
GAGTTCCGCGTTACATAACTTACGGTAAATGGCCCGCCTGGCTGACCGCC
CAACGACCCCCGCCCATTGACGTCAATAATGACGTATGTTCCCATAGTAA
CGCCAATAGGGACTTTCCATTGACGTCAATGGGTGGAGTATTTACGGTAA
ACTGCCCACTTGGCAGTACATCAAGTGTATCATATGCCAAGTACGCCCCC
TATTGACGTCAATGACGGTAAATGGCCCGCCTGGCATTATGCCCAGTACA
TGACCTTATGGGACTTTCCTACTTGGCAGTACATCTACGTATTAGTCATC
GCTATTACCATGGTGATGCGGTTTTGGCAGTACATCAATGGGCGTGGATA
GCGGTTTGACTCACGGGGATTTCCAAGTCTCCACCCCATTGACGTCAATG
GGAGTTTGTTTTGGCACCAAAATCAACGGGACTTTCCAAAATGTCGTAAC
AACTCCGCCCCATTGACGCAAATGGGCGGTAGGCGTGTACGGTGGGAGGT
CTATATAAGCAGAGCTCTCTGGCTAACTAGAGAACCCACTGCGCCACCAT
GACCGAGTACAAGCCCACGGTGCGCCTCGCCACCCGCGACGACGTCCCCA
GGGCCGTACGCACCCTCGCCGCCGCGTTCGCCGACTACCCCGCCACGCGC
CACACCGTCGATCCGGACCGCCACATCGAGCGGGTCACCGAGCTGCAAGA
ACTCTTCCTCACGCGCGTCGGGCTCGACATCGGCAAGGTGTGGGTCGCGG
ACGACGGCGCCGCGGTGGCGGTCTGGACCACGCCGGAGAGCGTCGAAGCG
GGGGCGGTGTTCGCCGAGATCGGCCCGCGCATGGCCGAGTTGAGCGGTTC
CCGGCTGGCCGCGCAGCAACAGATGGAAGGCCTCCTGGCGCCGCACCGGC
CCAAGGAGCCCGCGTGGTTCCTGGCCACCGTCGGCGTCTCGCCCGACCAC
CAGGGCAAGGGTCTGGGCAGCGCCGTCGTGCTCCCCGGAGTGGAGGCGGC
CGAGCGCGCCGGGGTGCCCGCCTTCCTGGAGACCTCCGCGCCCCGCAACC
TCCCCTTCTACGAGCGGCTCGGCTTCACCGTCACCGCCGACGTCGAGGTG
CCCGAAGGACCGCGCACCTGGTGCATGACCCGCAAGCCCGGTGCCTGACT
CGAGTCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGAT
GCGGTGGGCTCTATGGCTCGAGTTAATTAACGAGAGCATAATATTGATAT
GTGCCAAAGTTGTTTCTGACTGACTAATAAGTATAATTTGTTTCTATTAT
GTATAGGTTAAGCTAATTACTTATTTTATAATACAACATGACTGTTTTTA
AAGTACAAAATAAGTTTATTTTTGTAAAAGAGAGAATGTTTAAAAGTTTT
GTTACTTTATAGAAGAAATTTTGAGTTTTTGTTTTTTTTTAATAAATAAA
TAAACATAAATAAATTGTTTGTTGAATTTATTATTAGTATGTAAGTGTAA
ATATAATAAAACTTAATATCTATTCAAATTAATAAATAAACCTCGATATA
CAGACCGATAAAACACATGCGTCAATTTTACGCATGATTATCTTTAACGT
ACGTCACAATATGATTATCTTTCTAGGGTTAAATAATAGTTTCTAATTTT
TTTATTATTCAGCCTGCTGTCGTGAATACCGAGCTCCAATTCGCCCTATA
GTGAGTCGTATTACAATTCACTGGCCGTCGTTTTACAACGTCGTGACTGG
GAAAACCCTGGCGTTACCCAACTTAATCGCCTTGCAGCACATCCCCCTTT
CGCCAGCTGGCGTAATAGCGAAGAGGCCCGCACCGATCGCCCTTCCCAAC
AGTTGCGCAGCCTGAATGGCGAATGGGACGCGCCCTGTAGCGGCGCATTA
AGCGCGGCGGGTGTGGTGGTTACGCGCAGCGTGACCGCTACACTTGCCAG
CGCCCTAGCGCCCGCTCCTTTCGCTTTCTTCCCTTCCTTTCTCGCCACGT
TCGCCGGCTTTCCCCGTCAAGCTCTAAATCGGGGGCTCCCTTTAGGGTTC
CGATTTAGTGCTTTACGGCACCTCGACCCCAAAAAACTTGATTAGGGTGA
TGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACA
ACACTCAACCCTATCTCGGTCTATTCTTTTGATTTATAAGGGATTTTGCC
GATTTCGGCCTATTGGTTAAAAAATGAGCTGATTTAACAAAAATTTAACG
CGAATTTTAACAAAATATTAACGCTTACAATTTAGGTGGCACTTTTCGGG
GAAATGTGCGCGGAACCCCTATTTGTTTATTTTTCTAAATACATTCAAAT
ATGTATCCGCTCATGAGACAATAACCCTGATAAATGCTTCAATAATATTG
AAAAAGGAAGAGTATGAGTATTCAACATTTCCGTGTCGCCCTTATTCCCT
TTTTTGCGGCATTTTGCCTTCCTGTTTTTGCTCACCCAGAAACGCTGGTG
AAAGTAAAAGATGCTGAAGATCAGTTGGGTGCACGAGTGGGTTACATCGA
ACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTA
TCCCGTATTGACGCCGGGCAAGAGCAACTCGGTCGCCGCATACACTATTC
TCAGAATGACTTGGTTGAGTACTCACCAGTCACAGAAAAGCATCTTACGG
ATGGCATGACAGTAAGAGAATTATGCAGTGCTGCCATAACCATGAGTGAT
AACACTGCGGCCAACTTACTTCTGACAACGATCGGAGGACCGAAGGAGCT
AACCGCTTTTTTGCACAACATGGGGGATCATGTAACTCGCCTTGATCGTT
GGGAACCGGAGCTGAATGAAGCCATACCAAACGACGAGCGTGACACCACG
ATGCCTGTAGCAATGGCAACAACGTTGCGCAAACTATTAACTGGCGAACT
ACTTACTCTAGCTTCCCGGCAACAATTAATAGACTGGATGGAGGCGGATA
AAGTTGCAGGACCACTTCTGCGCTCGGCCCTTCCGGCTGGCTGGTTTATT
GCTGATAAATCTGGAGCCGGTGAGCGTGGGTCTCGCGGTATCATTGCAGC
ACTGGGGCCAGATGGTAAGCCCTCCCGTATCGTAGTTATCTACACGACGG
GGAGTCAGGCAACTATGGATGAACGAAATAGACAGATCGCTGAGATAGGT
GCCTCACTGATTAAGCATTGGTAACTGTCAGACCAAGTTTACTCATATAT
ACTTTAGATTGATTTAAAACTTCATTTTTAATTTAAAAGGATCTAGGTGA
AGATCCTTTTTGATAATCTCATGACCAAAATCCCTTAACGTGAGTTTTCG
TTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCG
AAGGTAACTGGCTTCAGCAGAGCGCAGATACCAAATACTGTTCTTCTAGT
GTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAA
CGACCTACACCGAACTGAGATACCTACAGCGTGAGCTATGAGAAAGCGCC
ACGCTTCCCGAAGGGAGAAAGGCGGACAGGTATCCGGTAAGCGGCAGGGT
CGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTG
TGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTC
CTGCGTTATCCCCTGATTCTGTGGATAACCGTATTACCGCCTTTGAGTGA
GCTGATACCGCTCGCCGCAGCCGAACGACCGAGCGCAGCGAGTCAGTGAG
CGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCG
GGCAGTGAGCGCAACGCAATTAATGTGAGTTAGCTCACTCATTAGGCACC
CCAGGCTTTACACTTTATGCTTCCGGCTCGTATGTTGTGTGGAATTGTGA
GCGGATAACAATTTCACACAGGAAACAGCTATGACCATGATTACGCCAAG
CTCGAAATTAACCCTCACTAAAGGGAACAAAAGCTGGTACCTCGCGCGAC
TTGGTTTGCCATTCTTTAGCGCGCGTCGCGTCACACAGCTTGGCCACAAT
GTGGTTTTTGTCAAACGAAGATTCTATGACGTGTTTAAAGTTTAGGTCGA
GTAAAGCGCAAATCTTTT

In some embodiments, the anti-PD-1 antibody is encoded by the aPD1 CAG_EF1a_CMV_hlgy2-CL (human) plasmid comprising the nucleic acid sequence of:

(SEQ ID NO: 21)
TTAACCCTAGAAAGATAGTCTGCGTAAAATTGACGCATGCATTCTTGAAA
TATTGCTCTCTCTTTCTAAATAGCGCGAATCCGTCGCTGTGCATTTAGGA
CATCTCAGTCGCCGCTTGGAGCTCCCGTGAGGCGTGCTTGTCAATGCGGT
AAGTGTCACTGATTTTGAACTATAACGACCGCGTGAGTCAAAATGACGCA
TGATTATCTTTTACGTGACTTTTAAGATTTAACTCATACGATAATTATAT
TGTTATTTCATGTTCTACTTACGTGATAACTTATTATATATATATTTTCT
TGTTATAGATATCATCAACTTTGTATAGAAAAGTTGCTCGACATTGATTA
TTGACTAGTTATTAATAGTAATCAATTACGGGGTCATTAGTTCATAGCCC
ATATATGGAGTTCCGCGTTACATAACTTACGGTAAATGGCCCGCCTGGCT
GACCGCCCAACGACCCCCGCCCATTGACGTCAATAATGACGTATGTTCCC
ATAGTAACGCCAATAGGGACTTTCCATTGACGTCAATGGGTGGAGTATTT
ACGGTAAACTGCCCACTTGGCAGTACATCAAGTGTATCATATGCCAAGTA
CGCCCCCTATTGACGTCAATGACGGTAAATGGCCCGCCTGGCATTATGCC
CAGTACATGACCTTATGGGACTTTCCTACTTGGCAGTACATCTACGTATT
AGTCATCGCTATTACCATGGTCGAGGTGAGCCCCACGTTCTGCTTCACTC
TCCCCATCTCCCCCCCCTCCCCACCCCCAATTTTGTATTTATTTATTTTT
TAATTATTTTGTGCAGCGATGGGGGGGGGGGGGGGGGGGGGGGGCGCGCC
AGGCGGGGCGGGGCGGGGCGAGGGGCGGGGCGGGGCGAGGCGGAGAGGTG
CGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGG
AGTCGCTGCGCGCTGCCTTCGCCCCGTGCCCCGCTCCGCCGCCGCCTCGC
GCCGCCCGCCCCGGCTCTGACTGACCGCGTTACTCCCACAGGTGAGCGGG
CGGGACGGCCCTTCTCCTCCGGGCTGTAATTAGCGCTTGGTTTAATGACG
GCTTGTTTCTTTTCTGTGGCTGCGTGAAAGCCTTGAGGGGCTCCGGGAGG
GCCCTTTGTGCGGGGGGAGCGGCTCGGGGGGTGCGTGCGTGTGTGTGTGC
GTGGGGAGCGCCGCGTGCGGCTCCGCGCTGCCCGGCGGCTGTGAGCGCTG
CGGGCGCGGCGCGGGGCTTTGTGCGCTCCGCAGTGTGCGCGAGGGGAGCG
CGGCCGGGGGCGGTGCCCCGCGGTGCGGGGGGGGCTGCGAGGGGAACAAA
GGCTGCGTGCGGGGTGTGTGCGTGGGGGGGTGAGCAGGGGGTGTGGGCGC
GTCGGTCGGGCTGCAACCCCCCCTGCACCCCCCTCCCCGAGTTGCTGAGC
ACGGCCCGGCTTCGGGTGCGGGGCTCCGTACGGGGCGTGGCGCGGGGCTC
GCCGTGCCGGGCGGGGGGTGGCGGCAGGTGGGGGTGCCGGGCGGGGCGGG
GCCGCCTCGGGCCGGGGAGGGCTCGGGGGAGGGGCGCGGCGGCCCCCGGA
GCGCCGGCGGCTGTCGAGGCGCGGCGAGCCGCAGCCATTGCCTTTTATGG
TAATCGTGCGAGAGGGCGCAGGGACTTCCTTTGTCCCAAATCTGTGCGGA
GCCGAAATCTGGGAGGCGCCGCCGCACCCCCTCTAGCGGGCGCGGGGCGA
AGCGGTGCGGCGCCGGCAGGAAGGAAATGGGCGGGGAGGGCCTTCGTGCG
TCGCCGCGCCGCCGTCCCCTTCTCCCTCTCCAGCCTCGGGGCTGTCCGCG
GGGGGACGGCTGCCTTCGGGGGGGACGGGGCAGGGCGGGGTTCGGCTTCT
GGCGTGTGACCGGCGGCTCTAGAGCCTCTGCTAACCATGTTCATGCCTTC
TTCTTTTTCCTACAGCTCCTGGGCAACGTGCTGGTTATTGTGCTGTCTCA
TCATTTTGGCAAAGAATTGCAAGTTTGTACAAAAAAGCAGGCTGCCACCA
TGTACAGGATGCAACTCCTGTCTTGCATTGCACTAAGTCTTGCACTTGTC
ACGAATTCGCAGGTGCAGCTGGTGCAGAGCGGCGTGGAAGTGAAAAAACC
GGGCGCGAGCGTGAAAGTGAGCTGCAAAGCGAGCGGCTATACCTTTACCA
ACTATTATATGTATTGGGTGCGCCAGGCGCCGGGCCAGGGCCTGGAATGG
ATGGGCGGCATTAACCCGAGCAACGGCGGCACCAACTTTAACGAAAAATT
TAAAAACCGCGTGACCCTGACCACCGATAGCAGCACCACCACCGCGTATA
TGGAACTGAAAAGCCTGCAGTTTGATGATACCGCGGTGTATTATTGCGCG
CGCCGCGATTATCGCTTTGATATGGGCTTTGATTATTGGGGCCAGGGCAC
CACCGTGACCGTGAGCAGCGCGAGCACCAAAGGCCCGAGCGTGTTTCCGC
TGGCGCCGTGCAGCCGCAGCACCAGCGAAAGCACCGCGGCGCTGGGCTGC
CTGGTGAAAGATTATTTTCCGGAACCGGTGACCGTGAGCTGGAACAGCGG
CGCGCTGACCAGCGGCGTGCATACCTTTCCGGCGGTGCTGCAGAGCAGCG
GCCTGTATAGCCTGAGCAGCGTGGTGACCGTGCCGAGCAGCAGCCTGGGC
ACCAAAACCTATACCTGCAACGTGGATCATAAACCGAGCAACACCAAAGT
GGATAAACGCGTGGAAAGCAAATATGGCCCGCCGTGCCCGCCGTGCCCGG
CGCCGGAATTTCTGGGCGGCCCGAGCGTGTTTCTGTTTCCGCCGAAACCG
AAAGATACCCTGATGATTAGCCGCACCCCGGAAGTGACCTGCGTGGTGGT
GGATGTGAGCCAGGAAGATCCGGAAGTGCAGTTTAACTGGTATGTGGATG
GCGTGGAAGTGCATAACGCGAAAACCAAACCGCGCGAAGAACAGTTTAAC
AGCACCTATCGCGTGGTGAGCGTGCTGACCGTGCTGCATCAGGATTGGCT
GAACGGCAAAGAATATAAATGCAAAGTGAGCAACAAAGGCCTGCCGAGCA
GCATTGAAAAAACCATTAGCAAAGCGAAAGGCCAGCCGCGCGAACCGCAG
GTGTATACCCTGCCGCCGAGCCAGGAAGAAATGACCAAAAACCAGGTGAG
CCTGACCTGCCTGGTGAAAGGCTTTTATCCGAGCGATATTGCGGTGGAAT
GGGAAAGCAACGGCCAGCCGGAAAACAACTATAAAACCACCCCGCCGGTG
CTGGATAGCGATGGCAGCTTTTTTCTGTATAGCCGCCTGACCGTGGATAA
AAGCCGCTGGCAGGAAGGCAACGTGTTTAGCTGCAGCGTGATGCATGAAG
CGCTGCATAACCATTATACCCAGAAAAGCCTGAGCCTGAGCCTGGGCAAA
GCTAGCACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAAAGTTGAGCCC
AAATCTTGTGACAAAACTCACACATGCCCACCGTGCCCAGCACCTGAACT
CCTGGGGGGACCGTCAGTCTTCCTCTTCCCCCCAAAACCCAAGGACACCC
TCATGATCTCCCGGACCCCTGAGGTCACATGCGTGGTGGTGGACGTGAGC
CACGAAGACCCTGAGGTCAAGTTCAACTGGTACGTGGACGGCGTGGAGGT
GCATAATGCCAAGACAAAGCCGCGGGAGGAGCAGTACAACAGCACGTACC
GTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCC
TGCCCCCATCCCGGGAGGAGATGACCAAGAACCAGGTCAGCCTGACCTGC
CTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCG
ACGGCTCCTTCTTCCTCTACAGCAAGCTCACCGTGGACAAGAGCAGGTGG
CAGCAGGGGAACGTCTTCTCATGCTCCGTGATGCATGAGGCTCTGCACAA
CCACTACACGCAGAAGAGCCTCTCCCTGTCTCCGGGTAAATAGACCCAGC
TTTCTTGTACAAAGTGGCTGTGCCTTCTAGTTGCCAGCCATCTGTTGTTT
GCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGG
AAGACAATAGCAGGCATGCTGGGGATGCGGTGGGCTCTATGGGGCTCCGG
TGCCCGTCAGTGGGCAGAGCGCACATCGCCCACAGTCCCCGAGAAGTTGG
GGGGAGGGGTCGGCAATTGAACCGGTGCCTAGAGAAGGTGGCGCGGGGTA
AACTGGGAAAGTGATGTCGTGTACTGGCTCCGCCTTTTTCCCGAGGGTGG
GGGAGAACCGTATATAAGTGCAGTAGTCGCCGTGAACGTTCTTTTTCGCA
ACGGGTTTGCCGCCAGAACACAGGTAAGTGCCGTGTGTGGTTCCCGCGGG
CCTGGCCTCTTTACGGGTTATGGCCCTTGCGTGCCTTGAATTACTTCCAC
CTGGCTGCAGTACGTGATTCTTGATCCCGAGCTTCGGGTTGGAAGTGGGT
GGGAGAGTTCGAGGCCTTGCGCTTAAGGAGCCCCTTCGCCTCGTGCTTGA
GTTGAGGCCTGGCCTGGGCGCTGGGGCCGCCGCGTGCGAATCTGGTGGCA
CCTTCGCGCCTGTCTCGCTGCTTTCGATAAGTCTCTAGCCATTTAAAATT
TTTGATGACCTGCTGCGACGCTTTTTTTCTGGCAAGATAGTCTTGTAAAT
GCGGGCCAAGATCTGCACACTGGTATTTCGGTTTTTGGGGCCGCGGGCGG
CGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCT
GCTCTGGTGCCTGGTCTCGCGCCGCCGTGTATCGCCCCGCCCTGGGCGGC
AAGGCTGGCCCGGTCGGCACCAGTTGCGTGAGCGGAAAGATGGCCGCTTC
CCGGCCCTGCTGCAGGGAGCTCAAAATGGAGGACGCGGCGCTCGGGAGAG
CGGGCGGGTGAGTCACCCACACAAAGGAAAAGGGCCTTTCCGTCCTCAGC
CGTCGCTTCATGTGACTCCACGGAGTACCGGGCGCCGTCCAGGCACCTCG
ATTAGTTCTCGAGCTTTTGGAGTACGTCGTCTTTAGGTTGGGGGGAGGGG
TTTTATGCGATGGAGTTTCCCCACACTGAGTGGGTGGAGACTGAAGTTAG
GCCAGCTTGGCACTTGATGTAATTCTCCTTGGAATTTGCCCTTTTTGAGT
TTGGATCTTGGTTCATTCTCAAGCCTCAGACAGTGGTTCAAAGTTTTTTT
CTTCCATTTCAGGTGTCGTGAGCCACCATGTACAGGATGCAACTCCTGTC
TTGCATTGCACTAAGTCTTGCACTTGTCACGAATTCGGAAATTGTGCTGA
CCCAGAGCCCGGCGACCCTGAGCCTGAGCCCGGGCGAACGCGCGACCCTG
AGCTGCCGCGCGAGCAAAGGCGTGAGCACCAGCGGCTATAGCTATCTGCA
TTGGTATCAGCAGAAACCGGGCCAGGCGCCGCGCCTGCTGATTTATCTGG
CGAGCTATCTGGAAAGCGGCGTGCCGGCGCGCTTTAGCGGCAGCGGCAGC
GGCACCGATTTTACCCTGACCATTAGCAGCCTGGAACCGGAAGATTTTGC
GGTGTATTATTGCCAGCATAGCCGCGATCTGCCGCTGACCTTTGGCGGCG
GCACCAAAGTGGAAATTAAACGCACCGTGGCGGCGCCGAGCGTGTTTATT
TTTCCGCCGAGCGATGAACAGCTGAAAAGCGGCACCGCGAGCGTGGTGTG
CCTGCTGAACAACTTTTATCCGCGCGAAGCGAAAGTGCAGTGGAAAGTGG
ATAACGCGCTGCAGAGCGGCAACAGCCAGGAAAGCGTGACCGAACAGGAT
AGCAAAGATAGCACCTATAGCCTGAGCAGCACCCTGACCCTGAGCAAAGC
GGATTATGAAAAACATAAAGTGTATGCGTGCGAAGTGACCCATCAGGGCC
TGAGCAGCCCGGTGACCAAAAGCTTTAACCGCGGCGAATGCGGTCAGCCC
AAGGCTGCCCCCTCGGTCACTCTGTTCCCGCCCTCCTCTGAGGAGCTTCA
AGCCAACAAGGCCACACTGGTGTGTCTCATAAGTGACTTCTACCCGGGAG
CCGTGACAGTGGCCTGGAAGGCAGATAGCAGCCCCGTCAAGGCGGGAGTG
GAGACCACCACACCCTCCAAACAAAGCAACAACAAGTACGCGGCCAGCAG
CTATCTGAGCCTGACGCCTGAGCAGTGGAAGTCCCACAGAAGCTACAGCT
GCCAGGTCACGCATGAAGGGAGCACCGTGGAGAAGACAGTGGCCCCTACA
GAATGTTCATAGCAACTTTATTATACATAGTTGATCCTCAGGTGCAGGCT
GCCTATCAGAAGGTGGTGGCTGGTGTGGCCAATGCCCTGGCTCACAAATA
CCACTGAGATCTTTTTCCCTCTGCCAAAAATTATGGGGACATCATGAAGC
CCCTTGAGCATCTGACTTCTGGCTAATAAAGGAAATTTATTTTCATTGCA
ATAGTGTGTTGGAATTTTTTGTGTCTCTCACTCGGAAGGACATATGGGAG
GGCAAATCATTTAAAACATCAGAATGAGTATTTGGTTTAGAGTTTGGCAA
CATATGCCCATATGCTGGCTGCCATGAACAAAGGTTGGCTATAAAGAGGT
CATCAGTATATGAAACAGCCCCCTGCTGTCCATTCCTTATTCCATAGAAA
AGCCTTGACTTGAGGTTAGATTTTTTTTATATTTTGTTTTGTGTTATTTT
TTTCTTTAACATCCCTAAAATTTTCCTTACATGTTTTACTAGCCAGATTT
TTCCTCCTCTCCTGACTACTCCCAGTCATAGCTGTCCCTCTTCTCTTATG
GAGATCCCTCGACCTGCAGCCCAAGCTTCGCGTTGACATTGATTATTGAC
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTCTCTGGCTAACTAGAGAACCCACTGCGCCACC
ATGACCGAGTACAAGCCCACGGTGCGCCTCGCCACCCGCGACGACGTCCC
CAGGGCCGTACGCACCCTCGCCGCCGCGTTCGCCGACTACCCCGCCACGC
GCCACACCGTCGATCCGGACCGCCACATCGAGCGGGTCACCGAGCTGCAA
GAACTCTTCCTCACGCGCGTCGGGCTCGACATCGGCAAGGTGTGGGTCGC
GGACGACGGCGCCGCGGTGGCGGTCTGGACCACGCCGGAGAGCGTCGAAG
CGGGGGCGGTGTTCGCCGAGATCGGCCCGCGCATGGCCGAGTTGAGCGGT
TCCCGGCTGGCCGCGCAGCAACAGATGGAAGGCCTCCTGGCGCCGCACCG
GCCCAAGGAGCCCGCGTGGTTCCTGGCCACCGTCGGCGTCTCGCCCGACC
ACCAGGGCAAGGGTCTGGGCAGCGCCGTCGTGCTCCCCGGAGTGGAGGCG
GCCGAGCGCGCCGGGGTGCCCGCCTTCCTGGAGACCTCCGCGCCCCGCAA
CCTCCCCTTCTACGAGCGGCTCGGCTTCACCGTCACCGCCGACGTCGAGG
TGCCCGAAGGACCGCGCACCTGGTGCATGACCCGCAAGCCCGGTGCCTGA
CTCGAGTCTAGAGGGCCCGTTTAAACCCGCTGATCAGCCTCGACTGTGCC
TTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGG
GCAGGACAGCAAGGGGGAGGATTGGGAAGACAATAGCAGGCATGCTGGGG
ATGCGGTGGGCTCTATGGCTCGAGTTAATTAACGAGAGCATAATATTGAT
ATGTGCCAAAGTTGTTTCTGACTGACTAATAAGTATAATTTGTTTCTATT
ATGTATAGGTTAAGCTAATTACTTATTTTATAATACAACATGACTGTTTT
TAAAGTACAAAATAAGTTTATTTTTGTAAAAGAGAGAATGTTTAAAAGTT
TTGTTACTTTATAGAAGAAATTTTGAGTTTTTGTTTTTTTTTAATAAATA
AATAAACATAAATAAATTGTTTGTTGAATTTATTATTAGTATGTAAGTGT
AAATATAATAAAACTTAATATCTATTCAAATTAATAAATAAACCTCGATA
TACAGACCGATAAAACACATGCGTCAATTTTACGCATGATTATCTTTAAC
GTACGTCACAATATGATTATCTTTCTAGGGTTAAATAATAGTTTCTAATT
TTTTTATTATTCAGCCTGCTGTCGTGAATACCGAGCTCCAATTCGCCCTA
TAGTGAGTCGTATTACAATTCACTGGCCGTCGTTTTACAACGTCGTGACT
GGGAAAACCCTGGCGTTACCCAACTTAATCGCCTTGCAGCACATCCCCCT
TTCGCCAGCTGGCGTAATAGCGAAGAGGCCCGCACCGATCGCCCTTCCCA
ACAGTTGCGCAGCCTGAATGGCGAATGGGACGCGCCCTGTAGCGGCGCAT
TAAGCGCGGCGGGTGTGGTGGTTACGCGCAGCGTGACCGCTACACTTGCC
AGCGCCCTAGCGCCCGCTCCTTTCGCTTTCTTCCCTTCCTTTCTCGCCAC
GTTCGCCGGCTTTCCCCGTCAAGCTCTAAATCGGGGGCTCCCTTTAGGGT
TCCGATTTAGTGCTTTACGGCACCTCGACCCCAAAAAACTTGATTAGGGT
GATGGTTCACGTAGTGGGCCATCGCCCTGATAGACGGTTTTTCGCCCTTT
GACGTTGGAGTCCACGTTCTTTAATAGTGGACTCTTGTTCCAAACTGGAA
CAACACTCAACCCTATCTCGGTCTATTCTTTTGATTTATAAGGGATTTTG
CCGATTTCGGCCTATTGGTTAAAAAATGAGCTGATTTAACAAAAATTTAA
CGCGAATTTTAACAAAATATTAACGCTTACAATTTAGGTGGCACTTTTCG
GGGAAATGTGCGCGGAACCCCTATTTGTTTATTTTTCTAAATACATTCAA
ATATGTATCCGCTCATGAGACAATAACCCTGATAAATGCTTCAATAATAT
TGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGA
ACGTTTTCCAATGATGAGCACTTTTAAAGTTCTGCTATGTGGCGCGGTAT
TATCCCGTATTGACGCCGGGCAAGAGCAACTCGGTCGCCGCATACACTAT
TCTCAGAATGACTTGGTTGAGTACTCACCAGTCACAGAAAAGCATCTTAC
GGATGGCATGACAGTAAGAGAATTATGCAGTGCTGCCATAACCATGAGTG
ATAACACTGCGGCCAACTTACTTCTGACAACGATCGGAGGACCGAAGGAG
CTAACCGCTTTTTTGCACAACATGGGGGATCATGTAACTCGCCTTGATCG
TTGGGAACCGGAGCTGAATGAAGCCATACCAAACGACGAGCGTGACACCA
CGATGCCTGTAGCAATGGCAACAACGTTGCGCAAACTATTAACTGGCGAA
CTACTTACTCTAGCTTCCCGGCAACAATTAATAGACTGGATGGAGGCGGA
TAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCA
GCACTGGGGCCAGATGGTAAGCCCTCCCGTATCGTAGTTATCTACACGAC
GGGGAGTCAGGCAACTATGGATGAACGAAATAGACAGATCGCTGAGATAG
GTGCCTCACTGATTAAGCATTGGTAACTGTCAGACCAAGTTTACTCATAT
ATACTTTAGATTGATTTAAAACTTCATTTTTAATTTAAAAGGATCTAGGT
GAAGATCCTTTTTGATAATCTCATGACCAAAATCCCTTAACGTGAGTTTT
CGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTC
CGAAGGTAACTGGCTTCAGCAGAGCGCAGATACCAAATACTGTTCTTCTA
GTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATA
AGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCG
AACGACCTACACCGAACTGAGATACCTACAGCGTGAGCTATGAGAAAGCG
CCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTT
TGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTT
TCCTGCGTTATCCCCTGATTCTGTGGATAACCGTATTACCGCCTTTGAGT
GAGCTGATACCGCTCGCCGCAGCCGAACGACCGAGCGCAGCGAGTCAGTG
AGCGAGGAAGCGGAAGAGCGCCCAATACGCAAACCGCCTCTCCCCGCGCG
TTGGCCGATTCATTAATGCAGCTGGCACGACAGGTTTCCCGACTGGAAAG
CGGGCAGTGAGCGCAACGCAATTAATGTGAGTTAGCTCACTCATTAGGCA
CCCCAGGCTTTACACTTTATGCTTCCGGCTCGTATGTTGTGTGGAATTGT
GAGCGGATAACAATTTCACACAGGAAACAGCTATGACCATGATTACGCCA
AGCTCGAAATTAACCCTCACTAAAGGGAACAAAAGCTGGTACCTCGCGCG
ACTTGGTTTGCCATTCTTTAGCGCGCGTCGCGTCACACAGCTTGGCCACA
ATGTGGTTTTTGTCAAACGAAGATTCTATGACGTGTTTAAAGTTTAGGTC
GAGTAAAGCGCAAATCTTTT

In some embodiments, the immune checkpoint inhibitor may be an anti-LAG-3 antibody. LAG-3 (see, e.g., GenBank Accession No. NP-002277) is a protein on the surface of activated T-cells, natural killer cells, B-cells, and plasmacytoid dendritic cells. LAG-3 binds MHC class II molecules on the surface of antigen-presenting cells. Upon binding, LAG-3 transmits an inhibitory signal that deactivates effector T cells and inhibitor regulatory T cells. In some embodiments, the anti-LAG-3 antibody is relatlimab (see, e.g., International Patent Application Pub. No. WO 2015/116539), TSR-033 (see, e.g., International Patent Application Pub. No. WO 2018/201096), MK-4280, or REGN3767.

As provided for herein, the compositions may comprise a cell expressing a single type of protein of interest, e.g., a single protein or nucleic acid, or may express more than one type of protein of interest, e.g., a plurality of proteins or nucleic acids. In some embodiments, a composition comprises a cell expressing two types of proteins of interest (e.g., two types of proteins or nucleic acids). In some embodiments, a composition comprises a cell expressing three types of proteins of interest (e.g., three types of proteins or nucleic acids). In some embodiments, a composition comprises a cell expressing four types of proteins of interest (e.g., four types of proteins or nucleic acids).

In some embodiments, the composition comprises a cell expressing a single type of nucleic acid (e.g., DNA or RNA) or more than one type of nucleic acid, e.g., a plurality of nucleic acid (e.g., DNA or RNA). In some embodiments, the composition comprises a cell expressing two types of nucleic acids (e.g., DNA or RNA). In some embodiments, the composition comprises a cell expressing three types of nucleic acids (e.g., DNA or RNA). In some embodiments, the composition comprises a cell expressing four types of nucleic acids (e.g., DNA or RNA).

In some embodiments, the composition comprises a cell expressing a single type of protein, or more than one type of protein, e.g., a plurality of proteins. In some embodiments, the composition comprises a cell expressing two types of proteins. In some embodiments, the composition comprises a cell expressing three types of proteins. In some embodiments, the composition comprises a cell expressing four types of proteins.

In some embodiments, the composition comprises a cell expressing a single type of enzyme, or may express more than one type of enzyme, e.g., a plurality of enzymes. In some embodiments, a composition comprises a cell expressing two types of enzymes. In some embodiments, a composition comprises a cell expressing three types of enzymes. In some embodiments, a composition comprises a cell expressing four types of enzymes.

In some embodiments, the composition comprises a cell expressing a single type of cytokine or more than one type of cytokine, e.g., a plurality of cytokines. In some embodiments, the composition comprises a cell expressing two types of cytokines. In some embodiments, the composition comprises a cell expressing three types of cytokines. In some embodiments, the composition comprises a cell expressing four types of cytokines.

In some embodiments, the composition comprises a cell expressing a single type of antibody or antibody fragment or more than one type of antibody or antibody fragment, e.g., a plurality of antibodies or antibody fragments. In some embodiments, the composition comprises a cell expressing two types of antibodies or antibody fragments. In some embodiments, the composition comprises a cell expressing three types of antibodies or antibody fragments. In some embodiments, the composition comprises a cell expressing four types of antibodies or antibody fragments.

In some embodiments, the composition comprises a plurality of cells expressing a single type of cytokine or more than one type of cytokine, e.g., a plurality of cytokines; and a single type of antibody, or antibody fragment, or more than one type of antibody or antibody fragment, e.g., a plurality of antibodies or antibody fragments. In some embodiments, the composition comprises a plurality of cells expressing two types of cytokines, and two types of antibodies or antibody fragments. In some embodiments, the composition comprises a plurality of cells expressing three types of cytokines, and three types of antibodies or antibody fragments. In some embodiments, the composition comprises a plurality of cells expressing four types of cytokines, and four types of antibodies, or antibody fragments.

In some embodiments, the composition comprises a plurality of cells expressing IL-2, IL-12, IFN-α, IFN-γ, or IL-15, and an anti-PD-1 antibody, anti-PD-L1 antibody, anti-CTLA4 antibody, or anti-LAG-3 antibody, or antibody fragment thereof. In some embodiments, the composition comprises a plurality of cells expressing IL-2, and an anti-PD-L1 antibody, or antibody fragment thereof. In some embodiments, the composition comprises a plurality of cells expressing IL-2, and an anti-CTLA4 antibody, or antibody fragment thereof. In some embodiments, the composition comprises a plurality of cells expressing IL-2, and an anti-LAG-3 antibody, or antibody fragment thereof. In some embodiments, the composition comprises a plurality of cells expressing IL-12, and an anti-PD-L1 antibody, or antibody fragment thereof. In some embodiments, the composition comprises a plurality of cells expressing IL-12, and an anti-CTLA4 antibody, or antibody fragment thereof. In some embodiments, the composition comprises a plurality of cells expressing IL-12, and an anti-LAG-3 antibody, or antibody fragment thereof. In some embodiments, the composition comprises a plurality of cells expressing IFN-α, and an anti-PD-L1 antibody, or antibody fragment thereof. In some embodiments, the composition comprises a plurality of cells expressing IFN-α, and an anti-CTLA4 antibody, or antibody fragment thereof. In some embodiments, the composition comprises a plurality of cells expressing IFN-α, and an anti-LAG-3 antibody, or antibody fragment thereof. In some embodiments, the composition comprises a plurality of cells expressing IFN-γ, and an anti-PD-L1 antibody, or antibody fragment thereof. In some embodiments, the composition comprises a plurality of cells expressing IFN-γ, and an anti-CTLA4 antibody, or antibody fragment thereof. In some embodiments, the composition comprises a plurality of cells expressing IFN-γ, and an anti-LAG-3 antibody, or antibody fragment thereof. In some embodiments, the composition comprises a plurality of cells expressing IL-15, and an anti-PD-L1 antibody, or antibody fragment thereof. In some embodiments, the composition comprises a plurality of cells expressing IL-15, and an anti-CTLA4 antibody, or antibody fragment thereof. In some embodiments, the composition comprises a plurality of cells expressing IL-15, and an anti-LAG-3 antibody, or antibody fragment thereof.

### D. Inducible Kill Switch

A cell may be engineered to comprise a small molecule inducible kill switch, which may provide precise coordination of an immune response. A kill switch may be added to enhance the safety of cells engineered to produce cytokines. In some embodiments, the kill switch may comprise two engineered proteins may be located inside the cell, and dimerize upon exposure to a small molecule drug called rimiducid through a process referred to as chemical induction of dimerization (CID). CID with small molecules is an effective technology used to generate switches of protein function to alter cell physiology. AP1903 (rimiducid) is a high-specificity, efficient dimerizer which has two identical, protein-binding surfaces arranged tail-to-tail, each with high affinity and specificity for a mutant or variant of FKBP12: FKBP12 (F36V) (FKBP12v36, FV36 or FV). Attachment of one or more FV domains onto one or more cell-signaling molecules that normally rely on homodimerization can place that protein under rimiducid control. A molecular switch like rimiducid activates pro-apoptotic polypeptide Caspase 9, inducing cell death. A “kill switch” can be any modality that can be activated upon the addition of an exogenous factor to either turn off the expression of a protein of interest or to kill the cell, either through apoptosis or other cell death pathway. The exemplary kill switches provided for herein are non-limiting and other types can also be used, such as those provided in U.S. Pat. Nos. 9,089,520; 9,393,292; 9,434,935; 9,913,882; 9,932,572; 10,525,110; 11,077,176; U.S. Publication Nos. 20200230216; 20210220403; 20210106622; and 20220125837; or PCT Publication Nos. WO2013040371; WO2014197638; WO2017106185; and WO2020076738, all of which are hereby incorporated by reference in their entirety. In some embodiments, a cell comprises the pro-apoptotic polypeptide Caspase 9, which can be referred to as a kill-switch.

In some embodiments, engineering a cell to comprise a small molecule inducible kill switch may comprise a base epithelial cell line and a vector system. In some embodiments, the vector system may comprise a highly modular PiggyBAC transposon system. In some embodiments, the vector system may comprise a strong promotor that is constitutively expressed, a kozak translation initiation sequence, the sequence of a protein of interest (e.g., cytokine, chemokine, or other protein), an internal ribosome entry site, a kill switch sequence, a puromycin resistance sequence, and a poly-A tail between two inverted terminal repeat sequences.

In some embodiments, the cell is engineered to express an inducible kill switch. In some embodiments, the inducible kill switch is Caspase 9, which dimerizes or multimerizes upon binding of AP1903 (Rimiducid; [(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl](2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxyphenyl]propyl](2S)-1-[(2S)-2-(3,4,5-trimethoxyphenyl) butanoyl]piperidine-2-carboxylate).

In some embodiments, the cell may be engineered to produce or secrete a cytokine and to express an inducible kill switch. In some embodiments, the cytokine is mIL-2. In some embodiments, the mIL-2 and the inducible kill switch are encoded by the nucleic acid sequence of:

(SEQ ID NO: 22)
ATGTACAGCATGCAGCTCGCATCCTGTGTCACATTGACACTTGTGCTCCT
TGTCAACAGCGCACCCACTTCAAGCTCCACTTCAAGCTCTACAGCGGAAG
CACAGCAGCAGCAGCAGCAGCAGCAGCAGCAGCAGCAGCACCTGGAGCAG
CTGTTGATGGACCTACAGGAGCTCCTGAGCAGGATGGAGAATTACAGGAA
CCTGAAACTCCCCAGGATGCTCACCTTCAAATTTTACTTGCCCAAGCAGG
CCACAGAATTGAAAGATCTTCAGTGCCTAGAAGATGAACTTGGACCTCTG
CGGCATGTTCTGGATTTGACTCAAAGCAAAAGCTTTCAATTGGAAGATGC
TGAGAATTTCATCAGCAATATCAGAGTAACTGTTGTAAAACTAAAGGGCT
CTGACAACACATTTGAGTGCCAATTCGATGATGAGTCAGCAACTGTGGTG
GACTTTCTGAGGAGATGGATAGCCTTCTGTCAAAGCATCATCTCAACAAG
CCCTCAATAAGCCCCTCTCCCTCCCCCCCCCCTAACGTTACTGGCCGAAG
CCGCTTGGAATAAGGCCGGTGTGCGTTTGTCTATATGTTATTTTCCACCA
TATTGCCGTCTTTTGGCAATGTGAGGGCCCGGAAACCTGGCCCTGTCTTC
TTGACGAGCATTCCTAGGGGTCTTTCCCCTCTCGCCAAAGGAATGCAAGG
TCTGTTGAATGTCGTGAAGGAAGCAGTTCCTCTGGAAGCTTCTTGAAGAC
AAACAACGTCTGTAGCGACCCTTTGCAGGCAGCGGAACCCCCCACCTGGC
GACAGGTGCCTCTGCGGCCAAAAGCCACGTGTATAAGATACACCTGCAAA
GGCGGCACAACCCCAGTGCCACGTTGTGAGTTGGATAGTTGTGGAAAGAG
TCAAATGGCTCTCCTCAAGCGTATTCAACAAGGGGCTGAAGGATGCCCAG
AAGGTACCCCATTGTATGGGATCTGATCTGGGGCCTCGGTGCACATGCTT
TACATGTGTTTAGTCGAGGTTAAAAAAACGTCTAGGCCCCCCGAACCACG
GGGACGTGGTTTTCCTTTGAAAAACACGATGATAATATGGCCACAACCAT
GCTCGAGGGAGTGCAGGTGGAGACTATCTCCCCAGGAGACGGGCGCACCT
TCCCCAAGCGCGGCCAGACCTGCGTGGTGCACTACACCGGGATGCTTGAA
GATGGAAAGAAAGTTGATTCCTCCCGGGACAGAAACAAGCCCTTTAAGTT
TATGCTAGGCAAGCAGGAGGTGATCCGAGGCTGGGAAGAAGGGGTTGCCC
AGATGAGTGTGGGTCAGAGAGCCAAACTGACTATATCTCCAGATTATGCC
TATGGTGCCACTGGGCACCCAGGCATCATCCCACCACATGCCACTCTCGT
CTTCGATGTGGAGCTTCTAAAACTGGAATCTGGCGGTGGATCCGGAGTCG
ACGGATTTGGTGATGTCGGTGCTCTTGAGAGTTTGAGGGGAAATGCAGAT
TTGGCTTACATCCTGAGCATGGAGCCCTGTGGCCACTGCCTCATTATCAA
CAATGTGAACTTCTGCCGTGAGTCCGGGCTCCGCACCCGCACTGGCTCCA
ACATCGACTGTGAGAAGTTGCGGCGTCGCTTCTCCTCGCTGCATTTCATG
GTGGAGGTGAAGGGCGACCTGACTGCCAAGAAAATGGTGCTGGCTTTGCT
GGAGCTGGCGCGGCAGGACCACGGTGCTCTGGACTGCTGCGTGGTGGTCA
TTCTCTCTCACGGCTGTCAGGCCAGCCACCTGCAGTTCCCAGGGGCTGTC
TACGGCACAGATGGATGCCCTGTGTCGGTCGAGAAGATTGTGAACATCTT
CAATGGGACCAGCTGCCCCAGCCTGGGAGGGAAGCCCAAGCTCTTTTTCA
TCCAGGCCTGTGGTGGGGAGCAGAAAGACCATGGGTTTGAGGTGGCCTCC
ACTTCCCCTGAAGACGAGTCCCCTGGCAGTAACCCCGAGCCAGATGCCAC
CCCGTTCCAGGAAGGTTTGAGGACCTTCGACCAGCTGGACGCCATATCTA
GTTTGCCCACACCCAGTGACATCTTTGTGTCCTACTCTACTTTCCCAGGT
TTTGTTTCCTGGAGGGACCCCAAGAGTGGCTCCTGGTACGTTGAGACCCT
GGACGACATCTTTGAGCAGTGGGCTCACTCTGAAGACCTGCAGTCCCTCC
TGCTTAGGGTCGCTAATGCTGTTTCGGTGAAAGGGATTTATAAACAGATG
CCTGGTTGCTTTAATTTCCTCCGGAAAAAACTTTTCTTTAAAACATCAGT
CGACTATCCGTACGACGTACCAGACTACGCACTCGACTAA

In some embodiments, the cytokine is mIL-12. In some embodiments, the mIL-12 and the inducible kill switch are encoded by the nucleic acid sequence of:

(SEQ ID NO: 23)
ATGTGTCAATCACGCTACCTCCTCTTTTTGGCCACCCTTGCCCTCCTAAAC
CACCTCAGTTTGGCCAGGGTCATTCCAGTCTCTGGACCTGCCAGGTGTCT
TAGCCAGTCCCGAAACCTGCTGAAGACCACAGATGACATGGTGAAGACGG
CCAGAGAAAAACTGAAACATTATTCCTGCACTGCTGAAGACATCGATCAT
GAAGACATCACACGGGACCAAACCAGCACATTGAAGACCTGTTTACCACT
GGAACTACACAAGAACGAGAGTTGCCTGGCTACTAGAGAGACTTCTTCCA
CAACAAGAGGGAGCTGCCTGCCCCCACAGAAGACGTCTTTGATGATGACC
CTGTGCCTTGGTAGCATCTATGAGGACTTGAAGATGTACCAGACAGAGTT
CCAGGCCATCAACGCAGCACTTCAGAATCACAACCATCAGCAGATCATTC
TAGACAAGGGCATGCTGGTGGCCATCGATGAGCTGATGCAGTCTCTGAAT
CATAATGGCGAGACTCTGCGCCAGAAACCTCCTGTGGGAGAAGCAGACCC
TTACAGAGTGAAAATGAAGCTCTGCATCCTGCTTCACGCCTTCAGCACCC
GCGTCGTGACCATCAACAGGGTGATGGGCTATCTGAGCTCCGCCGGAAGC
GGAGAGGGCAGGGGAAGTCTTCTAACATGCGGGGACGTGGAGGAAAATCC
CGGCCCCATGTGTCCTCAGAAGCTAACCATCTCCTGGTTTGCCATCGTTT
TGCTGGTGTCTCCACTCATGGCCATGTGGGAGCTGGAGAAAGACGTTTAT
GTTGTAGAGGTGGACTGGACTCCCGATGCCCCTGGAGAAACAGTGAACCT
CACCTGTGACACGCCTGAAGAAGATGACATCACCTGGACCTCAGACCAGA
GACATGGAGTCATAGGCTCTGGAAAGACCCTGACCATCACTGTCAAAGAG
TTTCTAGATGCTGGCCAGTACACCTGCCACAAAGGAGGCGAGACTCTGAG
CCACTCACATCTGCTGCTCCACAAGAAGGAAAATGGAATTTGGTCCACTG
AAATTTTAAAAAATTTCAAAAACAAGACTTTCCTGAAGTGTGAAGCACCA
AATTACTCCGGACGGTTCACGTGCTCATGGCTGGTGCAAAGAAACATGGA
CTTGAAGTTCAACATCAAGAGCAGTAGCAGTTCCCCTGACTCTCGGGCAG
TGACATGTGGAATGGCGTCTCTGTCTGCAGAGAAGGTCACACTGGACCAA
AGGGACTATGAGAAGTATTCAGTGTCCTGCCAGGAGGATGTCACCTGCCC
AACTGCCGAGGAGACCCTGCCCATTGAACTGGCGTTGGAAGCACGGCAGC
AGAATAAATATGAGAACTACAGCACCAGCTTCTTCATCAGGGACATCATC
AAACCAGACCCGCCCAAGAACTTGCAGATGAAGCCTTTGAAGAACTCACA
GGTGGAGGTCAGCTGGGAGTACCCTGACTCCTGGAGCACTCCCCATTCCT
ACTTCTCCCTCAAGTTCTTTGTTCGAATCCAGCGCAAGAAAGAAAAGATG
AAGGAGACAGAGGAGGGGTGTAACCAGAAAGGTGCGTTCCTCGTAGAGAA
GACATCTACCGAAGTCCAATGCAAAGGCGGGAATGTCTGCGTGCAAGCTC
AGGATCGCTATTACAATTCCTCATGCAGCAAGTGGGCATGTGTTCCCTGC
AGGGTCCGATCCTAGACCCAGCTTTCTTGTACAAAGTGGGCCCCTCTCCC
TCCCCCCCCCCTAACGTTACTGGCCGAAGCCGCTTGGAATAAGGCCGGTG
TGCGTTTGTCTATATGTTATTTTCCACCATATTGCCGTCTTTTGGCAATG
TGAGGGCCCGGAAACCTGGCCCTGTCTTCTTGACGAGCATTCCTAGGGGT
CTTTCCCCTCTCGCCAAAGGAATGCAAGGTCTGTTGAATGTCGTGAAGGA
AGCAGTTCCTCTGGAAGCTTCTTGAAGACAAACAACGTCTGTAGCGACCC
TTTGCAGGCAGCGGAACCCCCCACCTGGCGACAGGTGCCTCTGCGGCCAA
AAGCCACGTGTATAAGATACACCTGCAAAGGCGGCACAACCCCAGTGCCA
CGTTGTGAGTTGGATAGTTGTGGAAAGAGTCAAATGGCTCTCCTCAAGCG
TATTCAACAAGGGGCTGAAGGATGCCCAGAAGGTACCCCATTGTATGGGA
TCTGATCTGGGGCCTCGGTGCACATGCTTTACATGTGTTTAGTCGAGGTT
AAAAAAACGTCTAGGCCCCCCGAACCACGGGGACGTGGTTTTCCTTTGAA
AAACACGATGATAATATGGCCACAACCATGCTCGAGGGAGTGCAGGTGGA
GACTATCTCCCCAGGAGACGGGCGCACCTTCCCCAAGCGCGGCCAGACCT
GCGTGGTGCACTACACCGGGATGCTTGAAGATGGAAAGAAAGTTGATTCC
TCCCGGGACAGAAACAAGCCCTTTAAGTTTATGCTAGGCAAGCAGGAGGT
GATCCGAGGCTGGGAAGAAGGGGTTGCCCAGATGAGTGTGGGTCAGAGAG
CCAAACTGACTATATCTCCAGATTATGCCTATGGTGCCACTGGGCACCCA
GGCATCATCCCACCACATGCCACTCTCGTCTTCGATGTGGAGCTTCTAAA
ACTGGAATCTGGCGGTGGATCCGGAGTCGACGGATTTGGTGATGTCGGTG
CTCTTGAGAGTTTGAGGGGAAATGCAGATTTGGCTTACATCCTGAGCATG
GAGCCCTGTGGCCACTGCCTCATTATCAACAATGTGAACTTCTGCCGTGA
GTCCGGGCTCCGCACCCGCACTGGCTCCAACATCGACTGTGAGAAGTTGC
GGCGTCGCTTCTCCTCGCTGCATTTCATGGTGGAGGTGAAGGGCGACCTG
ACTGCCAAGAAAATGGTGCTGGCTTTGCTGGAGCTGGCGCGGCAGGACCA
CGGTGCTCTGGACTGCTGCGTGGTGGTCATTCTCTCTCACGGCTGTCAGG
CCAGCCACCTGCAGTTCCCAGGGGCTGTCTACGGCACAGATGGATGCCCT
GTGTCGGTCGAGAAGATTGTGAACATCTTCAATGGGACCAGCTGCCCCAG
CCTGGGAGGGAAGCCCAAGCTCTTTTTCATCCAGGCCTGTGGTGGGGAGC
AGAAAGACCATGGGTTTGAGGTGGCCTCCACTTCCCCTGAAGACGAGTCC
CCTGGCAGTAACCCCGAGCCAGATGCCACCCCGTTCCAGGAAGGTTTGAG
GACCTTCGACCAGCTGGACGCCATATCTAGTTTGCCCACACCCAGTGACA
TCTTTGTGTCCTACTCTACTTTCCCAGGTTTTGTTTCCTGGAGGGACCCC
AAGAGTGGCTCCTGGTACGTTGAGACCCTGGACGACATCTTTGAGCAGTG
GGCTCACTCTGAAGACCTGCAGTCCCTCCTGCTTAGGGTCGCTAATGCTG
TTTCGGTGAAAGGGATTTATAAACAGATGCCTGGTTGCTTTAATTTCCTC
CGGAAAAAACTTTTCTTTAAAACATCAGTCGACTATCCGTACGACGTACC
AGACTACGCACTCGACTAA

In some embodiments, a composition comprises two or more populations of encapsulated cells: wherein a first population of encapsulated cells comprises an inducible kill switch and a first heterologous nucleic acid molecule encoding a first cytokine; and a second population of encapsulated cells comprises an inducible kill switch and a second heterologous nucleic acid molecule encoding a second cytokine. In some embodiments, the composition comprises two or more populations of encapsulated cells: wherein a first population of encapsulated cells comprises an inducible kill switch and a first heterologous nucleic acid molecule encoding an IL-12; and a second population of encapsulated cells comprises an inducible kill switch and a second heterologous nucleic acid molecule encoding a second cytokine. In some embodiments, the composition comprises two or more populations of encapsulated cells: wherein a first population of encapsulated cells comprises an inducible kill switch and a first heterologous nucleic acid molecule encoding an IL-2; and a second population of encapsulated cells comprises an inducible kill switch and a second heterologous nucleic acid molecule encoding a second cytokine. In some embodiments, the composition comprises two or more populations of encapsulated cells: wherein a first population of encapsulated cells comprises an inducible kill switch and a first heterologous nucleic acid molecule encoding an IL-15; and a second population of encapsulated cells comprises an inducible kill switch and a second heterologous nucleic acid molecule encoding a second cytokine. In some embodiments, the composition comprises two or more populations of encapsulated cells: wherein a first population of encapsulated cells comprises an inducible kill switch and a first heterologous nucleic acid molecule encoding an IFN-α; and a second population of encapsulated cells comprises an inducible kill switch and a second heterologous nucleic acid molecule encoding a second cytokine. In some embodiments, the composition comprises two or more populations of encapsulated cells: wherein a first population of encapsulated cells comprises an inducible kill switch and a first heterologous nucleic acid molecule encoding a IFN-γ; and a second population of encapsulated cells comprises an inducible kill switch and a second heterologous nucleic acid molecule encoding a second cytokine.

In some embodiments, a composition comprises two or more populations of encapsulated cells: wherein a first population of encapsulated cells comprises an inducible kill switch and a first heterologous nucleic acid molecule encoding a first cytokine; and a second population of encapsulated cells comprises an inducible kill switch and a second heterologous nucleic acid molecule encoding an antibody that binds to a checkpoint inhibitor. In some embodiments, the composition comprises two or more populations of encapsulated cells: wherein a first population of encapsulated cells comprises an inducible kill switch and a first heterologous nucleic acid molecule encoding an IL-12; and a second population of encapsulated cells comprises an inducible kill switch and a second heterologous nucleic acid molecule encoding an antibody that binds to a checkpoint inhibitor. In some embodiments, the composition comprises two or more populations of encapsulated cells: wherein a first population of encapsulated cells comprises an inducible kill switch and a first heterologous nucleic acid molecule encoding an IL-2; and a second population of encapsulated cells comprises an inducible kill switch and a second heterologous nucleic acid molecule encoding an antibody that binds to a checkpoint inhibitor. In some embodiments, the composition comprises two or more populations of encapsulated cells: wherein a first population of encapsulated cells comprises an inducible kill switch and a first heterologous nucleic acid molecule encoding an IL-15; and a second population of encapsulated cells comprises an inducible kill switch and a second heterologous nucleic acid molecule encoding an antibody that binds to a checkpoint inhibitor. In some embodiments, the composition comprises two or more populations of encapsulated cells: wherein a first population of encapsulated cells comprises an inducible kill switch and a first heterologous nucleic acid molecule encoding an IFN-α; and a second population of encapsulated cells comprises an inducible kill switch and a second heterologous nucleic acid molecule encoding an antibody that binds to a checkpoint inhibitor. In some embodiments, the composition comprises two or more populations of encapsulated cells: wherein a first population of encapsulated cells comprises an inducible kill switch and a first heterologous nucleic acid molecule encoding a IFN-γ; and a second population of encapsulated cells comprises an inducible kill switch and a second heterologous nucleic acid molecule encoding an antibody that binds to a checkpoint inhibitor.

In some embodiments, the first population of encapsulated cells comprising an inducible kill switch and the first heterologous nucleic acid molecule encoding the first cytokine, comprises Caspase 9, and the first heterologous nucleic acid molecule encoding IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN—B, IFN-γ, TNF-α, TNF-b, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, TRAIL, SCF, M-CSF, MSP (membrane scaffold protein), 4-1BBL, LIF (Leukemia Inhibitory Factor protein), or OSM (Oncostatin M). In some embodiments, the first population of encapsulated cells comprising an inducible kill switch and the first heterologous nucleic acid molecule encoding the first cytokine, comprises Caspase 9, and the first heterologous nucleic acid molecule encoding IL-1. In some embodiments, the first population of encapsulated cells comprising an inducible kill switch and the first heterologous nucleic acid molecule encoding the first cytokine, comprises Caspase 9, and the first heterologous nucleic acid molecule encoding IL-1a. In some embodiments, the first population of encapsulated cells comprising an inducible kill switch and the first heterologous nucleic acid molecule encoding the first cytokine, comprises Caspase 9, and the first heterologous nucleic acid molecule encoding IL-1b. In some embodiments, the first population of encapsulated cells comprising an inducible kill switch and the first heterologous nucleic acid molecule encoding the first cytokine, comprises Caspase 9, and the first heterologous nucleic acid molecule encoding IL-1RA. In some embodiments, the first population of encapsulated cells comprising an inducible kill switch and the first heterologous nucleic acid molecule encoding the first cytokine, comprises Caspase 9, and the first heterologous nucleic acid molecule encoding IL-2. In some embodiments, the first population of encapsulated cells comprising an inducible kill switch and the first heterologous nucleic acid molecule encoding the first cytokine, comprises Caspase 9, and the first heterologous nucleic acid molecule encoding IL-4. In some embodiments, the first population of encapsulated cells comprising an inducible kill switch and the first heterologous nucleic acid molecule encoding the first cytokine, comprises Caspase 9, and the first heterologous nucleic acid molecule encoding IL-5. In some embodiments, the first population of encapsulated cells comprising an inducible kill switch and the first heterologous nucleic acid molecule encoding the first cytokine, comprises Caspase 9, and the first heterologous nucleic acid molecule encoding IL-6. In some embodiments, the first population of encapsulated cells comprising an inducible kill switch and the first heterologous nucleic acid molecule encoding the first cytokine, comprises Caspase 9, and the first heterologous nucleic acid molecule encoding IL-7. In some embodiments, the first population of encapsulated cells comprising an inducible kill switch and the first heterologous nucleic acid molecule encoding the first cytokine, comprises Caspase 9, and the first heterologous nucleic acid molecule encoding IL-8. In some embodiments, the first population of encapsulated cells comprising an inducible kill switch and the first heterologous nucleic acid molecule encoding the first cytokine, comprises Caspase 9, and the first heterologous nucleic acid molecule encoding IL-9. In some embodiments, the first population of encapsulated cells comprising an inducible kill switch and the first heterologous nucleic acid molecule encoding the first cytokine, comprises Caspase 9, and the first heterologous nucleic acid molecule encoding IL-10. In some embodiments, the first population of encapsulated cells comprising an inducible kill switch and the first heterologous nucleic acid molecule encoding the first cytokine, comprises Caspase 9, and the first heterologous nucleic acid molecule encoding IL-11. In some embodiments, the first population of encapsulated cells comprising an inducible kill switch and the first heterologous nucleic acid molecule encoding the first cytokine, comprises Caspase 9, and the first heterologous nucleic acid molecule encoding IL-12. In some embodiments, the first population of encapsulated cells comprising an inducible kill switch and the first heterologous nucleic acid molecule encoding the first cytokine, comprises Caspase 9, and the first heterologous nucleic acid molecule encoding IL-12a. In some embodiments, the first population of encapsulated cells comprising an inducible kill switch and the first heterologous nucleic acid molecule encoding the first cytokine, comprises Caspase 9, and the first heterologous nucleic acid molecule encoding IL-12b. In some embodiments, the first population of encapsulated cells comprising an inducible kill switch and the first heterologous nucleic acid molecule encoding the first cytokine, comprises Caspase 9, and the first heterologous nucleic acid molecule encoding IL-13. In some embodiments, the first population of encapsulated cells comprising an inducible kill switch and the first heterologous nucleic acid molecule encoding the first cytokine, comprises Caspase 9, and the first heterologous nucleic acid molecule encoding IL-14. In some embodiments, the first population of encapsulated cells comprising an inducible kill switch and the first heterologous nucleic acid molecule encoding the first cytokine, comprises Caspase 9, and the first heterologous nucleic acid molecule encoding IL-15. In some embodiments, the first population of encapsulated cells comprising an inducible kill switch and the first heterologous nucleic acid molecule encoding the first cytokine, comprises Caspase 9, and the first heterologous nucleic acid molecule encoding IL-16. In some embodiments, the first population of encapsulated cells comprising an inducible kill switch and the first heterologous nucleic acid molecule encoding the first cytokine, comprises Caspase 9, and the first heterologous nucleic acid molecule encoding IL-17. In some embodiments, the first population of encapsulated cells comprising an inducible kill switch and the first heterologous nucleic acid molecule encoding the first cytokine, comprises Caspase 9, and the first heterologous nucleic acid molecule encoding IL-20. In some embodiments, the first population of encapsulated cells comprising an inducible kill switch and the first heterologous nucleic acid molecule encoding the first cytokine, comprises Caspase 9, and the first heterologous nucleic acid molecule encoding IFN-α. In some embodiments, the first population of encapsulated cells comprising an inducible kill switch and the first heterologous nucleic acid molecule encoding the first cytokine, comprises Caspase 9, and the first heterologous nucleic acid molecule encoding IFN-β. In some embodiments, the first population of encapsulated cells comprising an inducible kill switch and the first heterologous nucleic acid molecule encoding the first cytokine, comprises Caspase 9, and the first heterologous nucleic acid molecule encoding IFN-γ. In some embodiments, the first population of encapsulated cells comprising an inducible kill switch and the first heterologous nucleic acid molecule encoding the first cytokine, comprises Caspase 9, and the first heterologous nucleic acid molecule encoding TNF-α. In some embodiments, the first population of encapsulated cells comprising an inducible kill switch and the first heterologous nucleic acid molecule encoding the first cytokine, comprises Caspase 9, and the first heterologous nucleic acid molecule encoding TNF-b. In some embodiments, the first population of encapsulated cells comprising an inducible kill switch and the first heterologous nucleic acid molecule encoding the first cytokine, comprises Caspase 9, and the first heterologous nucleic acid molecule encoding TGF-b. In some embodiments, the first population of encapsulated cells comprising an inducible kill switch and the first heterologous nucleic acid molecule encoding the first cytokine, comprises Caspase 9, and the first heterologous nucleic acid molecule encoding CCL-1. In some embodiments, the first population of encapsulated cells comprising an inducible kill switch and the first heterologous nucleic acid molecule encoding the first cytokine, comprises Caspase 9, and the first heterologous nucleic acid molecule encoding CCL-2. In some embodiments, the first population of encapsulated cells comprising an inducible kill switch and the first heterologous nucleic acid molecule encoding the first cytokine, comprises Caspase 9, and the first heterologous nucleic acid molecule encoding CCL-3. In some embodiments, the first population of encapsulated cells comprising an inducible kill switch and the first heterologous nucleic acid molecule encoding the first cytokine, comprises Caspase 9, and the first heterologous nucleic acid molecule encoding CCL-4. In some embodiments, the first population of encapsulated cells comprising an inducible kill switch and the first heterologous nucleic acid molecule encoding the first cytokine, comprises Caspase 9, and the first heterologous nucleic acid molecule encoding CCL-5. In some embodiments, the first population of encapsulated cells comprising an inducible kill switch and the first heterologous nucleic acid molecule encoding the first cytokine, comprises Caspase 9, and the first heterologous nucleic acid molecule encoding CCL-6. In some embodiments, the first population of encapsulated cells comprising an inducible kill switch and the first heterologous nucleic acid molecule encoding the first cytokine, comprises Caspase 9, and the first heterologous nucleic acid molecule encoding CCL-7. In some embodiments, the first population of encapsulated cells comprising an inducible kill switch and the first heterologous nucleic acid molecule encoding the first cytokine, comprises Caspase 9, and the first heterologous nucleic acid molecule encoding CCL-8. In some embodiments, the first population of encapsulated cells comprising an inducible kill switch and the first heterologous nucleic acid molecule encoding the first cytokine, comprises Caspase 9, and the first heterologous nucleic acid molecule encoding CCL-9. In some embodiments, the first population of encapsulated cells comprising an inducible kill switch and the first heterologous nucleic acid molecule encoding the first cytokine, comprises Caspase 9, and the first heterologous nucleic acid molecule encoding CCL-10. In some embodiments, the first population of encapsulated cells comprising an inducible kill switch and the first heterologous nucleic acid molecule encoding the first cytokine, comprises Caspase 9, and the first heterologous nucleic acid molecule encoding CCL-11. In some embodiments, the first population of encapsulated cells comprising an inducible kill switch and the first heterologous nucleic acid molecule encoding the first cytokine, comprises Caspase 9, and the first heterologous nucleic acid molecule encoding CCL-12. In some embodiments, the first population of encapsulated cells comprising an inducible kill switch and the first heterologous nucleic acid molecule encoding the first cytokine, comprises Caspase 9, and the first heterologous nucleic acid molecule encoding CCL-13. In some embodiments, the first population of encapsulated cells comprising an inducible kill switch and the first heterologous nucleic acid molecule encoding the first cytokine, comprises Caspase 9, and the first heterologous nucleic acid molecule encoding CCL-14. In some embodiments, the first population of encapsulated cells comprising an inducible kill switch and the first heterologous nucleic acid molecule encoding the first cytokine, comprises Caspase 9, and the first heterologous nucleic acid molecule encoding CCL-15. In some embodiments, the first population of encapsulated cells comprising an inducible kill switch and the first heterologous nucleic acid molecule encoding the first cytokine, comprises Caspase 9, and the first heterologous nucleic acid molecule encoding CCL-16. In some embodiments, the first population of encapsulated cells comprising an inducible kill switch and the first heterologous nucleic acid molecule encoding the first cytokine, comprises Caspase 9, and the first heterologous nucleic acid molecule encoding CCL-17. In some embodiments, the first population of encapsulated cells comprising an inducible kill switch and the first heterologous nucleic acid molecule encoding the first cytokine, comprises Caspase 9, and the first heterologous nucleic acid molecule encoding CCL-18. In some embodiments, the first population of encapsulated cells comprising an inducible kill switch and the first heterologous nucleic acid molecule encoding the first cytokine, comprises Caspase 9, and the first heterologous nucleic acid molecule encoding CCL-19. In some embodiments, the first population of encapsulated cells comprising an inducible kill switch and the first heterologous nucleic acid molecule encoding the first cytokine, comprises Caspase 9, and the first heterologous nucleic acid molecule encoding CCL-20. In some embodiments, the first population of encapsulated cells comprising an inducible kill switch and the first heterologous nucleic acid molecule encoding the first cytokine, comprises Caspase 9, and the first heterologous nucleic acid molecule encoding CCL-21. In some embodiments, the first population of encapsulated cells comprising an inducible kill switch and the first heterologous nucleic acid molecule encoding the first cytokine, comprises Caspase 9, and the first heterologous nucleic acid molecule encoding CCL-22. In some embodiments, the first population of encapsulated cells comprising an inducible kill switch and the first heterologous nucleic acid molecule encoding the first cytokine, comprises Caspase 9, and the first heterologous nucleic acid molecule encoding CCL-23. In some embodiments, the first population of encapsulated cells comprising an inducible kill switch and the first heterologous nucleic acid molecule encoding the first cytokine, comprises Caspase 9, and the first heterologous nucleic acid molecule encoding CCL-24. In some embodiments, the first population of encapsulated cells comprising an inducible kill switch and the first heterologous nucleic acid molecule encoding the first cytokine, comprises Caspase 9, and the first heterologous nucleic acid molecule encoding CCL-25. In some embodiments, the first population of encapsulated cells comprising an inducible kill switch and the first heterologous nucleic acid molecule encoding the first cytokine, comprises Caspase 9, and the first heterologous nucleic acid molecule encoding CCL-26. In some embodiments, the first population of encapsulated cells comprising an inducible kill switch and the first heterologous nucleic acid molecule encoding the first cytokine, comprises Caspase 9, and the first heterologous nucleic acid molecule encoding CCL-27. In some embodiments, the first population of encapsulated cells comprising an inducible kill switch and the first heterologous nucleic acid molecule encoding the first cytokine, comprises Caspase 9, and the first heterologous nucleic acid molecule encoding CCL-28. In some embodiments, the first population of encapsulated cells comprising an inducible kill switch and the first heterologous nucleic acid molecule encoding the first cytokine, comprises Caspase 9, and the first heterologous nucleic acid molecule encoding CXCL1. In some embodiments, the first population of encapsulated cells comprising an inducible kill switch and the first heterologous nucleic acid molecule encoding the first cytokine, comprises Caspase 9, and the first heterologous nucleic acid molecule encoding CXCL2. In some embodiments, the first population of encapsulated cells comprising an inducible kill switch and the first heterologous nucleic acid molecule encoding the first cytokine, comprises Caspase 9, and the first heterologous nucleic acid molecule encoding CXCL3. In some embodiments, the first population of encapsulated cells comprising an inducible kill switch and the first heterologous nucleic acid molecule encoding the first cytokine, comprises Caspase 9, and the first heterologous nucleic acid molecule encoding CXCL5. In some embodiments, the first population of encapsulated cells comprising an inducible kill switch and the first heterologous nucleic acid molecule encoding the first cytokine, comprises Caspase 9, and the first heterologous nucleic acid molecule encoding CXCL6. In some embodiments, the first population of encapsulated cells comprising an inducible kill switch and the first heterologous nucleic acid molecule encoding the first cytokine, comprises Caspase 9, and the first heterologous nucleic acid molecule encoding CXCL7. In some embodiments, the first population of encapsulated cells comprising an inducible kill switch and the first heterologous nucleic acid molecule encoding the first cytokine, comprises Caspase 9, and the first heterologous nucleic acid molecule encoding CXCL8. In some embodiments, the first population of encapsulated cells comprising an inducible kill switch and the first heterologous nucleic acid molecule encoding the first cytokine, comprises Caspase 9, and the first heterologous nucleic acid molecule encoding CXCL9. In some embodiments, the first population of encapsulated cells comprising an inducible kill switch and the first heterologous nucleic acid molecule encoding the first cytokine, comprises Caspase 9, and the first heterologous nucleic acid molecule encoding CXCL10. In some embodiments, the first population of encapsulated cells comprising an inducible kill switch and the first heterologous nucleic acid molecule encoding the first cytokine, comprises Caspase 9, and the first heterologous nucleic acid molecule encoding CXCL11. In some embodiments, the first population of encapsulated cells comprising an inducible kill switch and the first heterologous nucleic acid molecule encoding the first cytokine, comprises Caspase 9, and the first heterologous nucleic acid molecule encoding CXCL12. In some embodiments, the first population of encapsulated cells comprising an inducible kill switch and the first heterologous nucleic acid molecule encoding the first cytokine, comprises Caspase 9, and the first heterologous nucleic acid molecule encoding CXCL13. In some embodiments, the first population of encapsulated cells comprising an inducible kill switch and the first heterologous nucleic acid molecule encoding the first cytokine, comprises Caspase 9, and the first heterologous nucleic acid molecule encoding CXCL14. In some embodiments, the first population of encapsulated cells comprising an inducible kill switch and the first heterologous nucleic acid molecule encoding the first cytokine, comprises Caspase 9, and the first heterologous nucleic acid molecule encoding CXCL15. In some embodiments, the first population of encapsulated cells comprising an inducible kill switch and the first heterologous nucleic acid molecule encoding the first cytokine, comprises Caspase 9, and the first heterologous nucleic acid molecule encoding CXCL16. In some embodiments, the first population of encapsulated cells comprising an inducible kill switch and the first heterologous nucleic acid molecule encoding the first cytokine, comprises Caspase 9, and the first heterologous nucleic acid molecule encoding CXCL17. In some embodiments, the first population of encapsulated cells comprising an inducible kill switch and the first heterologous nucleic acid molecule encoding the first cytokine, comprises Caspase 9, and the first heterologous nucleic acid molecule encoding TRAIL. In some embodiments, the first population of encapsulated cells comprising an inducible kill switch and the first heterologous nucleic acid molecule encoding the first cytokine, comprises Caspase 9, and the first heterologous nucleic acid molecule encoding SCF. In some embodiments, the first population of encapsulated cells comprising an inducible kill switch and the first heterologous nucleic acid molecule encoding the first cytokine, comprises Caspase 9, and the first heterologous nucleic acid molecule encoding M-CSF. In some embodiments, the first population of encapsulated cells comprising an inducible kill switch and the first heterologous nucleic acid molecule encoding the first cytokine, comprises Caspase 9, and the first heterologous nucleic acid molecule encoding MSP (membrane scaffold protein). In some embodiments, the first population of encapsulated cells comprising an inducible kill switch and the first heterologous nucleic acid molecule encoding the first cytokine, comprises Caspase 9, and the first heterologous nucleic acid molecule encoding 4-1BBL. In some embodiments, the first population of encapsulated cells comprising an inducible kill switch and the first heterologous nucleic acid molecule encoding the first cytokine, comprises Caspase 9, and the first heterologous nucleic acid molecule encoding LIF (Leukemia Inhibitory Factor protein). In some embodiments, the first population of encapsulated cells comprising an inducible kill switch and the first heterologous nucleic acid molecule encoding the first cytokine, comprises Caspase 9, and the first heterologous nucleic acid molecule encoding OSM (Oncostatin M).

In some embodiments, the second population of encapsulated cells comprising an inducible kill switch and the second heterologous nucleic acid molecule encoding the second cytokine, comprises Caspase 9, and the second heterologous nucleic acid molecule encoding IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-β, IFN-γ, TNF-a, TNF-b, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, TRAIL, SCF, M-CSF, MSP (membrane scaffold protein), 4-1BBL, LIF (Leukemia Inhibitory Factor protein), or OSM (Oncostatin M). In some embodiments, the second population of encapsulated cells comprising an inducible kill switch and the second heterologous nucleic acid molecule encoding the second cytokine, comprises Caspase 9, and the second heterologous nucleic acid molecule encoding IL-1. In some embodiments, the second population of encapsulated cells comprising an inducible kill switch and the second heterologous nucleic acid molecule encoding the second cytokine, comprises Caspase 9, and the second heterologous nucleic acid molecule encoding IL-1a. In some embodiments, the second population of encapsulated cells comprising an inducible kill switch and the second heterologous nucleic acid molecule encoding the second cytokine, comprises Caspase 9, and the second heterologous nucleic acid molecule encoding IL-1b. In some embodiments, the second population of encapsulated cells comprising an inducible kill switch and the second heterologous nucleic acid molecule encoding the second cytokine, comprises Caspase 9, and the second heterologous nucleic acid molecule encoding IL-1RA. In some embodiments, the second population of encapsulated cells comprising an inducible kill switch and the second heterologous nucleic acid molecule encoding the second cytokine, comprises Caspase 9, and the second heterologous nucleic acid molecule encoding IL-2. In some embodiments, the second population of encapsulated cells comprising an inducible kill switch and the second heterologous nucleic acid molecule encoding the second cytokine, comprises Caspase 9, and the second heterologous nucleic acid molecule encoding IL-4. In some embodiments, the second population of encapsulated cells comprising an inducible kill switch and the second heterologous nucleic acid molecule encoding the second cytokine, comprises Caspase 9, and the second heterologous nucleic acid molecule encoding IL-5. In some embodiments, the second population of encapsulated cells comprising an inducible kill switch and the second heterologous nucleic acid molecule encoding the second cytokine, comprises Caspase 9, and the second heterologous nucleic acid molecule encoding IL-6. In some embodiments, the second population of encapsulated cells comprising an inducible kill switch and the second heterologous nucleic acid molecule encoding the second cytokine, comprises Caspase 9, and the second heterologous nucleic acid molecule encoding IL-7. In some embodiments, the second population of encapsulated cells comprising an inducible kill switch and the second heterologous nucleic acid molecule encoding the second cytokine, comprises Caspase 9, and the second heterologous nucleic acid molecule encoding IL-8. In some embodiments, the second population of encapsulated cells comprising an inducible kill switch and the second heterologous nucleic acid molecule encoding the second cytokine, comprises Caspase 9, and the second heterologous nucleic acid molecule encoding IL-9. In some embodiments, the second population of encapsulated cells comprising an inducible kill switch and the second heterologous nucleic acid molecule encoding the second cytokine, comprises Caspase 9, and the second heterologous nucleic acid molecule encoding IL-10. In some embodiments, the second population of encapsulated cells comprising an inducible kill switch and the second heterologous nucleic acid molecule encoding the second cytokine, comprises Caspase 9, and the second heterologous nucleic acid molecule encoding IL-11. In some embodiments, the second population of encapsulated cells comprising an inducible kill switch and the second heterologous nucleic acid molecule encoding the second cytokine, comprises Caspase 9, and the second heterologous nucleic acid molecule encoding IL-12. In some embodiments, the second population of encapsulated cells comprising an inducible kill switch and the second heterologous nucleic acid molecule encoding the second cytokine, comprises Caspase 9, and the second heterologous nucleic acid molecule encoding IL-12a. In some embodiments, the second population of encapsulated cells comprising an inducible kill switch and the second heterologous nucleic acid molecule encoding the second cytokine, comprises Caspase 9, and the second heterologous nucleic acid molecule encoding IL-12b. In some embodiments, the second population of encapsulated cells comprising an inducible kill switch and the second heterologous nucleic acid molecule encoding the second cytokine, comprises Caspase 9, and the second heterologous nucleic acid molecule encoding IL-13. In some embodiments, the second population of encapsulated cells comprising an inducible kill switch and the second heterologous nucleic acid molecule encoding the second cytokine, comprises Caspase 9, and the second heterologous nucleic acid molecule encoding IL-14. In some embodiments, the second population of encapsulated cells comprising an inducible kill switch and the second heterologous nucleic acid molecule encoding the second cytokine, comprises Caspase 9, and the second heterologous nucleic acid molecule encoding IL-15. In some embodiments, the second population of encapsulated cells comprising an inducible kill switch and the second heterologous nucleic acid molecule encoding the second cytokine, comprises Caspase 9, and the second heterologous nucleic acid molecule encoding IL-16. In some embodiments, the second population of encapsulated cells comprising an inducible kill switch and the second heterologous nucleic acid molecule encoding the second cytokine, comprises Caspase 9, and the second heterologous nucleic acid molecule encoding IL-17. In some embodiments, the second population of encapsulated cells comprising an inducible kill switch and the second heterologous nucleic acid molecule encoding the second cytokine, comprises Caspase 9, and the second heterologous nucleic acid molecule encoding IL-20. In some embodiments, the second population of encapsulated cells comprising an inducible kill switch and the second heterologous nucleic acid molecule encoding the second cytokine, comprises Caspase 9, and the second heterologous nucleic acid molecule encoding IFN-α. In some embodiments, the second population of encapsulated cells comprising an inducible kill switch and the second heterologous nucleic acid molecule encoding the second cytokine, comprises Caspase 9, and the second heterologous nucleic acid molecule encoding IFN-β. In some embodiments, the second population of encapsulated cells comprising an inducible kill switch and the second heterologous nucleic acid molecule encoding the second cytokine, comprises Caspase 9, and the second heterologous nucleic acid molecule encoding IFN-γ. In some embodiments, the second population of encapsulated cells comprising an inducible kill switch and the second heterologous nucleic acid molecule encoding the second cytokine, comprises Caspase 9, and the second heterologous nucleic acid molecule encoding TNF-α. In some embodiments, the second population of encapsulated cells comprising an inducible kill switch and the second heterologous nucleic acid molecule encoding the second cytokine, comprises Caspase 9, and the second heterologous nucleic acid molecule encoding TNF-b. In some embodiments, the second population of encapsulated cells comprising an inducible kill switch and the second heterologous nucleic acid molecule encoding the second cytokine, comprises Caspase 9, and the second heterologous nucleic acid molecule encoding TGF-b. In some embodiments, the second population of encapsulated cells comprising an inducible kill switch and the second heterologous nucleic acid molecule encoding the second cytokine, comprises Caspase 9, and the second heterologous nucleic acid molecule encoding CCL-1. In some embodiments, the second population of encapsulated cells comprising an inducible kill switch and the second heterologous nucleic acid molecule encoding the second cytokine, comprises Caspase 9, and the second heterologous nucleic acid molecule encoding CCL-2. In some embodiments, the second population of encapsulated cells comprising an inducible kill switch and the second heterologous nucleic acid molecule encoding the second cytokine, comprises Caspase 9, and the second heterologous nucleic acid molecule encoding CCL-3. In some embodiments, the second population of encapsulated cells comprising an inducible kill switch and the second heterologous nucleic acid molecule encoding the second cytokine, comprises Caspase 9, and the second heterologous nucleic acid molecule encoding CCL-4. In some embodiments, the second population of encapsulated cells comprising an inducible kill switch and the second heterologous nucleic acid molecule encoding the second cytokine, comprises Caspase 9, and the second heterologous nucleic acid molecule encoding CCL-5. In some embodiments, the second population of encapsulated cells comprising an inducible kill switch and the second heterologous nucleic acid molecule encoding the second cytokine, comprises Caspase 9, and the second heterologous nucleic acid molecule encoding CCL-6. In some embodiments, the second population of encapsulated cells comprising an inducible kill switch and the second heterologous nucleic acid molecule encoding the second cytokine, comprises Caspase 9, and the second heterologous nucleic acid molecule encoding CCL-7. In some embodiments, the second population of encapsulated cells comprising an inducible kill switch and the second heterologous nucleic acid molecule encoding the second cytokine, comprises Caspase 9, and the second heterologous nucleic acid molecule encoding CCL-8. In some embodiments, the second population of encapsulated cells comprising an inducible kill switch and the second heterologous nucleic acid molecule encoding the second cytokine, comprises Caspase 9, and the second heterologous nucleic acid molecule encoding CCL-9. In some embodiments, the second population of encapsulated cells comprising an inducible kill switch and the second heterologous nucleic acid molecule encoding the second cytokine, comprises Caspase 9, and the second heterologous nucleic acid molecule encoding CCL-10. In some embodiments, the second population of encapsulated cells comprising an inducible kill switch and the second heterologous nucleic acid molecule encoding the second cytokine, comprises Caspase 9, and the second heterologous nucleic acid molecule encoding CCL-11. In some embodiments, the second population of encapsulated cells comprising an inducible kill switch and the second heterologous nucleic acid molecule encoding the second cytokine, comprises Caspase 9, and the second heterologous nucleic acid molecule encoding CCL-12. In some embodiments, the second population of encapsulated cells comprising an inducible kill switch and the second heterologous nucleic acid molecule encoding the second cytokine, comprises Caspase 9, and the second heterologous nucleic acid molecule encoding CCL-13. In some embodiments, the second population of encapsulated cells comprising an inducible kill switch and the second heterologous nucleic acid molecule encoding the second cytokine, comprises Caspase 9, and the second heterologous nucleic acid molecule encoding CCL-14. In some embodiments, the second population of encapsulated cells comprising an inducible kill switch and the second heterologous nucleic acid molecule encoding the second cytokine, comprises Caspase 9, and the second heterologous nucleic acid molecule encoding CCL-15. In some embodiments, the second population of encapsulated cells comprising an inducible kill switch and the second heterologous nucleic acid molecule encoding the second cytokine, comprises Caspase 9, and the second heterologous nucleic acid molecule encoding CCL-16. In some embodiments, the second population of encapsulated cells comprising an inducible kill switch and the second heterologous nucleic acid molecule encoding the second cytokine, comprises Caspase 9, and the second heterologous nucleic acid molecule encoding CCL-17. In some embodiments, the second population of encapsulated cells comprising an inducible kill switch and the second heterologous nucleic acid molecule encoding the second cytokine, comprises Caspase 9, and the second heterologous nucleic acid molecule encoding CCL-18. In some embodiments, the second population of encapsulated cells comprising an inducible kill switch and the second heterologous nucleic acid molecule encoding the second cytokine, comprises Caspase 9, and the second heterologous nucleic acid molecule encoding CCL-19. In some embodiments, the second population of encapsulated cells comprising an inducible kill switch and the second heterologous nucleic acid molecule encoding the second cytokine, comprises Caspase 9, and the second heterologous nucleic acid molecule encoding CCL-20. In some embodiments, the second population of encapsulated cells comprising an inducible kill switch and the second heterologous nucleic acid molecule encoding the second cytokine, comprises Caspase 9, and the second heterologous nucleic acid molecule encoding CCL-21. In some embodiments, the second population of encapsulated cells comprising an inducible kill switch and the second heterologous nucleic acid molecule encoding the second cytokine, comprises Caspase 9, and the second heterologous nucleic acid molecule encoding CCL-22. In some embodiments, the second population of encapsulated cells comprising an inducible kill switch and the second heterologous nucleic acid molecule encoding the second cytokine, comprises Caspase 9, and the second heterologous nucleic acid molecule encoding CCL-23. In some embodiments, the second population of encapsulated cells comprising an inducible kill switch and the second heterologous nucleic acid molecule encoding the second cytokine, comprises Caspase 9, and the second heterologous nucleic acid molecule encoding CCL-24. In some embodiments, the second population of encapsulated cells comprising an inducible kill switch and the second heterologous nucleic acid molecule encoding the second cytokine, comprises Caspase 9, and the second heterologous nucleic acid molecule encoding CCL-25. In some embodiments, the second population of encapsulated cells comprising an inducible kill switch and the second heterologous nucleic acid molecule encoding the second cytokine, comprises Caspase 9, and the second heterologous nucleic acid molecule encoding CCL-26. In some embodiments, the second population of encapsulated cells comprising an inducible kill switch and the second heterologous nucleic acid molecule encoding the second cytokine, comprises Caspase 9, and the second heterologous nucleic acid molecule encoding CCL-27. In some embodiments, the second population of encapsulated cells comprising an inducible kill switch and the second heterologous nucleic acid molecule encoding the second cytokine, comprises Caspase 9, and the second heterologous nucleic acid molecule encoding CCL-28. In some embodiments, the second population of encapsulated cells comprising an inducible kill switch and the second heterologous nucleic acid molecule encoding the second cytokine, comprises Caspase 9, and the second heterologous nucleic acid molecule encoding CXCL1. In some embodiments, the second population of encapsulated cells comprising an inducible kill switch and the second heterologous nucleic acid molecule encoding the second cytokine, comprises Caspase 9, and the second heterologous nucleic acid molecule encoding CXCL2. In some embodiments, the second population of encapsulated cells comprising an inducible kill switch and the second heterologous nucleic acid molecule encoding the second cytokine, comprises Caspase 9, and the second heterologous nucleic acid molecule encoding CXCL3. In some embodiments, the second population of encapsulated cells comprising an inducible kill switch and the second heterologous nucleic acid molecule encoding the second cytokine, comprises Caspase 9, and the second heterologous nucleic acid molecule encoding CXCL5. In some embodiments, the second population of encapsulated cells comprising an inducible kill switch and the second heterologous nucleic acid molecule encoding the second cytokine, comprises Caspase 9, and the second heterologous nucleic acid molecule encoding CXCL6. In some embodiments, the second population of encapsulated cells comprising an inducible kill switch and the second heterologous nucleic acid molecule encoding the second cytokine, comprises Caspase 9, and the second heterologous nucleic acid molecule encoding CXCL7. In some embodiments, the second population of encapsulated cells comprising an inducible kill switch and the second heterologous nucleic acid molecule encoding the second cytokine, comprises Caspase 9, and the second heterologous nucleic acid molecule encoding CXCL8. In some embodiments, the second population of encapsulated cells comprising an inducible kill switch and the second heterologous nucleic acid molecule encoding the second cytokine, comprises Caspase 9, and the second heterologous nucleic acid molecule encoding CXCL9. In some embodiments, the second population of encapsulated cells comprising an inducible kill switch and the second heterologous nucleic acid molecule encoding the second cytokine, comprises Caspase 9, and the second heterologous nucleic acid molecule encoding CXCL10. In some embodiments, the second population of encapsulated cells comprising an inducible kill switch and the second heterologous nucleic acid molecule encoding the second cytokine, comprises Caspase 9, and the second heterologous nucleic acid molecule encoding CXCL11. In some embodiments, the second population of encapsulated cells comprising an inducible kill switch and the second heterologous nucleic acid molecule encoding the second cytokine, comprises Caspase 9, and the second heterologous nucleic acid molecule encoding CXCL12. In some embodiments, the second population of encapsulated cells comprising an inducible kill switch and the second heterologous nucleic acid molecule encoding the second cytokine, comprises Caspase 9, and the second heterologous nucleic acid molecule encoding CXCL13. In some embodiments, the second population of encapsulated cells comprising an inducible kill switch and the second heterologous nucleic acid molecule encoding the second cytokine, comprises Caspase 9, and the second heterologous nucleic acid molecule encoding CXCL14. In some embodiments, the second population of encapsulated cells comprising an inducible kill switch and the second heterologous nucleic acid molecule encoding the second cytokine, comprises Caspase 9, and the second heterologous nucleic acid molecule encoding CXCL15. In some embodiments, the second population of encapsulated cells comprising an inducible kill switch and the second heterologous nucleic acid molecule encoding the second cytokine, comprises Caspase 9, and the second heterologous nucleic acid molecule encoding CXCL16. In some embodiments, the second population of encapsulated cells comprising an inducible kill switch and the second heterologous nucleic acid molecule encoding the second cytokine, comprises Caspase 9, and the second heterologous nucleic acid molecule encoding CXCL17. In some embodiments, the second population of encapsulated cells comprising an inducible kill switch and the second heterologous nucleic acid molecule encoding the second cytokine, comprises Caspase 9, and the second heterologous nucleic acid molecule encoding TRAIL. In some embodiments, the second population of encapsulated cells comprising an inducible kill switch and the second heterologous nucleic acid molecule encoding the second cytokine, comprises Caspase 9, and the second heterologous nucleic acid molecule encoding SCF. In some embodiments, the second population of encapsulated cells comprising an inducible kill switch and the second heterologous nucleic acid molecule encoding the second cytokine, comprises Caspase 9, and the second heterologous nucleic acid molecule encoding M-CSF. In some embodiments, the second population of encapsulated cells comprising an inducible kill switch and the second heterologous nucleic acid molecule encoding the second cytokine, comprises Caspase 9, and the second heterologous nucleic acid molecule encoding MSP (membrane scaffold protein). In some embodiments, the second population of encapsulated cells comprising an inducible kill switch and the second heterologous nucleic acid molecule encoding the second cytokine, comprises Caspase 9, and the second heterologous nucleic acid molecule encoding 4-1BBL. In some embodiments, the second population of encapsulated cells comprising an inducible kill switch and the second heterologous nucleic acid molecule encoding the second cytokine, comprises Caspase 9, and the second heterologous nucleic acid molecule encoding LIF (Leukemia Inhibitory Factor protein). In some embodiments, the second population of encapsulated cells comprising an inducible kill switch and the second heterologous nucleic acid molecule encoding the second cytokine, comprises Caspase 9, and the second heterologous nucleic acid molecule encoding OSM (Oncostatin M).

In some embodiments, the third population of encapsulated cells comprising an inducible kill switch and the third heterologous nucleic acid molecule encoding the third cytokine, comprises Caspase 9, and the third heterologous nucleic acid molecule encoding IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-β, IFN-γ, TNF-a, TNF-b, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, TRAIL, SCF, M-CSF, MSP (membrane scaffold protein), 4-1BBL, LIF (Leukemia Inhibitory Factor protein), or OSM (Oncostatin M). In some embodiments, the third population of encapsulated cells comprising an inducible kill switch and the third heterologous nucleic acid molecule encoding the third cytokine, comprises Caspase 9, and the third heterologous nucleic acid molecule encoding IL-1. In some embodiments, the third population of encapsulated cells comprising an inducible kill switch and the third heterologous nucleic acid molecule encoding the third cytokine, comprises Caspase 9, and the third heterologous nucleic acid molecule encoding IL-1a. In some embodiments, the third population of encapsulated cells comprising an inducible kill switch and the third heterologous nucleic acid molecule encoding the third cytokine, comprises Caspase 9, and the third heterologous nucleic acid molecule encoding IL-1b. In some embodiments, the third population of encapsulated cells comprising an inducible kill switch and the third heterologous nucleic acid molecule encoding the third cytokine, comprises Caspase 9, and the third heterologous nucleic acid molecule encoding IL-1RA. In some embodiments, the third population of encapsulated cells comprising an inducible kill switch and the third heterologous nucleic acid molecule encoding the third cytokine, comprises Caspase 9, and the third heterologous nucleic acid molecule encoding IL-2. In some embodiments, the third population of encapsulated cells comprising an inducible kill switch and the third heterologous nucleic acid molecule encoding the third cytokine, comprises Caspase 9, and the third heterologous nucleic acid molecule encoding IL-4. In some embodiments, the third population of encapsulated cells comprising an inducible kill switch and the third heterologous nucleic acid molecule encoding the third cytokine, comprises Caspase 9, and the third heterologous nucleic acid molecule encoding IL-5. In some embodiments, the third population of encapsulated cells comprising an inducible kill switch and the third heterologous nucleic acid molecule encoding the third cytokine, comprises Caspase 9, and the third heterologous nucleic acid molecule encoding IL-6. In some embodiments, the third population of encapsulated cells comprising an inducible kill switch and the third heterologous nucleic acid molecule encoding the third cytokine, comprises Caspase 9, and the third heterologous nucleic acid molecule encoding IL-7. In some embodiments, the third population of encapsulated cells comprising an inducible kill switch and the third heterologous nucleic acid molecule encoding the third cytokine, comprises Caspase 9, and the third heterologous nucleic acid molecule encoding IL-8. In some embodiments, the third population of encapsulated cells comprising an inducible kill switch and the third heterologous nucleic acid molecule encoding the third cytokine, comprises Caspase 9, and the third heterologous nucleic acid molecule encoding IL-9. In some embodiments, the third population of encapsulated cells comprising an inducible kill switch and the third heterologous nucleic acid molecule encoding the third cytokine, comprises Caspase 9, and the third heterologous nucleic acid molecule encoding IL-10. In some embodiments, the third population of encapsulated cells comprising an inducible kill switch and the third heterologous nucleic acid molecule encoding the third cytokine, comprises Caspase 9, and the third heterologous nucleic acid molecule encoding IL-11. In some embodiments, the third population of encapsulated cells comprising an inducible kill switch and the third heterologous nucleic acid molecule encoding the third cytokine, comprises Caspase 9, and the third heterologous nucleic acid molecule encoding IL-12. In some embodiments, the third population of encapsulated cells comprising an inducible kill switch and the third heterologous nucleic acid molecule encoding the third cytokine, comprises Caspase 9, and the third heterologous nucleic acid molecule encoding IL-12a. In some embodiments, the third population of encapsulated cells comprising an inducible kill switch and the third heterologous nucleic acid molecule encoding the third cytokine, comprises Caspase 9, and the third heterologous nucleic acid molecule encoding IL-12b. In some embodiments, the third population of encapsulated cells comprising an inducible kill switch and the third heterologous nucleic acid molecule encoding the third cytokine, comprises Caspase 9, and the third heterologous nucleic acid molecule encoding IL-13. In some embodiments, the third population of encapsulated cells comprising an inducible kill switch and the third heterologous nucleic acid molecule encoding the third cytokine, comprises Caspase 9, and the third heterologous nucleic acid molecule encoding IL-14. In some embodiments, the third population of encapsulated cells comprising an inducible kill switch and the third heterologous nucleic acid molecule encoding the third cytokine, comprises Caspase 9, and the third heterologous nucleic acid molecule encoding IL-15. In some embodiments, the third population of encapsulated cells comprising an inducible kill switch and the third heterologous nucleic acid molecule encoding the third cytokine, comprises Caspase 9, and the third heterologous nucleic acid molecule encoding IL-16. In some embodiments, the third population of encapsulated cells comprising an inducible kill switch and the third heterologous nucleic acid molecule encoding the third cytokine, comprises Caspase 9, and the third heterologous nucleic acid molecule encoding IL-17. In some embodiments, the third population of encapsulated cells comprising an inducible kill switch and the third heterologous nucleic acid molecule encoding the third cytokine, comprises Caspase 9, and the third heterologous nucleic acid molecule encoding IL-20. In some embodiments, the third population of encapsulated cells comprising an inducible kill switch and the third heterologous nucleic acid molecule encoding the third cytokine, comprises Caspase 9, and the third heterologous nucleic acid molecule encoding IFN-α. In some embodiments, the third population of encapsulated cells comprising an inducible kill switch and the third heterologous nucleic acid molecule encoding the third cytokine, comprises Caspase 9, and the third heterologous nucleic acid molecule encoding IFN-β. In some embodiments, the third population of encapsulated cells comprising an inducible kill switch and the third heterologous nucleic acid molecule encoding the third cytokine, comprises Caspase 9, and the third heterologous nucleic acid molecule encoding IFN-γ. In some embodiments, the third population of encapsulated cells comprising an inducible kill switch and the third heterologous nucleic acid molecule encoding the third cytokine, comprises Caspase 9, and the third heterologous nucleic acid molecule encoding TNF-a. In some embodiments, the third population of encapsulated cells comprising an inducible kill switch and the third heterologous nucleic acid molecule encoding the third cytokine, comprises Caspase 9, and the third heterologous nucleic acid molecule encoding TNF-b. In some embodiments, the third population of encapsulated cells comprising an inducible kill switch and the third heterologous nucleic acid molecule encoding the third cytokine, comprises Caspase 9, and the third heterologous nucleic acid molecule encoding TGF-b. In some embodiments, the third population of encapsulated cells comprising an inducible kill switch and the third heterologous nucleic acid molecule encoding the third cytokine, comprises Caspase 9, and the third heterologous nucleic acid molecule encoding CCL-1. In some embodiments, the third population of encapsulated cells comprising an inducible kill switch and the third heterologous nucleic acid molecule encoding the third cytokine, comprises Caspase 9, and the third heterologous nucleic acid molecule encoding CCL-2. In some embodiments, the third population of encapsulated cells comprising an inducible kill switch and the third heterologous nucleic acid molecule encoding the third cytokine, comprises Caspase 9, and the third heterologous nucleic acid molecule encoding CCL-3. In some embodiments, the third population of encapsulated cells comprising an inducible kill switch and the third heterologous nucleic acid molecule encoding the third cytokine, comprises Caspase 9, and the third heterologous nucleic acid molecule encoding CCL-4. In some embodiments, the third population of encapsulated cells comprising an inducible kill switch and the third heterologous nucleic acid molecule encoding the third cytokine, comprises Caspase 9, and the third heterologous nucleic acid molecule encoding CCL-5. In some embodiments, the third population of encapsulated cells comprising an inducible kill switch and the third heterologous nucleic acid molecule encoding the third cytokine, comprises Caspase 9, and the third heterologous nucleic acid molecule encoding CCL-6. In some embodiments, the third population of encapsulated cells comprising an inducible kill switch and the third heterologous nucleic acid molecule encoding the third cytokine, comprises Caspase 9, and the third heterologous nucleic acid molecule encoding CCL-7. In some embodiments, the third population of encapsulated cells comprising an inducible kill switch and the third heterologous nucleic acid molecule encoding the third cytokine, comprises Caspase 9, and the third heterologous nucleic acid molecule encoding CCL-8. In some embodiments, the third population of encapsulated cells comprising an inducible kill switch and the third heterologous nucleic acid molecule encoding the third cytokine, comprises Caspase 9, and the third heterologous nucleic acid molecule encoding CCL-9. In some embodiments, the third population of encapsulated cells comprising an inducible kill switch and the third heterologous nucleic acid molecule encoding the third cytokine, comprises Caspase 9, and the third heterologous nucleic acid molecule encoding CCL-10. In some embodiments, the third population of encapsulated cells comprising an inducible kill switch and the third heterologous nucleic acid molecule encoding the third cytokine, comprises Caspase 9, and the third heterologous nucleic acid molecule encoding CCL-11. In some embodiments, the third population of encapsulated cells comprising an inducible kill switch and the third heterologous nucleic acid molecule encoding the third cytokine, comprises Caspase 9, and the third heterologous nucleic acid molecule encoding CCL-12. In some embodiments, the third population of encapsulated cells comprising an inducible kill switch and the third heterologous nucleic acid molecule encoding the third cytokine, comprises Caspase 9, and the third heterologous nucleic acid molecule encoding CCL-13. In some embodiments, the third population of encapsulated cells comprising an inducible kill switch and the third heterologous nucleic acid molecule encoding the third cytokine, comprises Caspase 9, and the third heterologous nucleic acid molecule encoding CCL-14. In some embodiments, the third population of encapsulated cells comprising an inducible kill switch and the third heterologous nucleic acid molecule encoding the third cytokine, comprises Caspase 9, and the third heterologous nucleic acid molecule encoding CCL-15. In some embodiments, the third population of encapsulated cells comprising an inducible kill switch and the third heterologous nucleic acid molecule encoding the third cytokine, comprises Caspase 9, and the third heterologous nucleic acid molecule encoding CCL-16. In some embodiments, the third population of encapsulated cells comprising an inducible kill switch and the third heterologous nucleic acid molecule encoding the third cytokine, comprises Caspase 9, and the third heterologous nucleic acid molecule encoding CCL-17. In some embodiments, the third population of encapsulated cells comprising an inducible kill switch and the third heterologous nucleic acid molecule encoding the third cytokine, comprises Caspase 9, and the third heterologous nucleic acid molecule encoding CCL-18. In some embodiments, the third population of encapsulated cells comprising an inducible kill switch and the third heterologous nucleic acid molecule encoding the third cytokine, comprises Caspase 9, and the third heterologous nucleic acid molecule encoding CCL-19. In some embodiments, the third population of encapsulated cells comprising an inducible kill switch and the third heterologous nucleic acid molecule encoding the third cytokine, comprises Caspase 9, and the third heterologous nucleic acid molecule encoding CCL-20. In some embodiments, the third population of encapsulated cells comprising an inducible kill switch and the third heterologous nucleic acid molecule encoding the third cytokine, comprises Caspase 9, and the third heterologous nucleic acid molecule encoding CCL-21. In some embodiments, the third population of encapsulated cells comprising an inducible kill switch and the third heterologous nucleic acid molecule encoding the third cytokine, comprises Caspase 9, and the third heterologous nucleic acid molecule encoding CCL-22. In some embodiments, the third population of encapsulated cells comprising an inducible kill switch and the third heterologous nucleic acid molecule encoding the third cytokine, comprises Caspase 9, and the third heterologous nucleic acid molecule encoding CCL-23. In some embodiments, the third population of encapsulated cells comprising an inducible kill switch and the third heterologous nucleic acid molecule encoding the third cytokine, comprises Caspase 9, and the third heterologous nucleic acid molecule encoding CCL-24. In some embodiments, the third population of encapsulated cells comprising an inducible kill switch and the third heterologous nucleic acid molecule encoding the third cytokine, comprises Caspase 9, and the third heterologous nucleic acid molecule encoding CCL-25. In some embodiments, the third population of encapsulated cells comprising an inducible kill switch and the third heterologous nucleic acid molecule encoding the third cytokine, comprises Caspase 9, and the third heterologous nucleic acid molecule encoding CCL-26. In some embodiments, the third population of encapsulated cells comprising an inducible kill switch and the third heterologous nucleic acid molecule encoding the third cytokine, comprises Caspase 9, and the third heterologous nucleic acid molecule encoding CCL-27. In some embodiments, the third population of encapsulated cells comprising an inducible kill switch and the third heterologous nucleic acid molecule encoding the third cytokine, comprises Caspase 9, and the third heterologous nucleic acid molecule encoding CCL-28. In some embodiments, the third population of encapsulated cells comprising an inducible kill switch and the third heterologous nucleic acid molecule encoding the third cytokine, comprises Caspase 9, and the third heterologous nucleic acid molecule encoding CXCL1. In some embodiments, the third population of encapsulated cells comprising an inducible kill switch and the third heterologous nucleic acid molecule encoding the third cytokine, comprises Caspase 9, and the third heterologous nucleic acid molecule encoding CXCL2. In some embodiments, the third population of encapsulated cells comprising an inducible kill switch and the third heterologous nucleic acid molecule encoding the third cytokine, comprises Caspase 9, and the third heterologous nucleic acid molecule encoding CXCL3. In some embodiments, the third population of encapsulated cells comprising an inducible kill switch and the third heterologous nucleic acid molecule encoding the third cytokine, comprises Caspase 9, and the third heterologous nucleic acid molecule encoding CXCL5. In some embodiments, the third population of encapsulated cells comprising an inducible kill switch and the third heterologous nucleic acid molecule encoding the third cytokine, comprises Caspase 9, and the third heterologous nucleic acid molecule encoding CXCL6. In some embodiments, the third population of encapsulated cells comprising an inducible kill switch and the third heterologous nucleic acid molecule encoding the third cytokine, comprises Caspase 9, and the third heterologous nucleic acid molecule encoding CXCL7. In some embodiments, the third population of encapsulated cells comprising an inducible kill switch and the third heterologous nucleic acid molecule encoding the third cytokine, comprises Caspase 9, and the third heterologous nucleic acid molecule encoding CXCL8. In some embodiments, the third population of encapsulated cells comprising an inducible kill switch and the third heterologous nucleic acid molecule encoding the third cytokine, comprises Caspase 9, and the third heterologous nucleic acid molecule encoding CXCL9. In some embodiments, the third population of encapsulated cells comprising an inducible kill switch and the third heterologous nucleic acid molecule encoding the third cytokine, comprises Caspase 9, and the third heterologous nucleic acid molecule encoding CXCL10. In some embodiments, the third population of encapsulated cells comprising an inducible kill switch and the third heterologous nucleic acid molecule encoding the third cytokine, comprises Caspase 9, and the third heterologous nucleic acid molecule encoding CXCL11. In some embodiments, the third population of encapsulated cells comprising an inducible kill switch and the third heterologous nucleic acid molecule encoding the third cytokine, comprises Caspase 9, and the third heterologous nucleic acid molecule encoding CXCL12. In some embodiments, the third population of encapsulated cells comprising an inducible kill switch and the third heterologous nucleic acid molecule encoding the third cytokine, comprises Caspase 9, and the third heterologous nucleic acid molecule encoding CXCL13. In some embodiments, the third population of encapsulated cells comprising an inducible kill switch and the third heterologous nucleic acid molecule encoding the third cytokine, comprises Caspase 9, and the third heterologous nucleic acid molecule encoding CXCL14. In some embodiments, the third population of encapsulated cells comprising an inducible kill switch and the third heterologous nucleic acid molecule encoding the third cytokine, comprises Caspase 9, and the third heterologous nucleic acid molecule encoding CXCL15. In some embodiments, the third population of encapsulated cells comprising an inducible kill switch and the third heterologous nucleic acid molecule encoding the third cytokine, comprises Caspase 9, and the third heterologous nucleic acid molecule encoding CXCL16. In some embodiments, the third population of encapsulated cells comprising an inducible kill switch and the third heterologous nucleic acid molecule encoding the third cytokine, comprises Caspase 9, and the third heterologous nucleic acid molecule encoding CXCL17. In some embodiments, the third population of encapsulated cells comprising an inducible kill switch and the third heterologous nucleic acid molecule encoding the third cytokine, comprises Caspase 9, and the third heterologous nucleic acid molecule encoding TRAIL. In some embodiments, the third population of encapsulated cells comprising an inducible kill switch and the third heterologous nucleic acid molecule encoding the third cytokine, comprises Caspase 9, and the third heterologous nucleic acid molecule encoding SCF. In some embodiments, the third population of encapsulated cells comprising an inducible kill switch and the third heterologous nucleic acid molecule encoding the third cytokine, comprises Caspase 9, and the third heterologous nucleic acid molecule encoding M-CSF. In some embodiments, the third population of encapsulated cells comprising an inducible kill switch and the third heterologous nucleic acid molecule encoding the third cytokine, comprises Caspase 9, and the third heterologous nucleic acid molecule encoding MSP (membrane scaffold protein). In some embodiments, the third population of encapsulated cells comprising an inducible kill switch and the third heterologous nucleic acid molecule encoding the third cytokine, comprises Caspase 9, and the third heterologous nucleic acid molecule encoding 4-1BBL. In some embodiments, the third population of encapsulated cells comprising an inducible kill switch and the third heterologous nucleic acid molecule encoding the third cytokine, comprises Caspase 9, and the third heterologous nucleic acid molecule encoding LIF (Leukemia Inhibitory Factor protein). In some embodiments, the third population of encapsulated cells comprising an inducible kill switch and the third heterologous nucleic acid molecule encoding the third cytokine, comprises Caspase 9, and the third heterologous nucleic acid molecule encoding OSM (Oncostatin M).

In some embodiments, the fourth population of encapsulated cells comprising an inducible kill switch and the fourth heterologous nucleic acid molecule encoding an antibody that binds to a checkpoint inhibitor, comprises Caspase 9, and the fourth heterologous nucleic acid molecule encoding an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRB antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-PD-1 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-PD-L1 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-PD-L2 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-CTLA4 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-TIM3 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-LAG3 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-VISTA antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-BTLA antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-TIGIT antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-LAIR1 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-CD73 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-CD160 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-2B4 antibody. In some embodiments, the fourth heterologous nucleic acid molecule encoding population of encapsulated cells comprising an inducible kill switch and the fourth heterologous nucleic acid molecule encoding heterologous nucleic acid molecule encoding an antibody that binds to a checkpoint inhibitor, comprises Caspase 9, and the fourth heterologous nucleic acid molecule encoding an anti-TGFRβ antibody.

In some embodiments, the second population of encapsulated cells comprising an inducible kill switch and the second heterologous nucleic acid molecule encoding an antibody that binds to a checkpoint inhibitor, comprises Caspase 9, and the second heterologous nucleic acid molecule encoding an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-PD-1 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-PD-L1 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-PD-L2 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-CTLA4 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-TIM3 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-LAG3 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-VISTA antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-BTLA antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-TIGIT antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-LAIR1 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-CD73 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-CD160 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-2B4 antibody. In some embodiments, the second heterologous nucleic acid molecule encoding population of encapsulated cells comprising an inducible kill switch and the second heterologous nucleic acid molecule encoding heterologous nucleic acid molecule encoding an antibody that binds to a checkpoint inhibitor, comprises Caspase 9, and the second heterologous nucleic acid molecule encoding an anti-TGFRβ antibody.

### E. Compositions and Administration of Encapsulated Cells

The present disclosure pharmaceutical compositions comprising a population of encapsulated cells. In some embodiments, the population of encapsulated cells is provided in an effective amount in the composition. In some embodiments, the effective amount is a therapeutically effective amount. In some embodiments, the effective amount is a prophylactically effective amount.

Pharmaceutical compositions described herein can be prepared by any method known in the art of pharmacology. In general, such preparatory methods include the steps of bringing the composition into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the composition may be generally equal to the dosage of the antigenic and/or therapeutic agent which would be administered to a subject and/or a convenient fraction of such a dosage such as, for example, one-half or one-third of such a dosage.

In some embodiments, the pharmaceutical compositions are frozen or cryopreserved. In some embodiments, the pharmaceutical compositions are not frozen or not cryopreserved.

Relative amounts of the composition, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) of any component.

The composition and a pharmaceutical composition thereof may be administered or implanted orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally. In some embodiments, the composition is injected subcutaneously. In some embodiments, the composition is injected into the intraperitoneal space. In some embodiments, the composition is injected into the intraperitoneal space. In some embodiments, the composition is delivered to the subject using a device, e.g., a cannula or catheter.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For ophthalmic use, provided compounds, compositions, and devices may be formulated as micronized suspensions or in an ointment such as petrolatum.

For ophthalmic use, provided compounds, compositions, and devices may be formulated as micronized suspensions or in an ointment such as petrolatum.

In some embodiments, the release of an antigenic, therapeutic, or additional pharmaceutical agent is released in a sustained fashion. In order to prolong the effect of a particular agent, it is often desirable to slow the absorption of the agent from injection. This can be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The compositions provided herein are typically formulated in dosage unit form, e.g., single unit dosage form, for ease of administration and uniformity of dosage. It will be understood, however, that the total daily usage of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific therapeutic agent employed; and like factors well known in the medical arts.

The exact amount of encapsulated cells required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations). The composition may be administered at dosage levels sufficient to deliver from about 0.00001 mg/kg to about 100 mg/kg, from about 0.0001 mg/kg to about 100 mg/kg, from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 0.001 mg/kg to about 1 mg/kg, of subject body weight per day, one or more times a day, to obtain the desired therapeutic effect. It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

In some embodiments, the composition is administered to the subject at least once, at least two times, at least three times, at least four times, at least five times, at least six time, at least seven times, at least eight times, at least nine times, at least ten times, or more. In some embodiments, the composition is administered to the subject more than once, more than two times, more than three times, more than four times, more than five times, more than six time, more than seven times, more than eight times, more than nine times, more than ten times, or more. In some embodiments, the composition is administered to the subject at least once, at least two times, at least three times, at least four times, at least five times, at least six time, at least seven times, at least eight times, at least nine times, at least ten times, or more, as a subsequent administration. In some embodiments, the composition is administered to the subject more than once, more than two times, more than three times, more than four times, more than five times, more than six time, more than seven times, more than eight times, more than nine times, more than ten times, or more, as a subsequent administration. In some embodiments, the subsequent administration comprises administering the same composition. In some embodiments, the subsequent administration comprises administering a different composition.

The encapsulated cells can be prepared according to any known method. For example, in some embodiments, methods of preparing encapsulated cells producing a recombinant protein are provided. In some embodiments, the methods comprise feeding through a coaxial needle a first composition comprising a polymeric hydrogel and a second composition comprising cells to be encapsulated suspended in a polymeric hydrogel to drop into a crosslinking solution to form the encapsulated cells, wherein the crosslinking solution comprises a sugar alcohol, a buffer, a metal salt, and a surfactant.

In some embodiments, the method comprises washing the encapsulated cells produced according to the methods provided for herein in a buffer solution produced. In some embodiments, the washing step removes substantially all or all of the free barium or barium chloride. In some embodiments, the encapsulated cells prepared according to the methods provided herein are stored in a storage buffer, such as DMEM/F12 cell culture media. In some embodiments, the stored cells retain viability for at least 10, 20, or 30 days. In some embodiments, the storage buffer is substantially free of plasmalyte buffer. Also provided for herein, are a population of encapsulated cells prepared according to a method as provided for herein.

In some embodiments, a suspension of encapsulated cells is provided. In some embodiments, the suspension comprises a population of encapsulated cells as provided for herein. In some embodiments, the encapsulated cells are encapsulated by a polymeric hydrogel, and the suspension comprises a crosslinking solution that comprises a sugar alcohol, a buffer, a metal salt, and a surfactant. The cells can be any type of cell. In some embodiments, the cell is a mammalian cell. In some embodiments, the cell is an epithelial cell. In some embodiments, the cell is a RPE cell. In some embodiments, the cell is a ARPE-19 cell, ARPE-19-SEAP-2-neo cell, RPE-J cell, and hTERT RPE-1 cell. In some embodiments, the cell is an engineered RPE cell. In some embodiments, the engineered cell is derived from the ARPE-19 cell line. In some embodiments, the cell is as provided herein. In some embodiments, the surfactant is TWEEN 20 (polysorbate 20). In some embodiments, the buffer is HEPES buffer. In some embodiments, the sugar alcohol is mannitol. In some embodiments, the metal salt is barium chloride. In some embodiments, the suspension comprises a population of encapsulated cells as provided for herein, wherein the encapsulated cells are encapsulated by a polymeric hydrogel, and a storage buffer, such as DMEM/F12 cell culture media. In some embodiments, the suspension provided is substantially free of plasmalyte buffer.

In some embodiments, the composition comprises two or more populations of encapsulated cells. In some embodiments, the first population of encapsulated cells comprises an inducible kill switch and a first heterologous nucleic acid molecule encoding a first cytokine, and a second population of encapsulated cells comprises an inducible kill switch and a second heterologous nucleic acid molecule encoding a second cytokine. In some embodiments, the first cytokine is an IL-2 polypeptide, an IL-15 polypeptide, or any combination thereof. In some embodiments, the second cytokine is an IL-12 polypeptide. In some embodiments, the composition further comprises a third population of encapsulated cells comprising an inducible kill switch and a third heterologous nucleic acid molecule encoding a third cytokine. In some embodiments, the third cytokine is an IFN-α polypeptide. In some embodiments, the composition further comprises a fourth population of encapsulated cells comprising an inducible kill switch and a fourth heterologous nucleic acid molecule encoding an antibody that binds to a checkpoint inhibitor. In some embodiments, the antibody that binds to a checkpoint inhibitor is an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody. In some embodiments, the inducible kill switch is inducible Caspase 9. In some embodiments, the inducible Caspase 9 is induced by AP1903 (Rimiducid; [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl](2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl)butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate). In some embodiments, the one or more populations of encapsulated cells are separately encapsulated. In some embodiments, the cells are encapsulated with a polymeric hydrogel selected from chitosan, cellulose, hyaluronic acid, alginate (e.g., a sodium alginate solution), or any combination thereof.

In some embodiments, a pharmaceutical composition is provided comprising a composition as disclosed herein.

### F. Methods of Treatment

Provided are methods of treating a subject with a disease with the composition as disclosed herein. In some embodiments, the disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In some embodiments, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In some embodiments, the cancer is ovarian cancer, uterine cancer, pancreatic cancer, or mesothelioma.

In some embodiments, the method of treating a subject with a disease comprises implanting in the intraperitoneal space of the subject a pharmaceutical composition comprising a population of encapsulated cells (e.g., a capsule) as provided for herein, whereby the pharmaceutical composition stimulates the activation of immune cells in the intraperitoneal space and the activated immune cells migrate to a region of the subject that is distal to the intraperitoneal space to treat the cancer systemically in the subject. In some embodiments, the pharmaceutical composition activates immune cells in the IP space. In some embodiments, the activated immune cells migrate out of (away from) the intraperitoneal space to treat the cancer in the subject at a site that is not in the IP space. In some embodiments, the activated immune cells migrate out of (away from) the intraperitoneal space to treat the cancer in the subject at a site that is distal from the IP space. In some embodiments, the site is another organ or tissue, such as pancreas, breast, brain, lungs, bone, or as otherwise provided for herein.

In some embodiments, a method of providing systemic treatment to a subject with cancer, is provided. In some embodiment, the method comprises implanting in the intraperitoneal space of the subject a composition comprising a plurality of a the population of encapsulated cells (e.g., a capsule) as provided for herein, whereby the composition stimulates the activation of immune cells in the intraperitoneal space and the activated immune cells migrate to a region of the subject that is distal to the intraperitoneal space to treat the cancer systemically in the subject. In some embodiment, the method comprises implanting in the intraperitoneal space of the subject a pharmaceutical composition comprising a plurality of a the population of encapsulated cells (e.g., a capsule) as provided for herein, whereby the pharmaceutical composition stimulates the activation of immune cells in the intraperitoneal space and the activated immune cells migrate to a region of the subject that is distal to the intraperitoneal space to treat the cancer systemically in the subject.

In some embodiments, methods of providing systemic treatment to a subject with cancer are provided. In some embodiments, the methods comprise implanting in the intraperitoneal space of the subject a pharmaceutical composition comprising a plurality of a the population of encapsulated cells (e.g., a capsules) as provided herein. In some embodiments, the pharmaceutical composition activates immune cells in the IP space. In some embodiments, the activated immune cells migrate out of (away from) the intraperitoneal space to treat the cancer in the subject at a site that is not in the IP space. In some embodiments, the activated immune cells migrate out of (away from) the intraperitoneal space to treat the cancer in the subject at a site that is distal from the IP space. In some embodiments, the site is another organ or tissue, such as pancreas, breast, brain, lungs, bone, or as otherwise provided for herein.

In some embodiments, the subject is administered (e.g., implanted) about 0.01 ug/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.05 μg/kg/day, about 0.1 μg/kg/day, about 0.5 ug/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about 16 μg/kg/day, about 17 μg/kg/day, about 18 μg/kg/day, about 19 μg/kg/day, or about 20 μg/kg/day, of the encapsulated cells as provided herein.

As described herein, the encapsulated cells producing the recombinant native human IL-12 can be used to create memory immunity against a tumor. Thus, in some embodiments, the methods provided herein can be used to prevent or reduce the probability of a tumor recurring either at the initial site of the tumor or a site that is distal to the origin of the tumor. In some embodiments, the tumor is a pancreatic tumor. In some embodiments, the methods of treating a tumor by generating (inducing) memory immunity comprise implanting a pharmaceutical composition comprising a population of encapsulated cells as provided for herein.

Exemplary cancers that can be treated by the methods provided for herein include, but are not limited to, carcinoma, lymphoma, blastoma, sarcoma, and leukemia or lymphoid malignancies. In some embodiments, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Other examples of cancers include, without limitation, carcinoma, lymphoma, blastoma, sarcoma, and leukemia or lymphoid malignancies. In some embodiments, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Other examples of cancers include, without limitation: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In some embodiments, the composition (encapsulated cells) described herein may be used in a method to modulate (e.g., upregulate) the immune response in a subject. For example, upon administration to a subject, the composition (or an antigenic and/or therapeutic agent disposed within) may modulate (e.g., upregulate) the level of a component of the immune system in a subject (e.g., increasing the level or decreasing the level of an immune system component). Exemplary immune system components that may be modulated by a composition or related method described herein include stem cells (hematopoietic stem cells), NK cells, T cells (e.g., an adaptive T cell (e.g., a helper T cell, a cytotoxic T cell, memory T cell, or regulatory T cell) or an innate-like T cell (e.g., natural killer T cell, mucosal-associated invariant T cell, or gamma delta T cell), B cells, an antibody or fragment thereof, or other another component. In some embodiments, the modulation comprises increasing or decreasing the activation of a T cell or other immune system component (e.g., by about 5%, 10%, 15%, 20%, 25%, 30%, 35%, 40%, 45%, 50%, 55%, 60%, 65%, 70%, 75%, 80%, 85%, 90%, 95%, 99%, or more compared with a control). In some embodiments, the encapsulated cells can be used to activate CD4 positive and/or CD8 positive immune cells.

The compositions described herein may be used to modulate the immune response in a subject for a specific period of time. For example, administration of the compositions (or an antigenic and/or therapeutic agent disposed within) may activate the immune response (e.g., by increase in the level of an immune system component) in a subject for at least 1 hour, 2 hours, 3 hours, 4 hours, 6 hours, 8 hours, 10 hours, 12 hours, 16 hours, 20 hours, 1 day, 1.5 days, 2 days, 3 days, 4 days, 5 days, 6 days, 1 week, 1.5 weeks, 2 weeks, 3 weeks, 4 weeks, 5 weeks, 6 weeks, 2 months, 2.5 months, 3 months, 4 months, 5 months, 6 months, or longer. In some embodiments, administration of the composition activates the immune response (e.g., by increase in the level of an immune system component) in a subject between 1 hour and 1 month, 1 hour and 3 weeks, 1 hour and 2 weeks, 1 hour and 1 week, 6 hours and 1 week, or 6 hours and 3 days. In some embodiments, implantation of the composition (e.g., a composition described herein) results in upregulation of T cells in a subject, e.g., as measured by a blood test, for at least 1 day.

### G. Enumerated Embodiments

1. A composition comprising a population of encapsulated cells comprising a heterologous nucleic acid molecule encoding a protein of interest and an inducible kill switch.
2. The composition of embodiment 1, wherein the inducible kill switch is inducible Caspase 9.
3. The composition of embodiment 2, wherein the inducible Caspase 9 is induced by AP1903 (Rimiducid; [(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).
4. The composition of embodiments 1 or 2, wherein the protein of interest is selected from IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-β, IFN-γ, TNF-α, TNF-β, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, G-CSF, GM-CSF, IL-23, CD154, LT-B, CD70, CD153, CD178, TRAIL, SCF, M-CSF, MSP (membrane scaffold protein), 4-1BBL, LIF (Leukemia Inhibitory Factor protein), OSM (Oncostatin M), or any combination thereof.
5. The composition of embodiments 1 or 2, wherein the protein of interest is IFN-α.
6. The composition of embodiments 1 or 2, wherein the protein of interest is an antibody that binds to a checkpoint inhibitor.
7. The composition of embodiment 6, wherein the protein of interest is selected from an anti-PD-1 antibody, anti-PD-L1 antibody, anti-PD-L2 antibody, anti-CTLA-4 antibody, anti-TIM3 antibody, anti-LAG-3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody.
8. The composition of embodiment 7, wherein the protein of interest is selected from an anti-PD-1 antibody, anti-PD-L1 antibody, anti-CTLA-4 antibody, or anti-LAG-3 antibody.
9. The composition of embodiment 7, wherein the anti-PD-1 antibody is selected from pembrolizumab, nivolumab, cemiplimab, atezolizumab, dostralimab, durvalumab, avelumab, or any combination thereof.
10. The composition of any one of embodiments 1-8, wherein the cells are retinal pigment epithelial cells.
11. The composition of any one of embodiments 1-10, wherein the cells are ARPE-19 cells, ARPE-19-SEAP-2-neo cells, RPE-J cells, hTERT RPE-1 cells, or any combination thereof.
12. A composition comprising a population of encapsulated cells comprising a heterologous nucleic acid molecule encoding an IL-2 and an inducible kill switch.
13. The compositions of embodiment 12, wherein the heterologous nucleic acid molecule encoding the IL-2 comprises a sequence of SEQ ID NO: 1 or SEQ ID NO: 24, or codon optimized nucleic acid sequence thereof.
14. The composition of embodiment 13, wherein the codon optimized nucleic acid sequence encoding the IL-2 comprises a sequence having at least 90%, 91%, 92%, 93%, 94%, 95%, 96%, 97%, 98%, or 99% sequence identity to SEQ ID NO: 1 or SEQ ID NO: 24.
15. The composition of any one of embodiments 12-14, wherein the cell produces recombinant IL-2 protein.
16. The composition of embodiment 15, wherein the recombinant IL-2 protein has the amino acid sequence of SEQ ID NO: 6.
17. The composition of embodiment 12, wherein the inducible kill switch is inducible Caspase 9.
18. The composition of embodiment 17, wherein the inducible Caspase 9 is induced by AP1903 (Rimiducid; [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).
19. The composition of any one of embodiments 12-18, wherein the cells are retinal pigment epithelial cells.
20. The composition of any one of embodiments 12-19, wherein the cells are ARPE-19 cells, ARPE-19-SEAP-2-neo cells, RPE-J cells, hTERT RPE-1 cells, or any combination thereof.
21. A composition comprising a population of encapsulated cells comprising a plurality of heterologous nucleic acid molecules encoding an IL-12 and an inducible kill switch.
22. The composition of embodiment 21, wherein the IL-12 is a heterodimeric complex comprising a IL-12a polypeptide and a IL-12b polypeptide.
23. The composition of any one of embodiments 21-22, wherein the nucleic acid encoding the IL-12a polypeptide comprises a sequence of SEQ ID NO: 7 or SEQ ID NO: 25, and the nucleic acid encoding the IL-12b polypeptide comprises a sequence of SEQ ID NO: 8 or SEQ ID NO: 26, respectively.
24. The composition of any one of embodiments 21-23, wherein the expressed IL-12a polypeptide and the expressed IL-12b polypeptide form a heterodimeric complex.
25. The compositions of any one of embodiments 21-24, wherein the heterologous nucleic acid molecule encoding an IL-12 comprises a sequence of SEQ ID NO: 7 and SEQ ID NO: 8, or SEQ ID NO: 25 and SEQ ID NO: 26, or codon optimized nucleic acid sequence thereof.
26. The composition of embodiment 25, wherein the codon optimized nucleic acid sequence encoding an IL-12 comprises a sequence having at least 90%, 91%, 92%, 93%, 94%, 95%, 96%, 97%, 98%, or 99% sequence identity to SEQ ID NO: 7 and SEQ ID NO: 8, or SEQ ID NO: 25 and SEQ ID NO: 26.
27. The composition of embodiment 21, wherein the inducible kill switch is inducible Caspase 9.
28. The composition of embodiment 24, wherein the inducible Caspase 9 is induced by AP1903 (Rimiducid; [(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).
29. The composition of any one of embodiments 21-28, wherein the cells are retinal pigment epithelial cells.
30. The composition of any one of embodiments 21-29, wherein the cells are ARPE-19 cells, ARPE-19-SEAP-2-neo cells, RPE-J cells, hTERT RPE-1 cells, or any combination thereof.
31. A composition comprising two or more populations of encapsulated cells:
wherein a first population of encapsulated cells comprises an inducible kill switch and a first heterologous nucleic acid molecule encoding a first cytokine; and
a second population of encapsulated cells comprises an inducible kill switch and a second heterologous nucleic acid molecule encoding a second cytokine, or an antibody that binds to a checkpoint inhibitor.
32. The composition of embodiment 31, further comprising a third population of encapsulated cells comprising an inducible kill switch and a third heterologous nucleic acid molecule encoding a third cytokine.
33. The composition of embodiment 32, further comprising a fourth population of encapsulated cells comprising an inducible kill switch and a fourth heterologous nucleic acid molecule encoding an antibody that binds to a checkpoint inhibitor.
34. The composition of embodiment 31, wherein the first cytokine is selected from an IL-2 polypeptide, an IL-15 polypeptide, or any combination thereof.
35. The composition of embodiment 31, wherein the second cytokine is an IL-12 polypeptide.
36. The composition of embodiment 32, wherein the third cytokine is an IFN-α polypeptide.
37. The composition of any one of embodiments 31-36, wherein the antibody that binds to a checkpoint inhibitor is selected from an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody.
38. The composition of any of embodiments 31-37, wherein the inducible kill switch is inducible Caspase 9.
39. The composition of embodiment 38, wherein the inducible Caspase 9 is induced by AP1903 (Rimiducid; [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[[2-[[[2-[3-[(1R)-3-(3,4-dimethoxyphenyl)-1-[(2S)-1-[(2S)- 2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).
40. The composition of any one of embodiments 31-39, wherein the cells are retinal pigment epithelial cells.
41. The composition of any one of embodiments 31-40, wherein the cells are ARPE-19 cells, ARPE-19-SEAP-2-neo cells, RPE-J cells, hTERT RPE-1 cells, or any combination thereof.
42. A composition comprising two or more populations of encapsulated cells:
wherein a first population of encapsulated cells comprises an inducible kill switch and a first plurality of heterologous nucleic acid molecules encoding an IL-12; and
a second population of encapsulated cells comprises an inducible kill switch and a second heterologous nucleic acid molecule encoding a second cytokine, or an antibody that binds to a checkpoint inhibitor.
43. The composition of embodiment 42, wherein the second cytokine is selected from IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-β, IFN-γ, TNF-α, TNF-β, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, G-CSF, GM-CSF, IL-23, CD154, LT-B, CD70, CD153, CD178, TRAIL, SCF, M-CSF, MSP (membrane scaffold protein), 4-1BBL, LIF (Leukemia Inhibitory Factor protein), OSM (Oncostatin M), or any combination thereof.
44. The composition of embodiment 43, wherein the second cytokine is an IFN-α polypeptide.
45. The composition of embodiment 42, further comprising a third population of encapsulated cells comprising an inducible kill switch and a third heterologous nucleic acid molecule encoding an antibody that binds to a checkpoint inhibitor.
46. The composition of embodiment 45, wherein the antibody that binds to a checkpoint inhibitor is selected from an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody.
47. The composition of any of embodiments 42-46, wherein the inducible kill switch is inducible Caspase 9.
48. The composition of embodiment 47, wherein the inducible Caspase 9 is induced by AP1903 (Rimiducid; [(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).
49. The composition of any one of embodiments 42-48, wherein the cells are retinal pigment epithelial cells.
50. The composition of any one of embodiments 42-49, wherein the cells are ARPE-19 cells, ARPE-19-SEAP-2-neo cells, RPE-J cells, hTERT RPE-1 cells, or any combination thereof.
51. The composition of any one of embodiments 31-50, wherein each of the two or more populations of encapsulated cells are separately encapsulated.
52. The composition of any of the preceding embodiments, wherein the cells are encapsulated with a polymeric hydrogel selected from chitosan, cellulose, hyaluronic acid, alginate, or any combination thereof.
53. The composition of embodiment 52, wherein the alginate is a sodium alginate solution.
54. The composition of any one of embodiments 52-53, wherein the alginate is SLG20.
55. A pharmaceutical composition comprising a composition of any one of the preceding embodiments.
56. A method of treating a subject with a disease, comprising administering to the subject the composition of any of the preceding embodiments.
57. The method of embodiment 56, wherein the disease is cancer.
58. The method of embodiment 57, wherein the cancer is ovarian cancer, uterine cancer, pancreatic cancer, or mesothelioma.
59. A method of treating a subject with a disease by generating memory immunity, the method comprising implanting in the subject a pharmaceutical composition comprising the population of encapsulated cells of any one of embodiments 1-54, or the pharmaceutical composition of embodiment 55.
60. The method of embodiment 59, wherein the disease is cancer.
61. The method of embodiment 60, wherein the cancer is ovarian cancer, uterine cancer, pancreatic cancer, or mesothelioma.
62. A method of providing systemic treatment to a subject with cancer, the method comprising implanting in the intraperitoneal space of the subject the composition of any one of embodiments 1-54, or the pharmaceutical composition of embodiment 55, whereby the composition or the pharmaceutical composition stimulates the activation of immune cells in the intraperitoneal space and the immune cells migrate to a region of the subject that is distal to the intraperitoneal space to treat the cancer systematically in the subject.
63. The method of embodiment 62, wherein the subject has fewer side effects as compared to a subject that is administered the composition of any one of embodiments 1-54, or the pharmaceutical composition of embodiment 55, systemically, such as intravenously.
64. The method of any one of embodiments 62-63, wherein the activated immune cells are CD8 positive effector T cells.
65. The method of embodiment 64, wherein the effector T cells are selectively activated and expanded at least 1, 2, 3, 4, or 5 times as compared to Tregs in the intraperitoneal space.
66. The method of any one of embodiments 64-65, wherein the effector T cells are selectively activated and expanded at least 1, 2, 3, 4, or 5 times as compared to Tregs systematically.
67. The method of any one of embodiments 56-66, wherein the composition of any one of embodiments 1-54, or the pharmaceutical composition of embodiment 55 is administered to the subject at least once.
68. The method of any one of embodiments 56-67, wherein the composition of any one of embodiments 1-54, or the pharmaceutical composition of embodiment 55 is administered to the subject more than once.
69. The method of any one of embodiments 56-68, wherein the composition of any one of embodiments 1-54, or the pharmaceutical composition of embodiment 55 is administered to the subject at least once, or more than once, is the same.
70. The method of any one of embodiments 56-68, wherein the composition of any one of embodiments 1-54, or the pharmaceutical composition of embodiment 55 is administered to the subject at least once, or more than once, is different.

### H. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

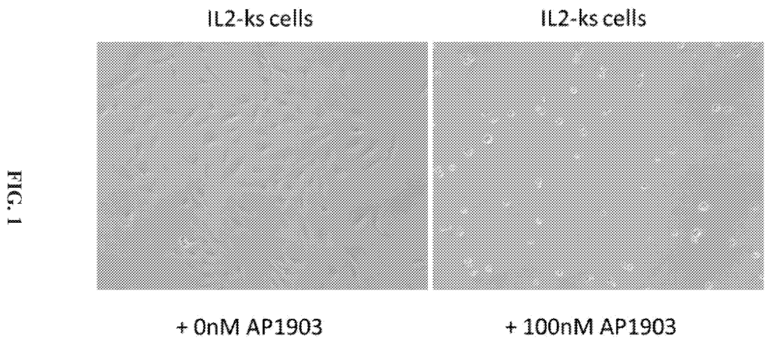

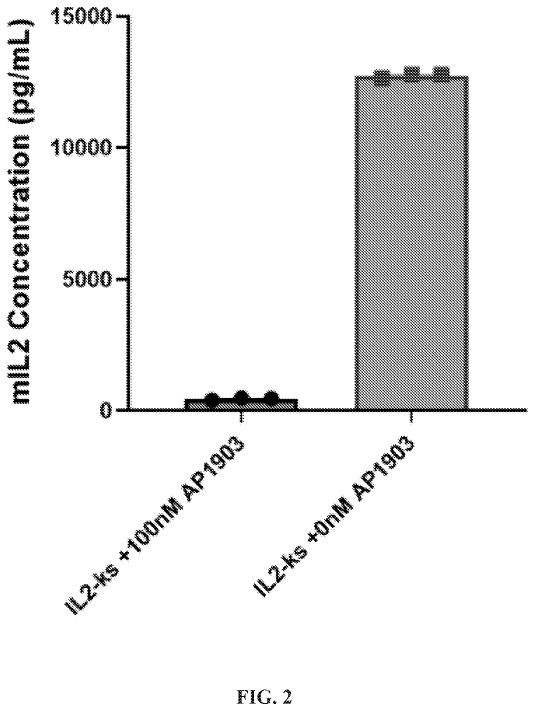

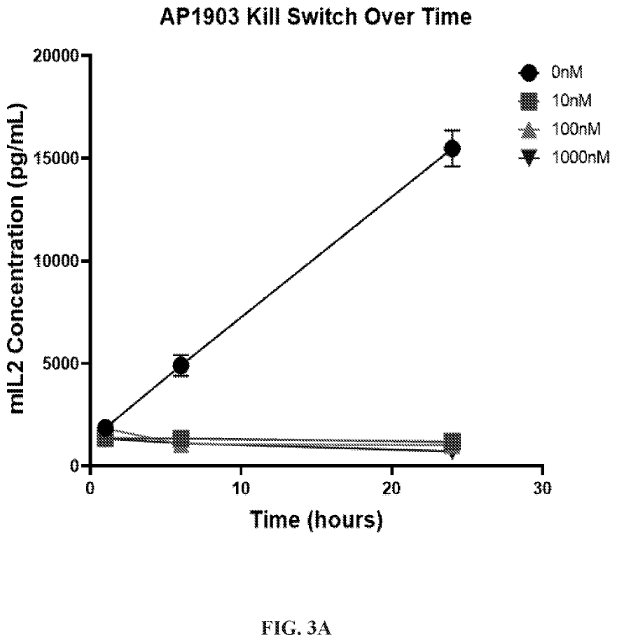

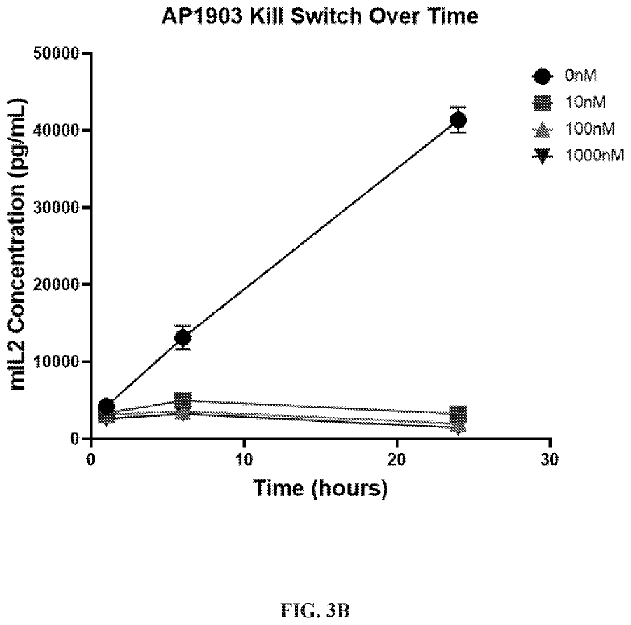

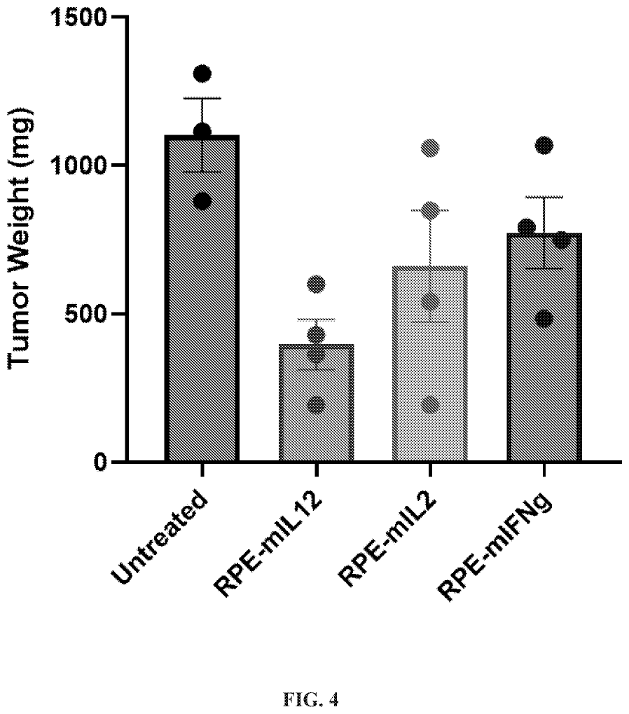

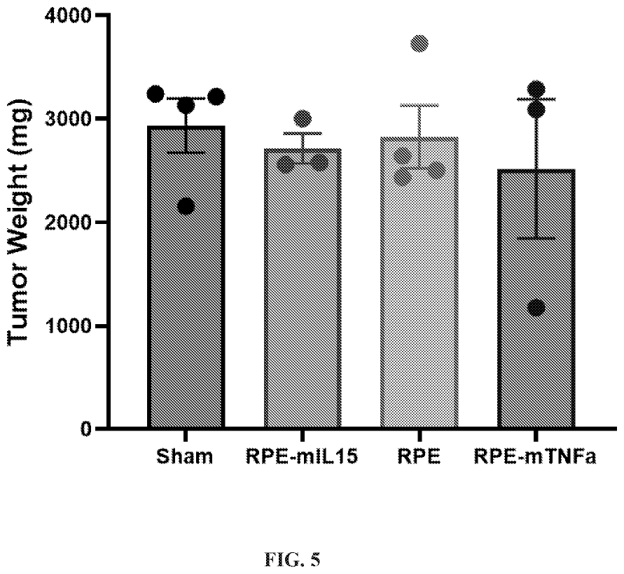

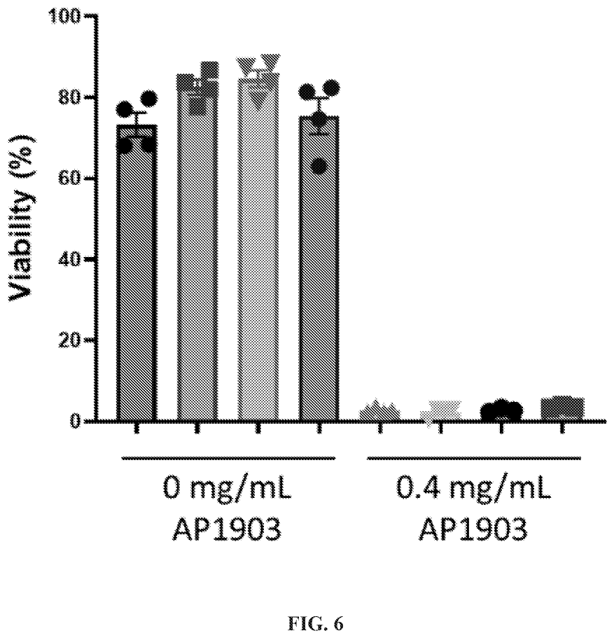

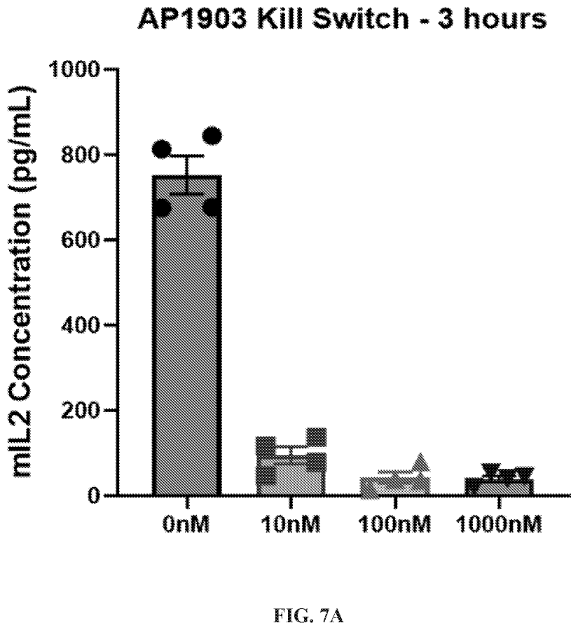

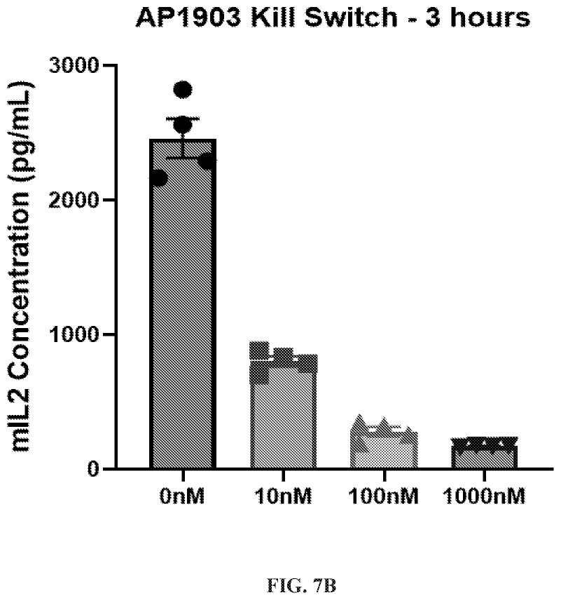

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain alike or similar result without departing from the spirit and scope of the disclosure.
Example 1: Cells expressing IL-2 and an inducible kill switch (IL2-ks) cells were treated with 100 nM AP1903. Bright field images of IL2-ks cells in vitro were taken 18 hours following administration of 100 nM AP1903. Images were acquired at 20× magnification. FIG. 1 shows images taken at 18 hours following treatment and reveals that administration of 100 nM AP1903 induced cell death, which demonstrates that the kill switch can be activated.
Example 2: Cells expressing IL-2 and an inducible kill switch (IL2-ks) cells were treated with 100 nM AP1903. The mIL-2 concentration from cell supernatant was assayed 18 hours after AP1903 administration (n=3 wells), using ELISA (R&D Systems). The results, shown in FIG. 2, demonstrate that AP1903 induces a significant reduction in cytokine production, which demonstrates that the kill switch can be activated.
Example 3: Cells expressing IL-2 and an inducible kill switch (IL2-ks) cells were treated with 10 nM, 100 nM, or 1000 nM AP1903. The IL-2 concentration from cell supernatant was assayed 3 hours after AP1903 administration (n=3 wells), using ELISA (R&D Systems). The results, shown in FIG. 7A (10.5×10⁶ cells/mL) and FIG. 7B (42×10\hat{ }6 cells/mL), demonstrate that AP1903 induces a significant reduction in cytokine production, which demonstrates that the kill switch can be activated.
Example 4: Encapsulated cells expressing IL-2 and an inducible kill switch (IL2-ks) cells were treated with 10 nM, 100 nM, or 1000 nM AP1903. The mIL-2 concentration was assessed over time from capsule supernatant (10.5M cells/mL capsule density) after AP1903 administration (n=4 capsules) using an ELISA (R&D Systems). As shown in FIG. 3A (10.5×10⁶ cells/mL) and FIG. 3B (42×10⁶ cells/mL), the cells responded rapidly to 10 nM, 100 nM, or 1000 nM AP1903 administration and the IL-2 production was reduced indicating that the kill-switch induced by AP1903 was active.
Example 5: Mice bearing intraperitoneal melanoma tumors were administered RPE-mIL12, RPE-mIL2, RPE-mIFNg capsules, or were untreated (n=3-4 animals/groups). As shown in FIG. 4, administration of RPE-mIL12 capsules caused a >50% reduction in tumor burden, compared to untreated control animals, after 1 week of treatment (n=3-4 animals/group). Administration of RPE-mIL2 and RPE-mIFNg capsules caused a modest reduction of tumor burden, as compared to untreated control animals, suggesting that combination treatment may enhance the response to these treatments for melanoma.
Example 6: Mice bearing intraperitoneal melanoma tumors were administered RPE-mIL15, RPE, RPE-mTNFa, or sham surgeries (n=3-4 animals/group). As shown in FIG. 5, administration of RPE-mIL15, RPE, or RPE-mTNFa capsules did not reduce tumor burden, compared to sham animals, after 1 week of treatment (n=3-4 animals/group).
Example 7: Viability of explanted IL2-ks capsules was assessed following overnight incubation with or without 0.4 mg/mL AP1903 administered to the intraperitoneal space of healthy C57BL/6 mice (n=4-5 mice per group, mixed gender). As shown in FIG. 6, capsules explanted from animals not given AP1903 maintained high cell viability (>70% live) following explanation and capsule lysis while the capsules explanted from animals that received 0.4 mg/mL AP1903 displayed low viability (<10% live) following explanation and capsule lysis. Thus, the data shows that encapsulated IL2-ks cells respond to 0.4 mg/mL AP1903 in less than 16 hours in healthy mice.

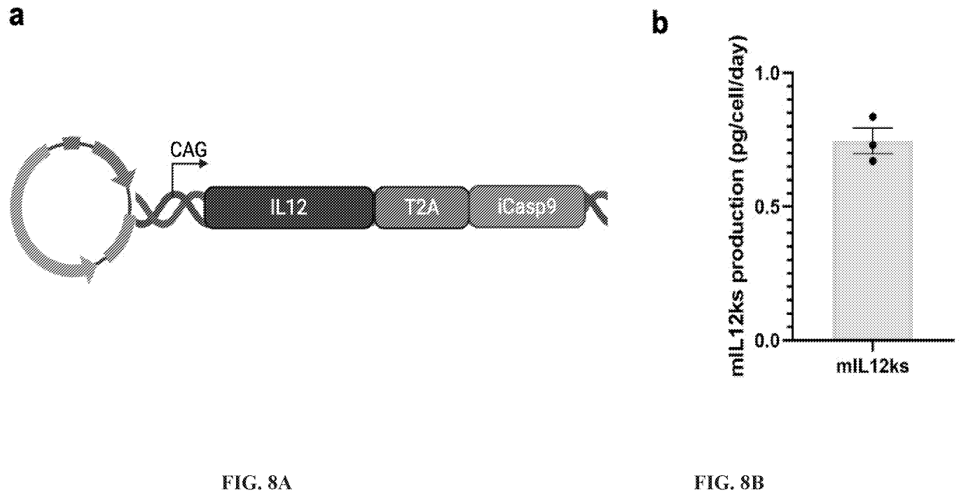

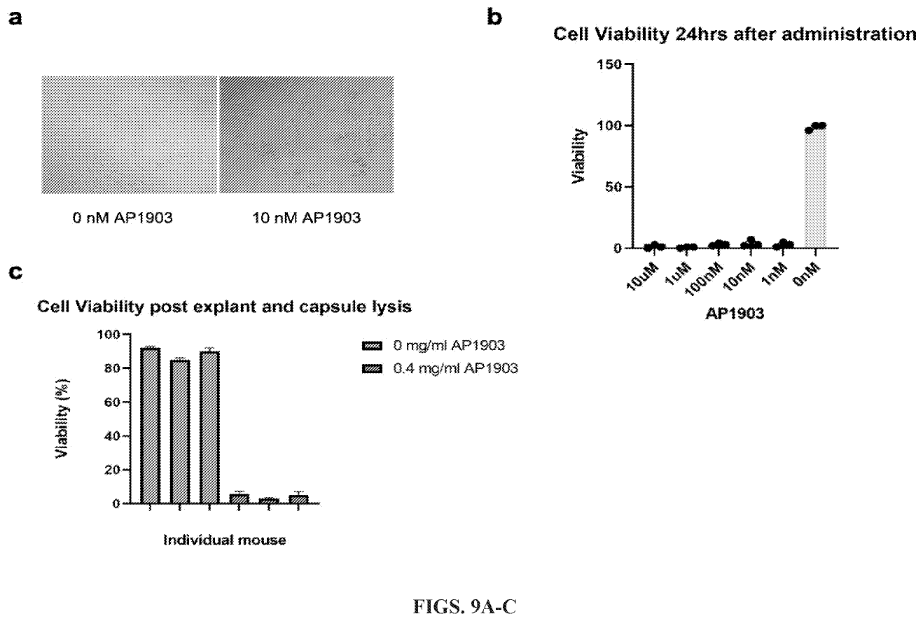

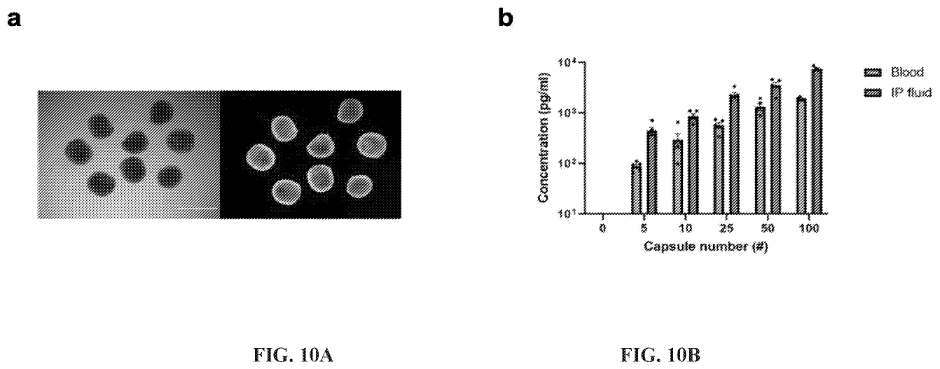

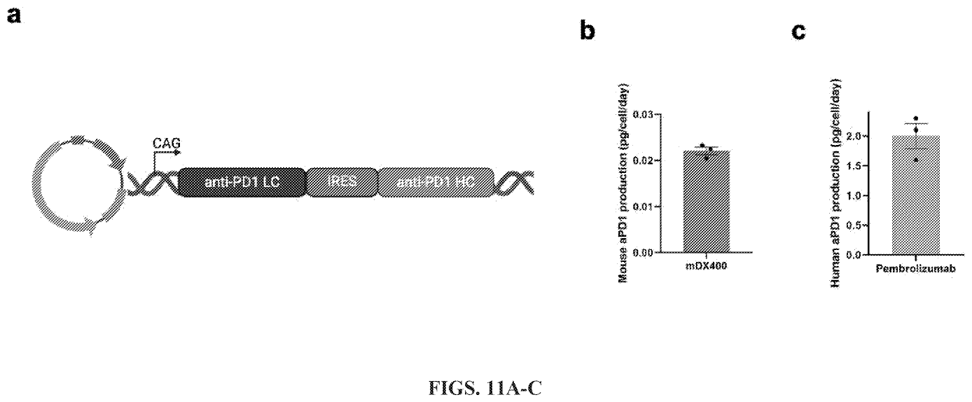

The embodiments and examples provided herein demonstrate that an inducible kill switch can function to limit the product of the protein being expressed by the encapsulated cells. Certain proteins, such as cytokines, can produce certain toxicities if production is not controlled. It was surprising and unexpected that that a kill switch in an encapsulated cell can be regulated and activated according to the embodiments provided herein.
Example 8: FIG. 8A is a graph of RPE-mIL12-ks plasmid design using a piggybac transposase system to create stable cell lines. FIG. 8B is a graph of mIL-12 production (pg/cell/day) of the cell. 10,000 engineered cells were plated, allowed to adhere and incubated with fresh media for 24 hours. Production was measured by running a protein specific ELISA on the supernatant. FIG. 9A is series of two annotated photographs of two populations of cytokine-producing, kill switch-inducible cells, the first population (left) not having been exposed to small molecule kill switch inducer AP1903, and the second population (right) 24 hours after exposure to 10 nM AP1903. FIG. 9B is the administration of different concentrations of CID (AP1903) in mIL 12 ks cells in a 24-well plate for 24 hours. Addition of kill switch leads to 99% cell death 24 hour after treatment. FIG. 9C is the validation of AP1903 treatment in inducing encapsulated cell death in mice. The inventors implant 50 mIL12 ks capsules in each mouse and administrate half of the mice with AP1903 right after implantation surgeries. Euthanize all the mice after 24 hours, retrieve capsules and lysis capsules for measuring cell viability. All three mice treated with AP1903 decreased cell viability as compared to the control mice. FIG. 10B is a graph of in vivo mouse IL-12 concentration as a function of capsule dose. The inventors determined that the local IP concentration of mIL-12 increased with the number of capsules delivered and that the mIL-12 levels in the IP fluid were 10× higher than in the blood.

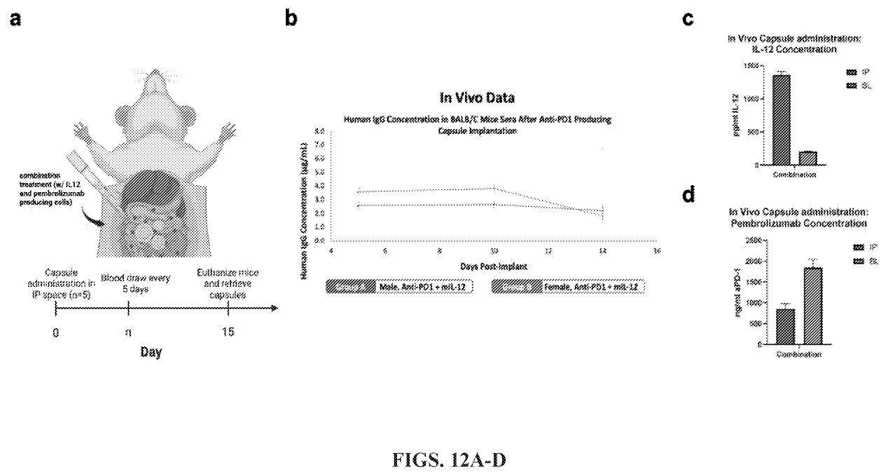

FIG. 11A is a graph of RPE-aPD1 plasmid design for producing either human anti-PD1 antibody (Pembrolizumab) or mouse anti-PD1 antibody (mDX400). FIGS. 11B-C are graphs of IgG production (pg/cell/day) level of the cells. 10,000 engineered cells were plated, allowed to adhere and incubated with fresh media for 24 hours. Production was measured by running a protein specific ELISA on the supernatant from each cell type. FIGS. 12A-D show a study on cytokine and aPD-1 antibodies combination treatment in healthy mice model over 14 days. The inventors implanted capsules with mouse IL-12 and pembrolizumab producing cells in the IP cavity of balb/c mice. They collected blood samples every 5 days after administration and euthanize the mice in day 15. The graph in the middle shows the pembrolizumab concentration in serum after capsules implantation. The results demonstrate the ability of this platform to constantly deliver antibodies in vivo, both in systemic circulation and locally. The graph on the right shows the mouse IL-12 concentration and anti-PD-1 antibody concentration in either blood samples or IP fluid samples in day 15 post-implantation. The inventors determined that capsules implanted within the IP cavity created high IL-12 concentrations within the IP cavity while maintaining low concentrations in the blood, which means that there's no excessive accumulation of cytokine in the blood. For anti-PD1 antibody level in local, it is lower in IP space than in blood but still comparable. This study confirmed the delivery of both cytokine and anti-PD1 antibodies over 14 days.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A composition comprising a population of encapsulated cells comprising a heterologous nucleic acid molecule encoding a protein of interest and an inducible kill switch.

2. The composition of claim 1, wherein the inducible kill switch is inducible Caspase 9.

3. The composition of claim 2, wherein the inducible Caspase 9 is induced by AP1903 (Rimiducid; [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).

4. The composition of claim 1 or 2, wherein the protein of interest is selected from IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-β, IFN-γ, TNF-α, TNF-β, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, G-CSF, GM-CSF, IL-23, CD154, LT-β, CD70, CD153, CD178, TRAIL, SCF, M-CSF, MSP (membrane scaffold protein), 4-1BBL, LIF (Leukemia Inhibitory Factor protein), OSM (Oncostatin M), or any combination thereof.

5. The composition of claim 1 or 2, wherein the protein of interest is IFN-α.

6. The composition of claim 1 or 2, wherein the protein of interest is an antibody that binds to a checkpoint inhibitor.

7. The composition of claim 6, wherein the protein of interest is selected from an anti-PD-1 antibody, anti-PD-L1 antibody, anti-PD-L2 antibody, anti-CTLA-4 antibody, anti-TIM3 antibody, anti-LAG-3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody.

8. The composition of claim 7, wherein the protein of interest is selected from an anti-PD-1 antibody, anti-PD-L1 antibody, anti-CTLA-4 antibody, or anti-LAG-3 antibody.

9. The composition of claim 7, wherein the anti-PD-1 antibody is selected from pembrolizumab, nivolumab, cemiplimab, atezolizumab, dostralimab, durvalumab, avelumab, or any combination thereof.

10. The composition of any one of claims 1-8, wherein the cells are retinal pigment epithelial cells.

11. The composition of any one of claims 1-10, wherein the cells are ARPE-19 cells, ARPE-19-SEAP-2-neo cells, RPE-J cells, hTERT RPE-1 cells, or any combination thereof.

12. A composition comprising a population of encapsulated cells comprising a heterologous nucleic acid molecule encoding an IL-2 and an inducible kill switch.

13. The compositions of claim 12, wherein the heterologous nucleic acid molecule encoding the IL-2 comprises a sequence of SEQ ID NO: 1 or SEQ ID NO: 24, or codon optimized nucleic acid sequence thereof.

14. The composition of claim 13, wherein the codon optimized nucleic acid sequence encoding the IL-2 comprises a sequence having at least 90%, 91%, 92%, 93%, 94%, 95%, 96%, 97%, 98%, or 99% sequence identity to SEQ ID NO: 1 or SEQ ID NO: 24.

15. The composition of any one of claims 12-14, wherein the cell produces recombinant IL-2 protein.

16. The composition of claim 15, wherein the recombinant IL-2 protein has the amino acid sequence of SEQ ID NO: 6.

17. The composition of claim 12, wherein the inducible kill switch is inducible Caspase 9.

18. The composition of claim 17, wherein the inducible Caspase 9 is induced by AP1903 (Rimiducid; [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).

19. The composition of any one of claims 12-18, wherein the cells are retinal pigment epithelial cells.

20. The composition of any one of claims 12-19, wherein the cells are ARPE-19 cells, ARPE-19-SEAP-2-neo cells, RPE-J cells, hTERT RPE-1 cells, or any combination thereof.

21. A composition comprising a population of encapsulated cells comprising a plurality of heterologous nucleic acid molecules encoding an IL-12 and an inducible kill switch.

22. The composition of claim 21, wherein the IL-12 is a heterodimeric complex comprising a IL-12a polypeptide and a IL-12b polypeptide.

23. The composition of any one of claims 21-22, wherein the nucleic acid encoding the IL-12a polypeptide comprises a sequence of SEQ ID NO: 7 or SEQ ID NO: 25, and the nucleic acid encoding the IL-12b polypeptide comprises a sequence of SEQ ID NO: 8 or SEQ ID NO: 26, respectively.

24. The composition of any one of claims 21-23, wherein the expressed IL-12a polypeptide and the expressed IL-12b polypeptide form a heterodimeric complex.

25. The compositions of any one of claims 21-24, wherein the heterologous nucleic acid molecule encoding an IL-12 comprises a sequence of SEQ ID NO: 7 and SEQ ID NO: 8, or SEQ ID NO: 25 and SEQ ID NO: 26, or codon optimized nucleic acid sequence thereof.

26. The composition of claim 25, wherein the codon optimized nucleic acid sequence encoding an IL-12 comprises a sequence having at least 90%, 91%, 92%, 93%, 94%, 95%, 96%, 97%, 98%, or 99% sequence identity to SEQ ID NO: 7 and SEQ ID NO: 8, or SEQ ID NO: 25 and SEQ ID NO: 26.

27. The composition of claim 21, wherein the inducible kill switch is inducible Caspase 9.

28. The composition of claim 24, wherein the inducible Caspase 9 is induced by AP1903 (Rimiducid; [(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).

29. The composition of any one of claims 21-28, wherein the cells are retinal pigment epithelial cells.

30. The composition of any one of claims 21-29, wherein the cells are ARPE-19 cells, ARPE-19-SEAP-2-neo cells, RPE-J cells, hTERT RPE-1 cells, or any combination thereof.

31. A composition comprising two or more populations of encapsulated cells:
wherein a first population of encapsulated cells comprises an inducible kill switch and a first heterologous nucleic acid molecule encoding a first cytokine; and
a second population of encapsulated cells comprises an inducible kill switch and a second heterologous nucleic acid molecule encoding a second cytokine, or an antibody that binds to a checkpoint inhibitor.

32. The composition of claim 31, further comprising a third population of encapsulated cells comprising an inducible kill switch and a third heterologous nucleic acid molecule encoding a third cytokine.

33. The composition of claim 32, further comprising a fourth population of encapsulated cells comprising an inducible kill switch and a fourth heterologous nucleic acid molecule encoding an antibody that binds to a checkpoint inhibitor.

34. The composition of claim 31, wherein the first cytokine is selected from an IL-2 polypeptide, an IL-15 polypeptide, or any combination thereof.

35. The composition of claim 31, wherein the second cytokine is an IL-12 polypeptide.

36. The composition of claim 32, wherein the third cytokine is an IFN-α polypeptide.

37. The composition of any one of claims 31-36, wherein the antibody that binds to a checkpoint inhibitor is selected from an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody.

38. The composition of any of claims 31-37, wherein the inducible kill switch is inducible Caspase 9.

39. The composition of claim 38, wherein the inducible Caspase 9 is induced by AP1903 (Rimiducid; [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[2-[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).

40. The composition of any one of claims 31-39, wherein the cells are retinal pigment epithelial cells.

41. The composition of any one of claims 31-40, wherein the cells are ARPE-19 cells, ARPE-19-SEAP-2-neo cells, RPE-J cells, hTERT RPE-1 cells, or any combination thereof.

42. A composition comprising two or more populations of encapsulated cells:
wherein a first population of encapsulated cells comprises an inducible kill switch and a first plurality of heterologous nucleic acid molecules encoding an IL-12; and
a second population of encapsulated cells comprises an inducible kill switch and a second heterologous nucleic acid molecule encoding a second cytokine, or an antibody that binds to a checkpoint inhibitor.

43. The composition of claim 42, wherein the second cytokine is selected from IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-β, IFN-γ, TNF-α, TNF-β, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, G-CSF, GM-CSF, IL-23, CD154, LT-β, CD70, CD153, CD178, TRAIL, SCF, M-CSF, MSP (membrane scaffold protein), 4-1BBL, LIF (Leukemia Inhibitory Factor protein), OSM (Oncostatin M), or any combination thereof.

44. The composition of claim 43, wherein the second cytokine is an IFN-α polypeptide.

45. The composition of claim 42, further comprising a third population of encapsulated cells comprising an inducible kill switch and a third heterologous nucleic acid molecule encoding an antibody that binds to a checkpoint inhibitor.

46. The composition of claim 45, wherein the antibody that binds to a checkpoint inhibitor is selected from an anti-PD-1 antibody, anti-PD-L1 antibody, anti-PD-L2 antibody, anti-CTLA4 antibody, anti-TIM3 antibody, anti-LAG3 antibody, anti-VISTA antibody, anti-BTLA antibody, anti-TIGIT antibody, anti-LAIR1 antibody, anti-CD73 antibody, anti-CD160 antibody, anti-2B4 antibody, or anti-TGFRβ antibody.

47. The composition of any of claims 42-46, wherein the inducible kill switch is inducible Caspase 9.

48. The composition of claim 47, wherein the inducible Caspase 9 is induced by AP1903 (Rimiducid; [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl) butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl) butanoyl]piperidine-2-carboxylate) or AP20187 ([(1R)-3-(3,4-dimethoxyphenyl)-1-[3-[2-[[2-[[[2-[3-[(1R)-3-(3,4-dimethoxyphenyl)-1-[(2S)-1-[(2S)- 2-(3,4,5-trimethoxyphenyl) butanoyl]piperidine-2-carbonyl]oxypropyl]phenoxy]acetyl]amino]methyl]-3-(dimethylamino) propyl]amino]-2-oxoethoxy]phenyl]propyl] (2S)-1-[(2S)-2-(3,4,5-trimethoxyphenyl) butanoyl]piperidine-2-carboxylate).

49. The composition of any one of claims 42-48, wherein the cells are retinal pigment epithelial cells.

50. The composition of any one of claims 42-49, wherein the cells are ARPE-19 cells, ARPE-19-SEAP-2-neo cells, RPE-J cells, hTERT RPE-1 cells, or any combination thereof.

51. The composition of any one of claims 31-50, wherein each of the two or more populations of encapsulated cells are separately encapsulated.

52. The composition of any of the preceding claims, wherein the cells are encapsulated with a polymeric hydrogel selected from chitosan, cellulose, hyaluronic acid, alginate, or any combination thereof.

53. The composition of claim 52, wherein the alginate is a sodium alginate solution.

54. The composition of any one of claims 52-53, wherein the alginate is SLG20.

55. A pharmaceutical composition comprising a composition of any one of the preceding claims.

56. A method of treating a subject with a disease, comprising administering to the subject the composition of any of the preceding claims.

57. The method of claim 56, wherein the disease is cancer.

58. The method of claim 57, wherein the cancer is ovarian cancer, uterine cancer, pancreatic cancer, or mesothelioma.

59. A method of treating a subject with a disease by generating memory immunity, the method comprising implanting in the subject a pharmaceutical composition comprising the population of encapsulated cells of any one of claims 1-54, or the pharmaceutical composition of claim 55.

60. The method of claim 59, wherein the disease is cancer.

61. The method of claim 60, wherein the cancer is ovarian cancer, uterine cancer, pancreatic cancer, or mesothelioma.

62. A method of providing systemic treatment to a subject with cancer, the method comprising implanting in the intraperitoneal space of the subject the composition of any one of claims 1-54, or the pharmaceutical composition of claim 55, whereby the composition or the pharmaceutical composition stimulates the activation of immune cells in the intraperitoneal space and the immune cells migrate to a region of the subject that is distal to the intraperitoneal space to treat the cancer systematically in the subject.

63. The method of claim 62, wherein the subject has fewer side effects as compared to a subject that is administered the composition of any one of claims 1-54, or the pharmaceutical composition of claim 55, systemically, such as intravenously.

64. The method of any one of claims 62-63, wherein the activated immune cells are CD8 positive effector T cells.

65. The method of claim 64, wherein the effector T cells are selectively activated and expanded at least 1, 2, 3, 4, or 5 times as compared to Tregs in the intraperitoneal space.

66. The method of any one of claims 64-65, wherein the effector T cells are selectively activated and expanded at least 1, 2, 3, 4, or 5 times as compared to Tregs systematically.

67. The method of any one of claims 56-66, wherein the composition of any one of claims 1-54, or the pharmaceutical composition of claim 55 is administered to the subject at least once.

68. The method of any one of claims 56-67, wherein the composition of any one of claims 1-54, or the pharmaceutical composition of claim 55 is administered to the subject more than once.

69. The method of any one of claims 56-68, wherein the composition of any one of claims 1-54, or the pharmaceutical composition of claim 55 is administered to the subject at least once, or more than once, is the same.

70. The method of any one of claims 56-68, wherein the composition of any one of claims 1-54, or the pharmaceutical composition of claim 55 is administered to the subject at least once, or more than once, is different.


SEQUENCE LISTING (26 sequences)

SEQ ID NO: 1 - DNA, 462 bp, Homo sapiens
  source 1..462: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 2 - DNA, 462 bp, Homo sapiens
  source 1..462: mol_type = other DNA
atgtaccggatgcagctgctgtcctgcatcgcactgtccctcgccctggtgacaaattctgcccccacctcctccagcacaaaaaagacccagttgcagctggagcacctgctgctggatctgcagatgatcctgaatggcatcaataactacaaaaaccctaaactgaccagaatgctgacctttaaattttacatgcctaaaaaggcaaccgagctgaagcacctgcagtgcctggaagaggaactgaagcccctggaggaggtgctgaacctggcccagagcaagaactttcacctgcggccccgcgacctgatcagcaacatcaacgtgatcgtgctggagctgaagggcagtgaaaccacattcatgtgcgagtacgccgacgagaccgccacaatcgtggagttcctgaacagatggatcacattctgtcagtccatcattagcacactgacctaa

SEQ ID NO: 3 - DNA, 462 bp, Homo sapiens
  source 1..462: mol_type = other DNA
atgtaccgcatgcagctgctgagctgcatcgccctgagcctggccctggtgaccaacagcgcccccaccagcagcagcaccaagaagacccagctgcagctggagcacctgctgctggacctgcagatgatcctgaacggcatcaacaactacaagaaccccaagctgacccgcatgctgaccttcaagttctacatgcccaagaaggccaccgagctgaagcacctgcagtgcctggaggaggagctgaagcccctggaggaggtgctgaacctggcccagagcaagaacttccacctgcgcccccgcgacctgatcagcaacatcaacgtgatcgtgctggagctgaagggcagcgagaccaccttcatgtgcgagtacgccgacgagaccgccaccatcgtggagttcctgaaccgctggatcaccttctgccagagcatcatcagcaccctgacctaa

SEQ ID NO: 4 - DNA, 459 bp, Homo sapiens
  source 1..459: mol_type = other DNA
atgtataggatgcagctgctctcttgtatcgcgttgtctctggctttggtgactaactcagctcccacgtccagcagtaccaaaaagacccagctgcagctggaacatcttctgttggatctgcaaatgatactgaatgggatcaacaactataaaaacccaaaactgactagaatgctgactttcaagttctacatgcctaaaaaggcaacagaattgaagcaccttcagtgcctggaggaggagcttaagcccctggaggaggtgctgaatctggcccaaagtaagaattttcatctgcgacccagggatctgatcagtaatatcaatgtgatcgtcctggagctgaagggcagtgagaccacgtttatgtgtgaatacgcagacgaaaccgccactatcgttgaattcttgaacaggtggatcaccttttgtcagagtatcatcagcaccctcact

SEQ ID NO: 5 - DNA, 462 bp, Homo sapiens
  source 1..462: mol_type = other DNA
atgtacagaatgcagctgctgagctgcatcgccctgagcctggccctggtgaccaacagcgcccccacaagcagcagcaccaagaagacacagctgcagctggagcacctgctgctggacctgcagatgatcctgaacggcatcaacaactacaagaaccccaagctgacaagaatgctgaccttcaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccaccttcatgtgcgagtacgccgacgagaccgccaccatcgtggagttcctgaacagatggatcaccttctgtcagagcatcatcagcaccctgacctga

SEQ ID NO: 6 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - DNA, 1444 bp, Homo sapiens
  source 1..1444: mol_type = other DNA
atttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtga

SEQ ID NO: 8 - DNA, 2364 bp, Homo sapiens
  source 1..2364: mol_type = other DNA
agaagaaacaacatctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatcata

SEQ ID NO: 9 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 11 - protein, 335 aa, Mus sp.
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 12-13 - skipped (2 entries)

SEQ ID NO: 14 - DNA, 489 bp, Homo sapiens
  source 1..489: mol_type = other DNA
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctga

SEQ ID NO: 15 - DNA, 561 bp, Homo sapiens
  source 1..561: mol_type = other DNA
atggctaggctctgtgctttcctcatgatcctggtaatgatgagctactactggtcagcctgttctctaggatgtgacctgcctcacacttataacctcgggaacaagagggccttgacagtcctggaagaaatgagaagactcccccctctttcctgcctgaaggacaggaaggattttggattccccttggagaaggtggataaccaacagatccagaaggctcaagccatccttgtgctaagagatcttacccagcagattttgaacctcttcacatcaaaagacttgtctgctacttggaatgcaactctactagactcattctgcaatgacctccatcagcagctcaatgacctcaaagcctgtgtgatgcaggaacctcctctgacccaggaagactccctgctggctgtgaggacatacttccacaggatcactgtgtacctgagaaagaagaaacacagcctctgtgcctgggaggtgatcagagcagaagtctggagagccctctcttcctcaaccaacttgctggcaagactgagtgaggagaaggagtga

SEQ ID NO: 16 - DNA, 468 bp, Homo sapiens
  source 1..468: mol_type = other DNA
atgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctga

SEQ ID NO: 17 - DNA, 708 bp, Homo sapiens
  source 1..708: mol_type = other DNA
atgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtga

SEQ ID NO: 18 - DNA, 12464 bp, Homo sapiens
  source 1..12464: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtacaggatgcaactcctgtcttgcattgcactaagtcttgcacttgtcacgaattcgcaggtgcagctggtggaaagcggcggcggcgtggtgcagccgggccgcagcctgcgcctgagctgcgcggcgagcggctttacctttagcagctataccatgcattgggtgcgccaggcgccgggcaaaggcctggaatgggtgacctttattagctatgatggcaacaacaaatattatgcggatagcgtgaaaggccgctttaccattagccgcgataacagcaaaaacaccctgtatctgcagatgaacagcctgcgcgcggaagataccgcgatttattattgcgcgcgcaccggctggctgggcccgtttgattattggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaacgcgtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtacaggatgcaactcctgtcttgcattgcactaagtcttgcacttgtcacgaattcggaaattgtgctgacccagagcccgggcaccctgagcctgagcccgggcgaacgcgcgaccctgagctgccgcgcgagccagagcgtgggcagcagctatctggcgtggtatcagcagaaaccgggccaggcgccgcgcctgctgatttatggcgcgtttagccgcgcgaccggcattccggatcgctttagcggcagcggcagcggcaccgattttaccctgaccattagccgcctggaaccggaagattttgcggtgtattattgccagcagtatggcagcagcccgtggacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 19 - DNA, 12464 bp, Homo sapiens
  source 1..12464: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtacaggatgcaactcctgtcttgcattgcactaagtcttgcacttgtcacgaattcgcaggtgcagctggtggaaagcggcggcggcgtggtgcagccgggccgcagcctgcgcctgagctgcgcggcgagcggctttacctttagcagctataccatgcattgggtgcgccaggcgccgggcaaaggcctggaatgggtgacctttattagctatgatggcaacaacaaatattatgcggatagcgtgaaaggccgctttaccattagccgcgataacagcaaaaacaccctgtatctgcagatgaacagcctgcgcgcggaagataccgcgatttattattgcgcgcgcaccggctggctgggcccgtttgattattggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaacgcgtggaaccgaaaagctgcgataaaacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtacaggatgcaactcctgtcttgcattgcactaagtcttgcacttgtcacgaattcggaaattgtgctgacccagagcccgggcaccctgagcctgagcccgggcgaacgcgcgaccctgagctgccgcgcgagccagagcgtgggcagcagctatctggcgtggtatcagcagaaaccgggccaggcgccgcgcctgctgatttatggcgcgtttagccgcgcgaccggcattccggatcgctttagcggcagcggcagcggcaccgattttaccctgaccattagccgcctggaaccggaagattttgcggtgtattattgccagcagtatggcagcagcccgtggacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 20 - DNA, 12470 bp, Homo sapiens
  source 1..12470: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtacaggatgcaactcctgtcttgcattgcactaagtcttgcacttgtcacgaattcgcaggtgcagctggtgcagagcggcgtggaagtgaaaaaaccgggcgcgagcgtgaaagtgagctgcaaagcgagcggctatacctttaccaactattatatgtattgggtgcgccaggcgccgggccagggcctggaatggatgggcggcattaacccgagcaacggcggcaccaactttaacgaaaaatttaaaaaccgcgtgaccctgaccaccgatagcagcaccaccaccgcgtatatggaactgaaaagcctgcagtttgatgataccgcggtgtattattgcgcgcgccgcgattatcgctttgatatgggctttgattattggggccagggcaccaccgtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtacaggatgcaactcctgtcttgcattgcactaagtcttgcacttgtcacgaattcggaaattgtgctgacccagagcccggcgaccctgagcctgagcccgggcgaacgcgcgaccctgagctgccgcgcgagcaaaggcgtgagcaccagcggctatagctatctgcattggtatcagcagaaaccgggccaggcgccgcgcctgctgatttatctggcgagctatctggaaagcggcgtgccggcgcgctttagcggcagcggcagcggcaccgattttaccctgaccattagcagcctggaaccggaagattttgcggtgtattattgccagcatagccgcgatctgccgctgacctttggcggcggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 21 - DNA, 12470 bp, Homo sapiens
  source 1..12470: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatgtacaggatgcaactcctgtcttgcattgcactaagtcttgcacttgtcacgaattcgcaggtgcagctggtgcagagcggcgtggaagtgaaaaaaccgggcgcgagcgtgaaagtgagctgcaaagcgagcggctatacctttaccaactattatatgtattgggtgcgccaggcgccgggccagggcctggaatggatgggcggcattaacccgagcaacggcggcaccaactttaacgaaaaatttaaaaaccgcgtgaccctgaccaccgatagcagcaccaccaccgcgtatatggaactgaaaagcctgcagtttgatgataccgcggtgtattattgcgcgcgccgcgattatcgctttgatatgggctttgattattggggccagggcaccaccgtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtacaggatgcaactcctgtcttgcattgcactaagtcttgcacttgtcacgaattcggaaattgtgctgacccagagcccggcgaccctgagcctgagcccgggcgaacgcgcgaccctgagctgccgcgcgagcaaaggcgtgagcaccagcggctatagctatctgcattggtatcagcagaaaccgggccaggcgccgcgcctgctgatttatctggcgagctatctggaaagcggcgtgccggcgcgctttagcggcagcggcagcggcaccgattttaccctgaccattagcagcctggaaccggaagattttgcggtgtattattgccagcatagccgcgatctgccgctgacctttggcggcggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 22 - DNA, 2340 bp, Homo sapiens
  source 1..2340: mol_type = other DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaa

SEQ ID NO: 23 - DNA, 3570 bp, Homo sapiens
  source 1..3570: mol_type = other DNA
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggaagcggagagggcaggggaagtcttctaacatgcggggacgtggaggaaaatcccggccccatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctagacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaa

SEQ ID NO: 24 - DNA, 510 bp, Homo sapiens
  source 1..510: mol_type = other DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 25 - DNA, 711 bp, Homo sapiens
  source 1..711: mol_type = other DNA
atggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 26 - DNA, 1008 bp, Homo sapiens
  source 1..1008: mol_type = other DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag